(19) United States
(12) Patent Application Publication
(10) Pub. No.: US 2025/0243512 A1
(43) Pub. Date: Jul. 31, 2025

(54) SERINE RECOMBINASE SYSTEMS FOR SITE-SPECIFIC GENE EDITING

(71) Applicant: Sangamo Therapeutics, Inc., Brisbane, CA (US)

(72) Inventors: Sabastian Arangundy, Albany, CA (US); Friedrich A. Fauser, Fremont, CA (US); Jeffrey C. Miller, San Rafael, CA (US); Nicholas Scarlott, Walnut Creek, CA (US)

(73) Assignee: Sangamo Therapeutics, Inc., Brisbane, CA (US)

(21) Appl. No.: 18/730,738

(22) PCT Filed: Jan. 27, 2023
(86) PCT No.: PCT/US2023/061500

§ 371 Date: Jul. 19, 2024

Related U.S. Application Data
(60) Provisional application No. 63/304,565, filed on Jan. 28, 2022.

(51) Int. Cl. C12N 15/90; C12N 9/22; C12N 15/11

(52) U.S. Cl. CPC C12N 15/902; C12N 9/22; C12N 15/111; C07K 2319/81; C12Y 301/22

(57) ABSTRACT

The present disclosure provides engineered large serine recombinases and systems containing the recombinases for gene editing.

### CROSS REFERENCE TO RELATED APPLICATIONS

The present application claims priority from U.S. Provisional Application 63/304,565, filed Jan. 28, 2022, the content of which is incorporated herein by reference in its entirety.

### SEQUENCE LISTING

The instant application contains a Sequence Listing that has been submitted electronically in XML format. The Sequence Listing is hereby incorporated by reference in its entirety. The XML copy, created on Jan. 26, 2023, is named 025297.WO043.xml and is 361,227 bytes in size.

### BACKGROUND OF THE INVENTION

Large serine recombinases (LSRs) are a family of enzymes encoded by temperate phages. These enzymes can precisely cut and recombine DNA in a site-specific manner, moving DNA elements into and out of bacterial chromosomes between short DNA attachment sites in the phage (attP) and in the host bacteria (attB) (Duyne and Rutherford, Crit Rev Biochem Mol Biol. (2013) 48 (5): 476-91; Smith, Microbiol Spectr. (2015) 3 (4): doi10.1128/microbiolspec). The highly directional and controlled process of DNA recombination mediated by LSRs render them a potential tool in genetic engineering and gene therapy where site-specific DNA integration, excision, inversion, or cassette exchange in a genome is desired.

Bxb1 recombinase, also known as Bxb1 integrase, is an LSR encoded by phage Bxb1 that facilitates integration of the phage DNA into the Mycobacterium smegmatis genome (Russell et al., Biotechniques (2018) 40 (4): doi.org/10.2144/000112150). The recombinase contains an N-terminal catalytic domain similar to that of smaller resolvases/invertases and a larger C-terminal domain responsible for coordination of activities unique to LSRs. This C-terminal domain is further divided into a recombinase domain (RD) and a Zinc ribbon domain (ZD) that contains a coiled-coil (CC) motif (Rutherford et al., Nucleic Acids Res. (2013) 41 (17): 8341-56). During recombination events, the ZD and RD domains of a Bxb1 recombinase dimer bind to an attachment site (attB or attP) on a double-stranded DNA segment, while the ZD and RD domains of another Bxb1 recombinase dimer bind to the other attachment site (attP or attB) on another double-stranded DNA segment. The subsequent complexing of the two dimers leads to the formation of a Bxb1 recombinase tetramer and brings together the attP and attB sites on the two DNA segments. Serine residues at the tetramer's active sites form covalent bonds at those center dinucleotide base pairs and cleave the double-stranded DNA. This cleavage makes the center dinucleotides into 3′ overhangs. The resulting protein/DNA complex is rotated and the previously separate DNA molecules or segments are ligated if the center dinucleotides at the attB and attP sequences are identical. This process produces attachment R (attR) and attachment (attL) sites that are no longer substrates for the recombinase without the presence of additional co-factors.

Despite the precision of gene editing afforded by large serine recombinases, their utility as a genetic engineering and therapeutic tool has been limited by the rarity of their binding sites in the human genome, including in therapeutically relevant genes. Thus, there remains a need for new recombinases that can recognize a variety of DNA attachment sites and realize the therapeutic potential of this category of enzymes.

### SUMMARY OF THE INVENTION

The present disclosure provides large serine recombinase variants that have altered DNA target specificity compared to their wildtype counterparts. In one aspect, the disclosure provides a non-naturally occurring variant of Bxb1 recombinase with altered DNA target specificity relative to wildtype Bxb1 recombinase (e.g., SEQ ID NO: 1), comprising one or more amino acid mutations (e.g., substitutions, deletions, or insertions) within a ZD or a RD.

In some embodiments, the one or more amino acid mutations occur at one or more of positions 147, 148, 149, 154, 155, 156, 158, 197, 198, 230, 231, 232, 233, 237, 257, 309, 312, 314, 315, 316, 318, 323, 324, 325, 326, and 335 (numbering according to SEQ ID NO: 1). In further embodiments, the one or more amino acid mutations are selected from F314A, F314C, F314D, F314E, F314G, F314H, F314I, F314L, F314N, F314Q, F314S, F314T, F314V, F314W, F314Y, A315F, A315G, A315H, A315I, A315M, A315N, A315S, A315T, A315W, A315Y, G316A, G316C, G316D, G316E, G316F, G316H, G316I, G316K, G316L, G316M, G316P, G316Q, G316R, G316S, G316T, G316V, G316W, G316Y, G318I, G318K, G318R, G318W, R323G, R323K, R325D, R325E, R325K, R325L, R325M, R325N, R325Q, R325S, R325W, F147A, F147K, F147R, N148Q, N148T, L158D, L158N, L158S, L158T, L158W, P197H, P197R, P197T, S231F, S231G, S231H, S231K, S231R, S231V, S231Y, T233F, T233H, T233K, T233R, T233W, T233Y, R237A, R237C, R237D, R237E, R237F, R237G, R237H, R237I, R237K, R237L, R237M, R237N, R237P, R237Q, R237S, R237T, R237V, R237W, R237Y, and D257K.

In some embodiments, the Bxb1 recombinase variant mediates increased transgene insertion at an endogenous target site in a eukaryotic genome, relative to wild-type Bxb1 recombinase.

In other aspects, the present disclosure provides a non-naturally occurring variant of φC31 integrase, Pa557 recombinase, or Pa570 recombinase, comprising one or more amino acid mutations (e.g., substitutions, deletions, or insertions) that produce altered DNA target specificity relative to the wildtype counterpart.

In some embodiments, the φC31 integrase variants comprise one or more amino acid mutations at one or more of positions 273, 275, 279, 375, 376, 377, 379, and 386 (numbering according to SEQ ID NO: 77). In further embodiments, the one or more amino acid mutations are selected from D273, A275, R279, K375, R376, G377, E379, and R386.

In some embodiments, the Pa557 recombinase variants comprise one or more amino acid mutations at one or more of positions 236, 238, 242, 327, 328, 329, 331, and 338 (numbering according to SEQ ID NO: 78). In further embodiments, the one or more amino acid mutations are selected from G236, A238, A242, R327, T328, G329, G331, and R338.

In some embodiments, the Pa570 recombinase variants comprise one or more amino acid mutations at one or more of positions 243, 245, 249, 333, 334, 335, 337, and 344 (numbering according to SEQ ID NO: 79). In further embodiments, the one or more amino acid mutations are selected from E243, S245, K249, 1333, N334, P335, 1337, and Q344.

In some embodiments, the DNA target is specific to a particular allele of a gene.

In some embodiments, the DNA target is in a cell, such as a eukaryotic cell, e.g., a mammalian cell (e.g., a human cell).

Also provided herein are nucleic acid molecules encoding the recombinase variants herein and expression vectors comprising the coding sequences. The vector may be, e.g., a plasmid or a viral vector (e.g., an adeno-associated viral vector, an adenoviral vector, or a lentiviral vector).

The present disclosure also provides a system for editing DNA in a cell, comprising the recombinase variant, the nucleic acid molecule, or the vector herein. In some embodiments, the system further comprises donor DNA, such as a circularized DNA or a linear DNA. IN further embodiments, the donor DNA comprises a sequence selected from SED ID NOs: 80-81 and 134-136 and the sequences found in Tables 3, 4 and 5. The donor DNA may be delivered through, e.g., a plasmid or viral vector.

The editing by the system herein may comprise integration of DNA into the genome of the cell, excision or inversion of DNA in the genome of the cell, or a chromosomal translocation in the genome of the cell. In some embodiments, the editing occurs at a genomic region comprising a sequence selected from SED ID NOs: 80-81 and 134-136 and the sequences found in Tables 3, 4 and 5.

In another aspect, the present disclosure provides a method of editing the genome of a cell, the method comprising providing to the cell the gene editing system herein. In some embodiments, more than one genomic region is edited. The editing method may result in excising DNA from the genome, inverting DNA in the genome, chromosomal swap, recombinase-mediated cassette exchange (RMCE), and/or integrating donor DNA into the genome.

Provided herein are also cells comprising the present system, cells edited by the present methods, or descendent cells thereof. The cells may be eukaryotic cells (e.g., mammalian such as human cells).

In another aspect, the present disclosure provides a method of treating a disease in a subject in need thereof, comprising administering the edited cells to the subject. Also included are cells for use in treating a disease in a subject in need thereof, and use of the cells in the manufacture of a medicament for treating a disease in a subject in need thereof.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a schematic illustrating (A) the structure of Bxb1 recombinase and (B) its mechanism of action as a tetramer on bound attB and attP sites (B. NTD: catalytic N-Terminal domain (NTD); αE: conserved alpha helix; CTD: C-terminal domain; RD: recombinase domain; CC: a coiled-coil motif; and ZD: Zinc ribbon domain (ZD)). The numbers underneath the protein structure in (A) indicate the approximate distance, from the N-terminus of the protein: of the C-terminal border of the NTD (˜150 amino acids), the N-terminal border of the CTD (300 amino acids), and the C-terminus of the CTD (600 amino acids).

FIG. 2 is a schematic illustrating the nucleotide numbering system for attB and attP sequences, including the nucleotides bound by the ZD and RD domains of Bxb1 recombinase. The attP sequence shown is identical to the wildtype sequence (SEQ ID NO: 3), while the attB sequence was edited to be more symmetric compared to the wildtype attB site (SEQ ID NO: 80).

FIGS. 3A-E are a panel of schematics illustrating recombinase-mediated (A) integration, (B) excision, (C) inversion, (D) chromosome swap, and (E) cassette exchange. For recombinase-mediate cassette exchange (RMCE), the donor DNA can be circular (illustrated) or linear (not illustrated).

FIG. 4 is a schematic illustrating the plasmid-based recombination assay used with NGS to determine the DNA sequence specificity of mutant Bxb1 recombinase variants. One plasmid is shown in light gray and the other in black. White and dark gray boxes indicate attB and attP sites, respectively. P1 and P2 refer to primer binding locations. Shown at bottom are the products resulting from successful integration events.

FIG. 5 is a schematic illustrating the chromosomal recombination assay in human K562 cells. Bxb1 facilitates the targeted integration (TI) of a donor plasmid into a chromosomal Bxb1 attB pseudo-site (an endogenous human sequence with some homology to the natural Bxb1 attB target site). White and dark gray boxes indicate attB and attP sites, respectively. F-Primer and R-Primer refer to primer binding locations used for a PCR-based NGS assay to quantify TI events. Shown at bottom is the product resulting from a successful TI event.

FIGS. 6A-C illustrate the alteration of the target site preference of Bxb1 variant S231F. (A) Target preference shift for Bxb1 variant S231F obtained from the plasmid-based experimental system shown in FIG. 4. S231F shows improved targeting of both C and T vs. wild-type (WT) Bxb1 at position 10. (B) Endogenous human Bxb1 target sites s5-8 (SEQ ID NOs: 141 and 264), s5-1 (SEQ ID NOs: 137 and 265), s5-11 (SEQ ID NOs: 143 and 266), and s1-41 (SEQ ID NOs: 161 and 267) with both strands of DNA shown, center dinucleotide shaded gray, RD motifs underlined and base at position 10 of both left and right half site shown in bold. Changes from the A base preferred by WT Bxb1 are indicated by arrows. (C) Targeted integration (TI) values (percent of endogenous alleles containing targeted integration) at these endogenous sites in human cells for either a 50% mixture of the S231F Bxb1 variant and WT Bxb1 or 100% WT Bxb1. Any changes at position 10 of the target site relative to the preferred target of WT Bxb1 are indicated in the second column. Multiple replicates of the WT Bxb1 were performed. Data for WT Bxb1 is the mean value of all replicates +/−the standard deviation. The last column shows the ratio of TI with the S231F variant to TI with WT Bxb1. The increased TI activity for the S231F variant at endogenous sites s5-1, s5-11, and s1-41 is consistent with both the target preference changes observed in the plasmid-based experimental system and the targeting rules shown in Table 1.

FIG. 7 is a list of Bxb1 variants that improve targeted integration (TI) activity vs. wild-type (WT) Bxb1 at endogenous human target sites consistent with the alterations in DNA target sequence shown in Table 1. Variants that gave TI values at least 3 standard deviations above the mean value for WT Bxb1 at the same target in a side-by-side experiment were considered to be improved vs. WT Bxb1. TI and variant/WT data is presented as in FIG. 6C. Sequence alterations at these sites that match alterations in Table 1 are shown in the second, third, and fourth column, where the second column shows the relevant position in the target site of the alteration, the third column shows the alterations at the given position in the left half-site of the target site listed in the first column, and the fourth column shows the alteration in the target site at the relevant position of the right half-site. If the base at the indicated position of the indicated half-site matches the target preferences of WT Bxb1 (e.g. A at position 10, C at position 9, or A at position 7), then the base is indicated as “WT Bxb1.”

FIGS. 8A-C illustrates alterations in target site preference of Bxb1 variants F314G and G316Y. (A) Target preference specificity shift for Bxb1 variant F314G at position 19 (left panel) and target preference specificity shift for Bxb1 variant G316Y at position 21 (right panel) obtained from the plasmid-based experimental system shown in FIG. 4. The bases are numbered according to the scheme shown in FIG. 2. Note that these endogenous human target sites all resemble attB sites. Because attP sites have five bases inserted relative to attB sequences and those inserted bases are at positions 13-17 if present, the base labeled as position 18 is adjacent to the base labeled as position 12. (B) The symmetric attB site (SEQ ID NOs: 268 and 269) used in the plasmid-based experiment system shown in FIG. 4 compared with endogenous human Bxb1 target sites s5-16 (SEQ ID NOs: 146 and 270) and s3-28 (SEQ ID NOs: 163 and 271). The center dinucleotide is shaded in gray and the ZD motifs in each half-site are underlined. Note that the ZD motif in the left half-site of site s5-16 diverges so much from the ZD motif used in the plasmid-based system that it likely represents a distinct sequence motif and cannot be treated as the same ZD sequence motif characterized in the plasmid-based system; thus, it likely does not follow the same target preference rules shown in Table 1. Site s5-16 has a ZD motif similar to the ZD motif in the plasmid-based system in the right half-site; the T in position 19 in the right half-site of this site is shown in bold and indicated by an arrow. Both half-sites of s3-28 contain a ZD motif that resembles the ZD motif used in the plasmid based system and thus ZD motifs in both half-sites are underlined. The G at position 21 of the right half-site is shown in bold and indicated by an arrow. (C) Comparison of targeted integration (TI) data in human cells at the endogenous human target site s5-16 with a 50% mixture of Bxb1 variant F314G and wild-type (WT) Bxb1 vs. 100% WT Bxb1 (top panel), and comparison of TI data in human cells at the endogenous human target site s3-28 with a 50% mixture of Bxb1 variant G316Y and WT Bxb1 vs. 100% WT Bxb1 (bottom panel). Target alterations and TI data is displayed as in FIG. 6C.

FIG. 9 is a list of Bxb1 variants that improve targeted integration (TI) activity vs. wild-type (WT) Bxb1 at the indicated endogenous human target sites. Bases that match the target preferences of WT Bxb1 (e.g., G at position 19) are indicated with “WT Bxb1.” Half-sites with ZD domains that diverge so much from the ZD motif characterized in the plasmid system that they likely represent different sequence motifs are indicated by a blank space in the entry for the relevant half-site. Data is presented as in FIGS. 6C, 7, and 8C.

FIG. 10 is a list of Bxb1 variants that improve targeted integration (TI) activity vs. wild-type (WT) Bxb1 at the indicated endogenous human target sites, with alterations at position 21, 22, 23, or 24 in the ZD motif of one or both half-sites. Target site alterations and data are displayed as in FIG. 9. Target sequence preference for WT Bxb1 is T at positions 21 and 22, and G at positions 23 and 24.

FIG. 11 shows data demonstrating improvement in targeted integration (TI) activity with the D257K Bxb1 variant at a variety of different endogenous human target sites. D257K improved TI activity vs. wild-type (WT) Bxb1 both as a 50% mixture with WT Bxb1 and as 100% D257K variant without any WT Bxb1. In these examples, 100% D257K variant has higher activity than either 50% D257K or 100% WT Bxb1. Thus, D257K appears to represent a Bxb1 variant that can increase activity at most or all endogenous target sites regardless of the exact target site alterations vs. the preferred target site for WT Bxb1.

### DETAILED DESCRIPTION OF THE INVENTION

The present disclosure provides large serine recombinase variants, such as Bxb1 recombinase variants and orthologous recombinase variants, and systems comprising said variants for gene editing. These recombinase variants have altered DNA target sequences compared to their wildtype counterparts and can be used to target endogenous DNA sequences within the genome of organisms of interest, including humans. These enzymes can integrate donor DNA into the genome or excise or invert a target genomic sequence. Thus, they can be used to integrate therapeutic genes into the genome or to repair or remove pathogenic genes from the genome, to achieve therapeutic effects.

The present gene editing systems comprising the recombinase variants are advantageous over other gene editing systems in several important ways. First, the present systems avoid undesired changes to the genome. The most widely used gene editing systems are CRISPR/Cas (clustered regularly interspaced short palindromic repeats (CRISPR) and CRISPR-associated protein (Cas)) systems, zinc-finger nuclease (ZFN) systems, and transcription activator-like effector nuclease (TALEN) systems. Those systems depend upon the activity of nucleases and DNA repair mechanisms, such as homologous recombination and non-homologous end joining, which are error prone and may introduce insertions or deletions (indels) and/or translocations. By having both the cutting and ligating functions, the present recombinases cleave DNA and ligate its breaks at a highly precise, site-specific manner, avoiding the introduction of harmful indels into the genome. Second, there is no inherent size limit to the DNA that can be integrated into a host genome using the present editing systems. Third, the recombination generated by the present systems is not easily reversible without an accessory protein called recombination directionality factor (RDF); thus, the gene edits are stable and heritable. In sum, the present recombinase variants can be used to stably and precisely remove DNA from, integrate large synthetic and/or exogenous donor DNA into, or invert a segment of, a host genome at sites that are specifically recognized by the present recombinase variants.

### I. Variant Serine Recombinase Systems

Provided in the present disclosure are systems for gene editing comprising large serine recombinase variants, such as Bxb1 recombinase variants or orthologous recombinase variants, and optionally donor DNA to be integrated into a host genome at target sites. Also provided are expression constructs for delivering any of the above components, as well as methods for gene editing using one or more of the above components. Each component of the present gene editing system is further described in detail below.

A. Large Serine Recombinase Variants with Altered DNA Target Sequences

As used herein, the term “recombinase” refers to a protein that catalyzes recombination. The term “recombination” refers to the excision, inversion, integration, chromosomal swap, RMCE, or rearrangement of DNA in a target DNA sequence, such as a host genome. The terms “large serine recombinase” and “serine recombinase” refer to a family of recombinase proteins that induce double-stranded DNA breaks and utilize serine residues in their active sites to bind separate DNA segments during recombination.

The term “Bxb1 recombinase” as used herein, refers to a serine recombinase, with an exemplary amino acid sequence shown below:

```
Wildtype Bxb1
(SEQ ID NO: 1)
                10 20 30 40
MRALVVIRLS RVTDATTSPE RQLESCQQLC AQRGWDVVGV
                50 60 70 80
AEDLDVSGAV DPFDRKRRPN LARWLAFEEQ PFDVIVAYRV
                90 100 110 120
DRLTRSIRHL QQLVHWAEDH KKLVVSATEA HFDTTTPFAA
              130 140 150 160
VVIALMGTVA QMELEAIKER NRSAAHFNIR AGKYRGSLPP
              170 180 190 200
WGYLPTRVDG EWRLVPDPVQ RERILEVYHR VVDNHEPLHL
              210 220 230 240
VAHDLNRRGV LSPKDYFAQL QGREPQGREW SATALKRSMI
              250 260 270 280
SEAMLGYATL NGKTVRDDDG APLVRAEPIL TREQLEALRA
              290 300 310 320
ELVKTSRAKP AVSTPSLLLR VLFCAVCGEP AYKFAGGGRK
              330 340 350 360
HPRYRCRSMG FPKHCGNGTV AMAEWDAFCE EQVLDLLGDA
              370 380 390 400
ERLEKVWVAG SDSAVELAEV NAELVDLTSL IGSPAYRAGS
              410 420 430 440
PQREALDARI AALAARQEEL EGLEARPSGW EWRETGQRFG
              450 460 470 480
DWWREQDTAA KNTWLRSMNV RLTFDVRGGL TRTIDFGDLQ
              490 500
EYEQHLRLGS VVERLHTGMS
```

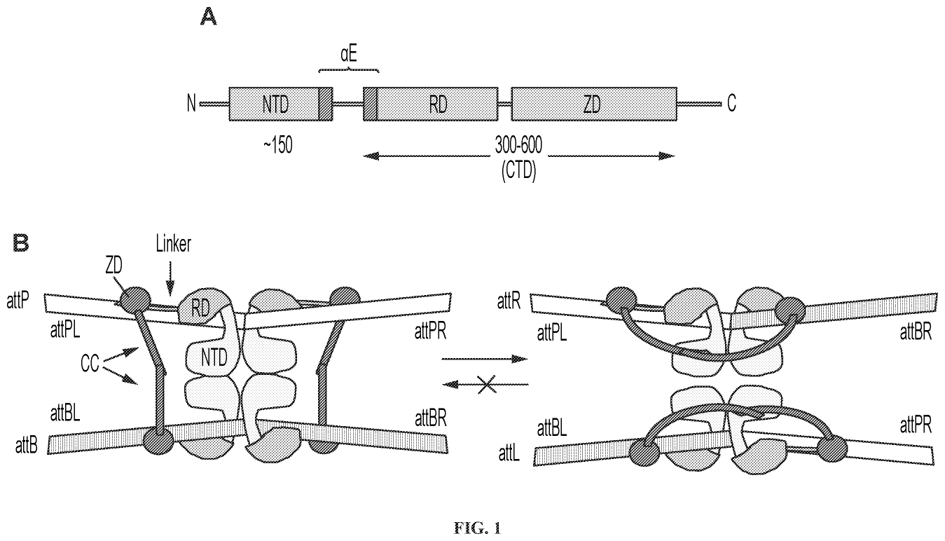

The domain structure of Bxb1 recombinase and its mechanism of action are illustrated in FIG. 1. The following sequences are the attB and attP sites recognized by wildtype Bxb1 recombinase, where the center dinucleotides are italicized and in boldface, the single-underlined regions are recognized by the ZD domains, and the double-underlined regions are recognized by the RD domains:

Wildtype attB
(SEQ ID NO: 2)
GGCTTGTCGACGACGGCGGTCTCCGTCGTCAGGATCAT
Wildtype attP
(SEQ ID NO: 3)
GGTTTGTCTGGTCAACCACCGCGGTCTCAGTGGTGTACGGTACAAACC

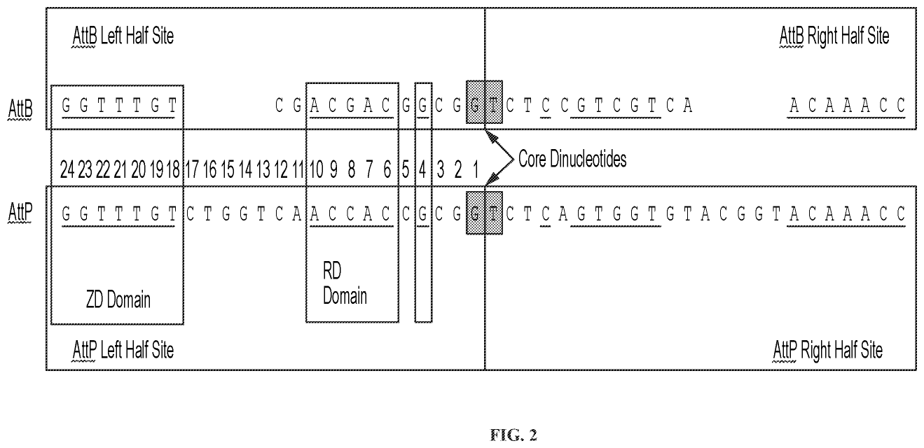

To simplify the search for Bxb1 recombinase variants with altered target DNA specificity, a more symmetric version of the attB target site may be used, with an exemplary nucleotide sequence shown below (SEQ ID NO: 80; see also FIG. 2). The center dinucleotides are italicized and bold, the single-underlined regions are recognized by the ZD domains, and the double-underlined regions are recognized by the RD binding domains:

Symmetric attB
(SEQ ID NO: 80)
GGTTTGTCGACGACGGCGGTCTCCGTCGTCAACAAACC

However, the variants identified through this attB sequence will not be limited to recognizing symmetric attB sequences.

The present disclosure provides variants of the above SEQ ID NO: 1 that have altered DNA recognition sequences. As used herein, the term “Bxb1 recombinase variant(s),” “variant Bxb1 recombinase(s),” “Bxb1 variant(s),” or “variant Bxb1” refers to Bxb1 recombinases with one or more amino acid mutations (e.g., substitutions, insertions, and/or deletions) that alter the DNA targeting specificity of the variant relative to the wildtype (WT) Bxb1 recombinase. The term “orthologous recombinase variant(s)” or “orthologous serine recombinase variant(s)” as used herein refers to orthologous serine recombinases with one or more amino acid mutations that alter the DNA targeting specificity of the variants relative to their WT counterparts.

Serine recombinases, including the present recombinase variants, comprise a catalytic NTD and a CTD involved in sequence-specific DNA recognition. The CTD is further divided into a RD and a ZD containing a CC motif (FIG. 1). The NTD domain may approximately correspond to amino acids 1-145 (numbering according to SEQ ID NO: 1) in wildtype Bxb1 recombinase or the functionally analogous sequence in Bxb1 recombinase variants, or the functionally analogous sequence in orthologous serine recombinase(s) and their variants. The term “functionally analogous sequence” refers to a sequence of amino acid residues or a protein domain having the same or substantially the same biological function. The RD domain may approximately correspond to amino acids 140-287 (numbering according to SEQ ID NO: 1) in wildtype Bxb1 recombinase or the functionally analogous sequence in Bxb1 recombinase variants, or the functionally analogous sequence in orthologous serine recombinase(s) and their variants. The ZD domain may approximately correspond to amino acids 302-500 (numbering according to SEQ ID NO: 1) in wildtype Bxb1 recombinase or the functionally analogous sequences in Bxb1 recombinase variants, or the functionally analogous sequence(s) in orthologous serine recombinase(s) and their variants. The coiled-coil (CC) motif may approximately correspond to that found within the ZD domain in wildtype Bxb1 recombinase or the functionally analogous sequences in Bxb1 recombinase variants, or the functionally analogous sequence(s) in orthologous serine recombinase(s) and their variants.

In some embodiments, the provided recombinase variants may comprise the amino acid sequence GSGSGSHHHHHHGSGPKKKRKV (SEQ ID NO: 249) at the C terminus end, or analogous linkers, His tags, and/or nuclear localization sequences. In some embodiments, the provided recombinase variants comprise a nuclear localization signal at the N- or C-terminal ends.

In some embodiments, the present Bxb1 recombinase variants may comprise mutations within the ZD domain or RD domain amino acid sequences. For example, substitutions in the ZD domain can be made at one or more of amino acids 311-335. Substitutions for the RD domain can be made at one or more of amino acids 137 to 160, 195-201, 229-239, 249-252, and 284-287. The region between the ZD and RD is thought to function as a linker between these domains so substitutions to this linker region can be made at one or more of amino acids 288-301. In some embodiments, the present recombinase variants may comprise one or more mutations at the following amino acid locations: 147, 148, 149, 154, 155, 156, 158, 197, 198, 230, 231, 232, 233, 237, 257, 309, 312, 314, 315, 316, 318, 323, 324, 325, 326, and 335. Unless otherwise indicated, the amino acid positions in Bxb1 recombinase in the present disclosure are numbered in accordance with SEQ ID NO: 1.

In further embodiments, the Bxb1 variant ZD domain may comprise one or more of the following amino acid substitutions:
F314: to A, C, D, E, G, H, I, L, N, Q, S, T, V, W, or Y;
A315: to F, G, H, I, M, N, S, T, W, or Y;
G316: to A, C, D, E, F, H, I, K, L, M, P, Q, R, S, T, V, W, or Y;
G318: to I, K, R, or W;
R323: to G or K; and
R325: to D, E, K, L, M, N, Q, S, or W.

In further embodiments, the Bxb1 variant RD domain may comprise any one or more of the following amino acid mutations:
F147: to A, K, or R;
N148: to Q or T;
L158: to D, N, S, T, or W;
P197: to H, R, or T;
S231: to F, G, H, K, R, V, or Y;
T233: to F, H, K, R, W, or Y; and
R237: to A, C, D, E, F, G, H, I, K, L, M, N, P, Q, S, T, V, W, or Y.

In some embodiments, the present recombinase variants may comprise mutations within the NTD domain or CC coiled motif amino acid sequences.

The effects of exemplary single amino acid substitutions of Bxb1 recombinase (SEQ ID NO: 1) on the recombinase's recognition site are shown in Table 1 and Table 2 below. In these tables, “WT Nt” refers to the nucleotide in the wildtype attB or attP site (e.g., SEQ ID NOs: 2 and 3). Table 1 shows the results of saturation mutagenesis studies at amino acids 147, 148, 149, 154, 155, 156, 158, 197, 198, 230, 231, 232, 233, 237, 257, 309, 312, 314, 315, 316, 318, 323, 324, 325, 326, and 335 (see Example 3 below). In the table, “new Nt” refers to the nucleotide for which the Bxb1 recombinase mutation produces at least a 3-fold change in the nucleotide preference of the wildtype Bxb1 recombinase or shifts the preferred nucleotide of the wildtype Bxb1 recombinase to a different nucleotide. For example, at nucleotide 6 of the WT attB or attP site, when the Bxb1 recombinase variant has a F147A, N148T, L158W, T233F, or T233Y substitution, the Bxb1 recombinase variant has at least a 3-fold higher binding affinity for nucleotide A compared to the wildtype Bxb1 recombinase.

TABLE 1
Bxb1 Recombinase Mutants with Altered DNA Target Sequences

| Pos.* | WT Nt | New Nt | Mutants |
|---|---|---|---|
| 6 | C | A | F147A, N148T, L158W, T233F, T233Y |
| | | G | F147A, F147R, N148Q, N148T, P197H |
| 7 | A | C | F147K, F147R, N148Q, N148T, L158D, L158N, L158S, |
| | | | L158T, T233F, T233Y |
| 9 | C | A | N148T, R237A, R237C, R237F, R237G, R237H, R237I, |
| | | | R237K, R237L, R237M, R237N, R237Q, R237S, R237V, |
| | | | R237W, R237Y |
| | | G | F147A, F147R, N148T, P197H, P197R, P197T, R237A, |
| | | | R237C, R237D, R237E, R237F, R237G, R237H, R237I, |
| | | | R237M, R237N, R237P, R237Q, R237S, R237T, R237V, |
| | | | R237W, R237Y |
| | | T | R237A, R237C, R237H, R237M, R237N, R237S, R237T, |
| | | | R237V |
| 10 | A | C | F147A, F147R, N148Q, N148T, S231F, S231G, S231H, |
| | | | S231K, S231R, S231V, S231Y, T233H, T233K, T233W |
| | | G | T233R |
| | | T | N148Q, N148T, P197H, S231F, S231G, S231R, S231V, |
| | | | T233F, T233W, T233Y |
| 18 | T | A | R325W |
| | | C | R325K |
| 19 | G | A | F314D, F314Q, A315T |
| | | C | F314L, A315F, A315I, A315M, A315T, A315W, A315Y, |
| | | | G318R, R325K |
| | | T | F314A, F314C, F314G, F314N, F314Q, F314S, F314T, |
| | | | A315F, A315I, A315S, A315W, A315Y, G318R |
| 20 | T | A | G318R, R325K, R325W |
| | | C | A315F, A315G, A315W, A315Y, R325D, R325E, R325K, |
| | | | R325L, R325M, R325N, R325Q, R325S |
| | | G | F314D, F314N, F314S, F314T, A315T, G318K, G318R |
| 21 | T | A | F314D, F314I, F314L, A315F, A315W, A315Y |
| | | C | F314D, F314E, R323G, R325W |
| | | G | F314A, F314C, F314H, F314N, F314S, F314T, F314V, |
| | | | F314W, G316F, G316H, G316K, G316P, G316Y |
| 22 | T | A | F314T, A315F, A315W, A315Y, G316C, G316S, G318I |
| | | G | F314D, F314T, F314Y, A315F, A315H, A315N, A315W, |
| | | | A315Y, G316A, G316H, G316Q, G316R, G316S, R323K |
| 23 | G | A | A315F, A315H, A315W, A315Y, G316T, G316V, G316W, |
| | | | G318I, G318K, G318R |
| | | C | A315F, A315H, A315N, A315Y, G316A, G316C, G316D, |
| | | | G316E, G316F, G316H, G316I, G316L, G316M, G316Q, |
| | | | G316S, G316T, G316V, G316W, G316Y |
| | | T | G316C, G316I, G316V, G316H |
| 24 | G | C | A315F, A315Y, G316W, G318W |

*Nucleotide position in the recombinase recognition site corresponding to that in FIG. 2.

In some embodiments, the Bxb1 recombinase variant may comprise two or more substitutions, e.g., those shown in Table 1 above. Such a variant may have a higher binding affinity to a target sequence that contains two or more nucleotide differences from the WT attB or attP recognition sequence. For example, a Bxb1 recombinase variant that comprises mutations R237K and T233R will have a higher binding affinity to the target sequence that comprises A at position 9 and G at position 10. Some of the single substitutions will produce increases in binding affinities at multiple nucleotide positions. By way of example, a Bxb1 recombinase variant that comprises the mutation F147R will have a higher binding affinity to the target sequence that comprises G at position 6, C at position 7, G at position 9, and C at position 10. Some of the substitutions will produce increases in binding affinities for multiple nucleotides at a single position. For example, a Bxb1 recombinase variant that comprises the mutation R237V will have higher binding affinities to the target sequences that comprise A, G, or T at position 9.

In certain embodiments, the Bxb1 recombinase variants may comprise one or more substitutions, e.g., those shown in Table 2 below. Shown in Table 2 are the two Bxb1 recombinase mutations that produce in the targeted DNA binding sequence the greatest increase in binding affinity for A, C, G, or T at the indicated nucleotide position (position numbers according to FIG. 2). For example, while the mutations F147A, N148T, L158W, T233F, and T233Y produce a Bxb1 recombinase variant with increased binding affinity to the target sequence that comprises A at position 6, T233F and N148T produce the greatest increase in affinity. The underlined mutations produce Bxb1 recombinase variants that recognize a target sequence comprising a new preferred nucleotide at the indicated position. For example, Bxb1 recombinase variants with a T233R mutation preferentially recognize sequences with a Gat position 10, while the wildtype Bxb1 recombinase preferentially recognizes sequences with an A at position 10.

TABLE 2
Bxb1 Recombinase Mutants with Particular Increases in Binding Affinity

| Domain | Pos.* | WT Nt | A | C | G | T |
|---|---|---|---|---|---|---|
| RD | 6 | C | T233F, | WT Bxb1 | N148T, | |
| | | | N148T | | F147R | |
| | 7 | A | WT Bxb1 | N148T, | | |
| | | | | T233Y | | |
| | 9 | C | R237G/K | WT Bxb1 | R237D/E | R237A/T |
| | 10 | A | WT Bxb1 | S231R/Y | T233R | T233Y, |
| | | | | | | S231R |
| ZD | 18 | T | R325W | R325K | | WT Bxb1 |
| | 19 | G | F314D/Q | R325K, | WT Bxb1 | F314G/S |
| | | | | F314L | | |
| | 20 | T | R325K, | R325Q/M | F314T/D | WT Bxb1 |
| | | | G318R | | | |
| | 21 | T | F314L/I | F314E/D | F314N/V | WT Bxb1 |
| | 22 | T | G316C/S | | F314Y, | WT Bxb1 |
| | | | | | G316R | |
| | 23 | G | G316W, | G316Y/D | WT Bxb1 | G316C/I |
| | | | A315Y | | | |
| | 24 | G | | G318W, | WT Bxb1 | G316H |
| | | | | G316W | | |

*Nucleotide position in the recombinase recognition site corresponding to that in FIG. 2.

In some aspects of the present disclosure, a recombinase orthologous to the Bxb1 recombinase may also be used. In some embodiments, the orthologous serine recombinase is φC31 integrase, comprising an exemplary amino acid sequence below:

```
Wildtype φC31
(SEQ ID NO: 77)
                10 20 30 40
MDTYAGAYDR QSRERENSSA ASPATQRSAN EDKAADLQRE
                50 60 70 80
VERDGGRFRF VGHFSEAPGT SAFGTAERPE FERILNECRA
                90 100 110 120
GRLNMIIVYD VSRFSRLKVM DAIPIVSELL ALGVTIVSTQ
              130 140 150 160
EGVFRQGNVM DLIHLIMRLD ASHKESSLKS AKILDTKNLQ
              170 180 190 200
RELGGYVGGK APYGFELVSE TKEITRNGRM VNVVINKLAH
              210 220 230 240
STTPLTGPFE FEPDVIRWWW REIKTHKHLP FKPGSQAAIH
              250 260 270 280
PGSITGLCKR MDADAVPTRG ETIGKKTASS AWDPATVMRI
              290 300 310 320
LRDPRIAGFA AEVIYKKKPD GTPTTKIEGY RIQRDPITLR
              330 340 350 360
PVELDCGPII EPAEWYELQA WLDGRGRGKG LSRGQAILSA
              370 380 390 400
MDKLYCECGA VMTSKRGEES IKDSYRCRRR KVVDPSAPGQ
              410 420 430 440
HEGTCNVSMA ALDKFVAERI FNKIRHAEGD EETLALLWEA
              450 460 470 480
ARRFGKLTEA PEKSGERANL VAERADALNA LEELYEDRAA
              490 500 510 520
GAYDGPVGRK HFRKQQAALT LRQQGAEERL AELEAAEAPK
              530 540 550 560
LPLDQWFPED ADADPTGPKS WWGRASVDDK RVFVGLFVDK
              570 580 590 600
IVVTKSTTGR GQGTPIEKRA SITWAKPPTD DDEDDAQDGT
EDVAA
```

In some embodiments, the orthologous serine recombinase is Pa557 recombinase, comprising an exemplary amino acid sequence below:

```
Wildtype Pa557
(SEQ ID NO: 78)
                10 20 30 40
MNMHSPTVTT RAALYLRVST ARQAEHDISI PDQKRQGEAY
                50 60 70 80
CEQRGFQLVE TYVEPGATAT NDKRPEFQRM IEAGTSKPAP
                90 100 110 120
FDIVVVHSFS RFFRDHFEME FYVRKLAKNG VKLVSITQEM
              130 140 150 160
GDDPMHQMMR QIMALFDEYQ SKENAKHVLR AMNENARQGF
              170 180 190 200
WNGARPPIGY RIVAAEQRGS KTKKKLEIDP LHADTVRLIY
              210 220 230 240
RLFLEGDGTR GAMGVKAIAT YLNERRFFTR DGGRWGLAQI
              250 260 270 280
HAILTRTTYI GEHRFNTRSH KDREKKPESE IAIMAVPPLI
              290 300 310 320
EREIYDAVQA RLKSRNPMVT PARVSSGPTL LTGICFCAKC
              330 340 350 360
GGAMTLRTGQ GSTGATYRYY TCSTKARQGK TGCKGRTIPM
              370 380 390 400
DKLDHLVADH IGDRLLQPKR LETVLASVID RRQERAERRR
              410 420 430 440
EHLAELNRRI TEADQRLGRL FDAIEAGMVD KDDAMAKERM
              450 460 470 480
VSLKALRDQA AADAERTQLA LDSSGNQGVS PDMLKGFARK
              490 500 510 520
ARERIRLDDG GYRRDHLRAL AQRVEVADDE VRIMGSKSEL
              530 540 550
LRTLVAASSV ETAAFGVQSS VLKWRTQEDS NLRPLGS
```

In some embodiments, the orthologous serine recombinase is Pa570 recombinase, comprising an exemplary amino acid sequence below:

```
Wildtype Pa570
(SEQ ID NO: 79)
                10 20 30 40
MARLISYLRM STSEQLRGFS LERQRKLIAD FAAKNGLSVE
                50 60 70 80
EENTLEDIGR SSFSDDAQQK ELTRFFENLN AGKYEPGDVF
                90 100 110 120
ALENIDRLTR RGPVDAILKV NQIISKGLKL AIISGNEQRI
              130 140 150 160
IEDVNDVFTI INLSIDASRA NKESKNKSDK GLSNWQEKRN
              170 180 190 200
LASTYKIAMT AQAPAWLDTE IFYIFDEEKK KNTKRRKYVL
              210 220 230 240
NEEKAEAVRL IFDLYSNGNG ALKIKNILNE RNIPTFKGAP
              250 260 270 280
YWEPSIITKI LKNPATFGLY QPKKQGTGKR DLIAAGEPIN
              290 300 310 320
DYFPPVITRD LFEQCEHIRE GNSTRKGRKG KLFTNLFTGL
              330 340 350 360
LTCSKCGGPV HLINPGIDKR NKVQKSIYYL VCKRAKFTKE
              370 380 390 400
CTTKRVRYDD FEIALLKAIQ EINLADILNE NNPLEILVKK
              410 420 430 440
QRSKETEINK KRKLIENFQR QFLENDGDLP SFMISQAKDA
              450 460 470 480
EISIKELEED QREIASEIAQ LNIYNSNVDN AIEELKENAD
              490 500 510 520
YGTRSKINLL FHEIIKNISL DTENQFYTVR FKNGVMRVIT
              530 540 550 560
AAGFIATTEE QTQADINAIL QSIEGPRIPR EIATDAEKLI
              570
EYLKAREVIE
```

In some embodiments, the orthologous serine recombinase is LI Integrase, comprising an exemplary amino acid sequence below:

```
Wildtype LI
(SEQ ID NO: 131)
                10 20 30 40
MKAAIYIRVS TQEQVENYSI QAQTEKLTAL CRSKDWDVYD
                50 60 70 80
TFIDGGYSGS NMNRPALNEM LSKLHEIDAV VVYRLDRLSR
                90 100 110 120
SQKDTITLIE EYFLKNNVEF VSLSETLDTS SPFGRAMIGI
              130 140 150 160
LSVFAQLERE TIRDRMVMGK IKRIEAGLPL TTAKGRIFGY
              170 180 190 200
DVIDTKLYIN EEEAKQLRLI YDIFEEEQSI TFLQKRLKKL
              210 220 230 240
GFKVRTYNRY NNWLINDLYC GYVSYKDKVH VKGIHEPIIS
              250 260 270 280
EEQFYRVQEI FSRMGKNPNM NKESASLLNN LVVCSKCGLG
              290 300 310 320
FVHRRKDTVS RGKKYHYRYY SCKTYKHTHE LEKCGNKIWR
              330 340 350 360
ADKLEELIID RVNNYSFASR NIDKEDELDS LNEKLKIEHA
              370 380 390 400
KKKRLFDLYI NGSYEVSELD SMMNDIDAQI NYYEAQIEAN
              410 420 430 440
EELKKNKKIQ ENLADLATVD FNSLEFREKQ LYLKSLINKI
              450
YIDGEQVTIE WL
```

In some embodiments, the orthologous serine recombinase is A118 Integrase, comprising an exemplary amino acid sequence below:

```
Wildtype A118
(SEQ ID NO: 132)
                10 20 30 40
MKAAIYIRVS TQEQVENYSI QAQTEKLTAL CRSKDWDVYD
                50 60 70 80
IFIDGGYSGS NMNRPALNEM LSKLHEIDAV VVYRLDRLSR
                90 100 110 120
SQRDTITLIE EYFLKNNVEF VSLSETLDTS SPFGRAMIGI
              130 140 150 160
LSVFAQLERE TIRDRMVMGK IKRIEAGLPL TTAKGRTFGY
              170 180 190 200
DVIDTKLYIN EEEAKQLQLI YDIFEEEQSI TFLQKRLKKL
              210 220 230 240
GFKVRTYNRY NNWLTNDLYC GYVSYKDKVH VKGIHEPIIS
              250 260 270 280
EEQFYRVQEI FTRMGKNPNM NRDSASLLNN LVVCSKCGLG
              290 300 310 320
FVHRRKDTMS RGKKYHYRYY SCKTYKHTHE LEKCGNKIWR
              330 340 350 360
ADKLEELIIN RVNNYSFASR NVDKEDELDS LNEKLKIEHA
              370 380 390 400
KKKRLFDLYI NGSYEVSELD SMMNDIDAQI NYYESQIEAN
              410 420 430 440
EELKKNKKIQ ENLADLATVD FDSLEFREKQ LYLKSLINKI
              450
YIDGEQVTIE WL
```

In some embodiments, the orthologous serine recombinase is TP901 recombinase, comprising an exemplary amino acid sequence below:

```
Wildtype TP901
(SEQ ID NO: 133)
                10 20 30 40
MTKKVAIYTR VSTTNQAEEG FSIDEQIDRL TKYAEAMGWQ
                50 60 70 80
VSDTYTDAGF SGAKLERPAM QRLINDIENK AFDTVLVYKL
                90 100 110 120
DRLSRSVRDT LYLVKDVFTK NKIDFISLNE SIDTSSAMGS
              130 140 150 160
LFLTILSAIN EFERENIKER MTMGKLGRAK SGKSMMWTKT
              170 180 190 200
AFGYYHNRKT GILEIVPLQA TIVEQIFTDY LSGISLTKLR
              210 220 230 240
DKLNESGHIG KDIPWSYRTL RQTLDNPVYC GYIKFKDSLF
              250 260 270 280
EGMHKPIIPY ETYLKVQKEL EERQQQTYER NNNPRPFQAK
              290 300 310 320
YMLSGMARCG YCGAPLKIVL GHKRKDGSRT MKYHCANRFP
              330 340 350 360
RKTKGITVYN DNKKCDSGTY DLSNLENTVI DNLIGFQENN
              370 380 390 400
DSLLKIINGN NQPILDTSSF KKQISQIDKK IQKNSDLYLN
              410 420 430 440
DFITMDELKD RTDSLQAEKK LLKAKISENK FNDSTDVFEL
              450 460 470 480
VKTQLGSIPI NELSYDNKKK IVNNLVSKVD VTADNVDIIF
KFQLA
```

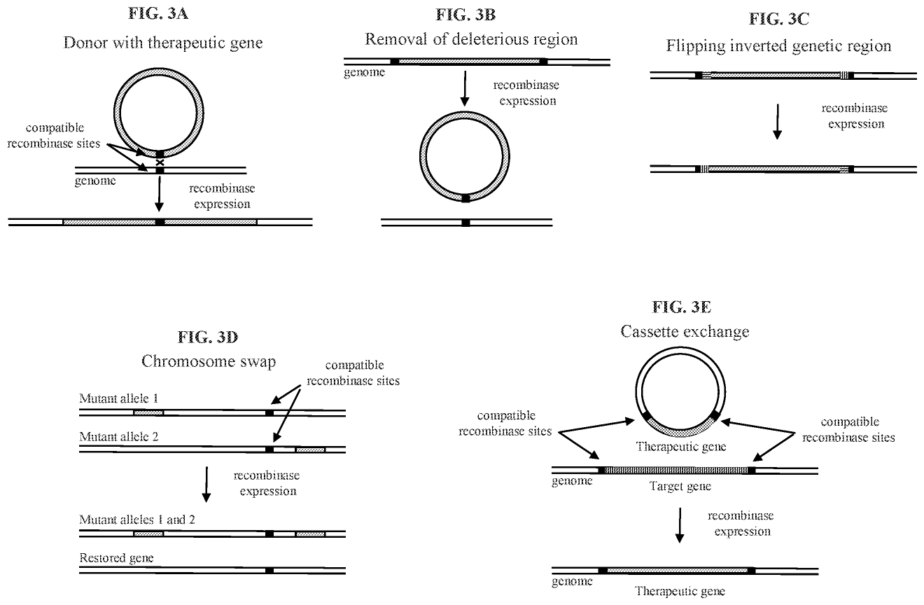

An alignment of some of the above orthologous recombinases can be found in FIG. 3 of Rutherford et al., supra.

In some embodiments, the orthologous recombinase variant may comprise mutations within the ZD or RD. In some embodiments, the orthologous recombinase variant may comprise mutations within the NTD domain or CC coiled motif amino acid sequences. In some embodiments, the orthologous recombinase may comprise one or more mutations at positions that correspond to residues S231, T233, R237, F314, A315, G316, G318, and R325 of Bxb1 recombinase. For example, the φC31 integrase variant may comprise one or more mutations at amino acid positions D273, A275, R279, K375, R376, G377, E379, and R386 (numbering according to SEQ ID NO: 77); the Pa557 recombinase variant may comprise one or more mutations at amino acid positions G236, A238, A242, R327, T328, G329, G331, and R338 (numbering according to SEQ ID NO: 78); and the Pa570 recombinase variant may comprise one or more mutations at amino acid positions E243, S245, K249, 1333, N334, P335, 1337, and Q344 (numbering according to SEQ ID NO: 79).

In some embodiments, the system comprises a mixture of different Bxb1 recombinase variants and/or other orthologous serine recombinase variants, and/or the nucleic acids that encode them, as well as any associated donor DNA molecules.

In some embodiments, the Bxb1 recombinase variant mediates increased transgene insertion at an endogenous target site in a eukaryotic genome, relative to wild-type Bxb1 recombinase. In some embodiments, the Bxb1 recombinase variant increases transgene insertion at least 1-, 2-, 3-, 4-, 5-, 6-, 7-, 8-, 9-, 10-, 11-, 12-, 13-, 14-, 15-, 16-, 17-, 18-, 19-, or 20-fold, relative to wild-type Bxb1 recombinase.

### B. Recombinase Variant Recognition Sites

The present recombinase variants are collectively more versatile than their WT counterparts in that the variants can bind a wider repertoire of DNA recognition sites. The table below (Table 3) shows attB-like sequences that may be more efficiently bound by one or mixtures of the variants herein than by wildtype Bxb1 recombinase. The sequences in the table below are SEQ ID NOs: 83 to 130, in the order of appearance. In the table, underlined nucleotides are center dinucleotides; “hg38 coordinates” refer to positions according to the Genome Reference Consortium Human Build 38 standard; and boldfaced and italicized nucleotides indicate mismatches to the consensus attB sequences below (for sites 1-14, this is SEQ ID NO: 82 (see Examples); for sites 15-24, this is SEQ ID NO: 134; for sites 25-36, this is SEQ ID NO: 135; and for sites 37-48, this is SEQ ID NO: 136):

(SEQ ID NO: 134)
5′-GGTTTGTNNACNACNGNNNNNNCNGTNGTNAGGATCNN
(SEQ ID NO: 135)
5′-NNGATCCTGACGACNGNNNNNNCNGINGINNACAAACC
(SEQ ID NO: 136)
5′-NNGATCCTGACGACNGNNNNNNCNGTNGTNAGGATCNN

TABLE 3
Endogenous Human Binding Sites for Wildtype Bxb1 Recombinase

| SEQ ID NO | # | hg38 coordinates | | Endogenous Human Sequence |
|---|---|---|---|---|
| 83 | 1 | chr18 | 23228819 | TGCTTGTTGCCTACAGCCTCTGCAGAAGTTCACAAACT |
| 84 | 2 | chr21 | 39796718 | GGGTTGTTGACTACAGATTGCACTATTTTGCAGAAACC |
| 85 | 3 | chr5 | 130870404 | TAGTTGTAGACCACAGAAATTTCTGCTGTTCAGAAACT |
| 86 | 4 | chr10 | 114431581 | GTTTTGTGGACAATGGTTTCCTCTGCTGTAAAGAAACG |
| 87 | 5 | chr4 | 78331600 | GGGTTGGTGGCAATGGAGATCTCAGTGGTGAAAAATCA |
| 88 | 6 | chr3 | 21564386 | AGCTTGTTTACATGTGAATTTTCTGTAGTAAACAACCA |
| 89 | 7 | chr17 | 37733272 | TGTTTGTACTCTACCGAAGATTCCATAGTTAACAACCA |
| 90 | 8 | chr7 | 37683631 | TATTTCTTGACAATTGGCCTCTCTGTAGTTTGCAACCT |
| 91 | 9 | chr9 | 94861248 | GGTTTGAGGGCAGCAGCAGCAGCTGCTGTCACCAAACA |
| 92 | 10 | chrX | 50461727 | GTTTTGTTCACTGTAGAATCCTCAGTGGTTAGCACAAT |
| 93 | 11 | chr1 | 203596869 | CTCTTGATGACTGCAGAGTATTCCATTGTTGACAAATC |
| 94 | 12 | chr5 | 35969016 | TGTTTCTCAACTACAGCATGAGCTGTGGTTGACAGTAA |
| 95 | 13 | chr1 | 183975066 | GATTTGGATACACCTGAGATGACAGTTGTCAAGAAATC |
| 96 | 14 | chr6 | 120401383 | ATTTGTTTAACTACTGAGTTTTCAGTAGTTAAAAAGCT |
| 97 | 15 | chr2 | 131113644 | GGCTTTTTCATTACAGAACACCCAGTTGTCAGTATCAG |
| 98 | 16 | chr18 | 68095661 | TTATTGTTGACAACTGATATTTCCACGGTGAGAATCCC |
| 99 | 17 | chr10 | 95438042 | GGGTTTTTAAACACTGACTTGCCTGGTGTGAGGATCCA |
| 100 | 18 | chr11 | 93987223 | AATTTGCTGACAACAGGTGCTTCTGTGGTTAGGAGTGG |
| 101 | 19 | chr18 | 38351472 | GGCTTTTAGACTGATGAGTGATCAGTCTTCAGGATCCC |
| 102 | 20 | chr6 | 84802299 | ATAGTGTTTACCTCAGAAAGATCTGTGGTAAGGACCAA |
| 103 | 21 | chrX | 54472830 | GGTGTGAGGACAAGAGAGTGAGCAGTAGTCAAGAGCTC |
| 104 | 22 | chr3 | 138022040 | TGTATGTGGACCATAGGAAAAGCTGGAGTCAGGAGCTC |
| 105 | 23 | chr7 | 125717751 | TGTTTGTGGAGGAGTGATGGCTCAGTGGTATGGATCTA |
| 106 | 24 | chr6 | 29957229 | GCTTTTTTGACCTCGGCCTCCGCTCAGGTCAGGACCAG |
| 107 | 25 | chr2 | 118918350 | CTGATACTTGCAACTGAACACACTGTAGTCCACACACC |
| 108 | 26 | chr1 | 218729444 | GAGATCTTGGCAATAGGAACCTCAGCTGTCAGCAAGCA |
| 109 | 27 | chr12 | 82686460 | GAGTTTGTGACAACCGAAGCTGCAGCAGTCGCCAAACC |
| 110 | 28 | chr1 | 12564817 | TTGCTTCTCACCGTAGATGACCCTCTTGTCCACAAACC |
| 111 | 29 | chr14 | 33253493 | CAGCTCCTCACAACTGATTTCCCAATGGTAATCAAGCT |
| 112 | 30 | chr5 | 168835161 | GAGATCCTAGCCACAGGCGGGCCCAGGGTTGGCAAACC |
| 113 | 31 | chr1 | 205211683 | TGGCTCCCAACCTCCGTCACTGCCGTTGCAAACAAACC |
| 114 | 32 | chr17 | 51831373 | AGATTCCTGACTACAGTGGATGCTGTTGTTTGCAATTC |
| 115 | 33 | chr7 | 73438826 | CAGCTCCTGAACACAGCAGTCCCAGGTGTTGAAAAGCC |
| 116 | 34 | chr8 | 138569746 | TTGGTCTTTACAACTGGGGCTACTGTGGTGGATAATCG |
| 117 | 35 | chr7 | 26280167 | CAGGTCCTGACCTCAGAGCCAGCTGGAATGAAAAAACC |
| 118 | 36 | chr18 | 64064874 | CAGATGCTCTCCACAGGGTCTGCTGCAGTTTACAATCC |
| 119 | 37 | chr15 | 26397212 | ATGATCCTCACTACAGCTATGGCAATTGTATTGATCCT |
| 120 | 38 | chr11 | 121591710 | GTGCTCCTAACAGCAGGCGGAACAGAGGTGAGGATCCT |
| 121 | 39 | chr1 | 84080800 | AAGATCCTGACCTCCGAAGAAGCTGGAGTTGAGATCAT |
| 122 | 40 | chr6 | 124844046 | ATGGTCCCAGCTACTGGGGAGGCTGAGGTGAGGATCGC |
| 123 | 41 | chr3 | 194380608 | TTGCTCCTCACAGCAGAGGACTCTGAAGACAGGAGCAT |
| 124 | 42 | chr21 | 38902753 | TGGATCTTCACTATGGCACTCCCATTGGTATGGATCTC |
| 125 | 43 | chr18 | 33954365 | CAGATCTATACGGCTGACTACACAGTGGTGAGAACCAT |
| 126 | 44 | chr14 | 42626161 | ATGATCCTCACAGCAGATATATCTGTTCTCAAGATTTA |
| 127 | 45 | chr10 | 27365656 | ATGATCTTGACAACAGGGAGTGCTGGGTTCTGGAACAT |
| 128 | 46 | chr4 | 9830819 | CTGATGCTGACCACAGCAAATGCTGCCAACAGGATCCC |
| 129 | 47 | chr12 | 69787548 | ATGATCCTAACTCTTGCTTTATCTACTGTGAAAATCAT |
| 130 | 48 | chr11 | 97082317 | TTGGTCCTGGATCCTGAGGTTGCTGTGGTGAAGATCCC |

A wildtype Bxb1 recombinase or an ortholog thereof may be engineered as described above by, e.g., adopting the amino acid substitutions shown in Tables 1 and 2, such that the engineered protein can now bind with high affinity to these endogenous genomic target sites in human cells.

C. Donor DNA with Engineered attB or attP Sequences.

The provided recombinase variants can integrate donor DNA (e.g., circularized donor DNA) into a host genome if the donor and host DNA include wildtype or variant attB and/or attP sequences. The present disclosure provides nucleic acid molecules comprising the variant attB and attP sequences recognized by the provided recombinase variants.

In some embodiments, the attB sequences described herein comprise any one of the sequences of SEQ ID NOs: 83-130, as shown in Table 3. In some embodiments, the attB sequences described herein comprise any one of the sequences of SEQ ID NOs: 4-40, as shown in Table 4. In some embodiments, the attP sequences described herein comprise any one of the sequences of SEQ ID NOs: 41-76, as shown in Table 5. In some embodiments, the attB or attP sequences may comprise a sequence selected from SEQ ID NOs: 80-82 and 134-136.

### D. Recombination Events

Bxb1 recombinase can generate a range of dimers that recognize and specifically bind DNA recognition attB and attP sites. When the attB and attP sites are present in the same cell, a pair of Bxb1 recombinase dimers that bind the attB and attP sites form a tetramer, bringing together the DNA segments containing the attB and attP sites and initiating DNA recombination. When the DNA segments are on the same DNA molecule (e.g., chromosome), this recombination event can lead to DNA inversion or deletion. When the DNA segments are on different DNA molecules, this recombination event may lead to DNA integration or cassette exchange. The present recombinase variants contain mutations that enable them to catalyze the recombination between variant attB and attP sequences, greatly expanding the repertoire of recognizable endogenous sites and increasing the versatility of the enzymes.

The present recombinase variants can produce recombination events of interest, such as integration, inversion, excision, chromosome swap, and RMCE. FIG. 3A illustrates integration mediated by the recombinase variants of the present disclosure. Integration requires the presence of donor DNA with an attachment site that is compatible to the attachment site of the target DNA. FIG. 3B illustrates excision mediated by the recombinase variants of the present disclosure. Excision requires two complementary attachment sites similarly orientated on the same DNA molecule. FIG. 3C illustrates inversion mediated by the recombinase variants of the present disclosure. Inversion requires two complementary attachment sites oppositely orientated on the same DNA molecule. FIG. 3D illustrates chromosome swap mediated by the recombinase variants of the present disclosure. Chromosome swap requires two complementary attachment sites similarly orientated on two separate linear DNA molecules. FIG. 3E illustrates RMCE mediated by the recombinase variants of the present disclosure. RMCE requires donor and target DNA molecules each contain two complementary attachment sites that are not cross-compatible. For example, a donor DNA molecule containing gene X flanked by two different attB sites and a target DNA molecule containing gene Y flanked by two different attP sites where the compatible attachment sites upstream of gene X and Y are complementary and the compatible attachment sites downstream of genes X and Y are complementary, but the upstream and downstream sites are not cross-compatible. Cross-compatibility can be avoided, for example, by using different center dinucleotides in the upstream and downstream attachment sites. This system allows genes X and Y to be exchanged in the presence of the recombinase variants of the present disclosure. Only in the presence of the appropriate RDF can serine recombinases bind to attR and attL sites and mediate the reverse recombination event.

### II. Delivery of Variant Recombinase Systems

The Bxb1 recombinase variants or orthologous recombinase variants of the present disclosure may be introduced to target cells as a protein, through a variety of methods (e.g., electroporation, lipid nanoparticles, cationic or anionic liposomes, or a nuclear localization signal (e.g., in combination with liposomes)). In some embodiments, a provided recombinase variant is introduced to target cells through a nucleic acid molecule encoding it, for example, a DNA plasmid or mRNA. The nucleic acid molecule may be in a nucleic acid expression vector, which may include expression control sequences such as promoters, enhancers, transcription signal sequences, and transcription termination sequences that allow expression of the coding sequence for the provided recombinase variants. “Delivery of a system” as described herein may refer to either delivery of a system comprising a Bxb1 recombinase variant or orthologous recombinase variant as described herein or delivery of nucleic acid molecules encoding said system of a provided recombinase variant or vectors or expression constructs comprising said nucleic acid molecules.

In some embodiments, the promoter on the vector for directing a provided recombinase variant's expression is a constitutively active promoter or an inducible promoter. Suitable promoters include, without limitation, a Rous sarcoma virus (RSV) long terminal repeat (LTR) promoter (optionally with an RSV enhancer), a cytomegalovirus (CMV) promoter (optionally with a CMV enhancer), a CMV immediate early promoter, a simian virus 40 (SV40) promoter, a dihydrofolate reductase (DHFR) promoter, a β-actin promoter, a phosphoglycerate kinase (PGK) promoter, an EF1α promoter, a Moloney murine leukemia virus (MoMLV) LTR, a creatine kinase-based (CK6) promoter, a transthyretin promoter (TTR), a thymidine kinase (TK) promoter, a tetracycline responsive promoter (TRE), a hepatitis B Virus (HBV) promoter, a human al-antitrypsin (hAAT) promoter, chimeric liver-specific promoters (LSPs), an E2 factor (E2F) promoter, the human telomerase reverse transcriptase (hTERT) promoter, a CMV enhancer/chicken β-actin/rabbit β-globin promoter (CAG promoter; Niwa et al., Gene (1991) 108 (2): 193-9), and an RU-486-responsive promoter. In addition, the promoter may include one or more self-regulating elements whereby a provided recombinase variant can bind to and repress its own expression level to a preset threshold.

Any method of introducing the nucleotide sequence into a cell may be employed, including but not limited to, electroporation, calcium phosphate precipitation, microinjection, cationic or anionic liposomes, liposomes in combination with a nuclear localization signal, naturally occurring liposomes (e.g., exosomes), or viral transduction. In certain embodiments, the nucleotide sequence is in the form of mRNA and is delivered to a cell via electroporation.

For in vivo delivery of an expression vector, viral transduction may be used. A variety of viral vectors known in the art may be adapted by one of skill in the art for use in the present disclosure, for example, vaccinia vectors, adenoviral vectors, lentiviral vectors, poxyviral vectors, adeno-associated viral (AAV) vectors, retroviral vectors, and hybrid viral vectors. In some embodiments, the viral vector used herein is a recombinant AAV (rAAV) vector. Any suitable AAV serotype may be used. For example, the AAV may be AAV1, AAV2, AAV3, AAV3b, AAV4, AAV5, AAV6, AAV7, AAV8, AAV8.2, AAV9, AAV.PHP.B, AAV.PHP.eB, or AAVrh10, or of a novel serotype or a pseudotype such as AAV2/8, AAV2/5, AAV2/6, AAV2/9, or AAV2/6/9. In some embodiments, the expression vector is an AAV viral vector and is introduced to the target human cell by a recombinant AAV virion whose genome comprises the construct, including having the AAV Inverted Terminal Repeat (ITR) sequences on both ends to allow the production of the AAV virion in a production system such as an insect cell/baculovirus production system or a mammalian cell production system. The AAV may be engineered such that its capsid proteins have reduced immunogenicity or enhanced transduction ability in humans. Viral vectors described herein may be produced using methods known in the art. Any suitable permissive or packaging cell type may be employed to produce the viral particles. For example, mammalian (e.g., 293) or insect (e.g., sf9) cells may be used as the packaging cell line.

Any type of cell may be targeted for the gene editing methods described herein. For example, the cells may be eukaryotic or prokaryotic. In some embodiments, the cells are mammalian (e.g., human) cells or plant cells. Human cells may include, for example, T cells, Natural Killer (NK) cells, NK T cells, alpha-beta T cells, gamma-delta T-cells, cytotoxic T lymphocytes (CTL), regulatory T cells, B cells, human embryonic stem cells, tumor-infiltrating lymphocytes (TIL) or a pluripotent stem cell from which lymphoid cells may be differentiated (e.g., an induced pluripotent stem cell (iPSC)). In some embodiments, the systems can be used to modify pluripotent stem cells prior to their differentiation into multiple cell types. For example, a lymphoid cell precursor may be modified prior to differentiation into lymphoid cell types such as regulatory T cells, effector T cells, natural killer cells, etc. Systems of the present disclosure comprising more than one Bxb1 recombinase variant or orthologous recombinase variant, in particular, can be used to prepare cells with multiple integrated, excised, or inverted genes at once, including pluripotent cells. In some embodiments, systems containing more than one of the provided recombinase variants may be used to prepare, e.g., allogeneic T cells.

For agricultural applications, any method for introduction of proteins or nucleic acid molecules to a plant cell is also contemplated, such as Agrobacterium tumefaciens-mediated T-DNA delivery.

### III. Therapeutic Applications

The present disclosure provides methods of integrating, excising, and inverting a gene or sequence of DNA in cellular DNA, comprising delivering a Bxb1 recombinase variant or orthologous recombinase variant system described herein to a cell (e.g., from a patient). The cell may be within a patient (in vivo treatment), or a method as described herein may be performed on a cell removed from a patient and then the edited cell delivered to the patient (ex vivo treatment). In some embodiments, the cells are further manipulated ex vivo prior to use as a treatment. The term “treating” encompasses alleviation of symptoms, prevention of onset of symptoms, slowing of disease progression, improvement of quality of life, and increased survival. In some embodiments, a patient treated by the methods described herein is a mammal, e.g., a human.

In some embodiments, the methods of the present disclosure are used to insert or excise a gene or regulatory sequence associated with a disease towards restoring normal gene expression or activity. In some embodiments, the methods of the present disclosure may target a particular allele of a gene, e.g., a wild-type or mutated allele. In certain embodiments, the allele may be associated with cancer.

In some embodiments, the patient has cancer. In certain embodiments, the cell from the patient is further modified before or after gene editing to provide resistance to a chemotherapeutic agent. The patient may then be treated with the chemotherapeutic agent, which in some embodiments may result in greater survival of edited over unedited cells.

In some embodiments, the patient has an autoimmune disorder.

In some embodiments, the patient has an autosomal dominant disease, such as autosomal dominant polycystic kidney disease.

In some embodiments, the patient has a neuro-developmental disorder.

In some embodiments, the patient has a mitochondrial disorder.

In some embodiments, the patient has sickle cell disease, hemophilia (e.g., hemophilia A, B, or C), cystic fibrosis, phenylketonuria, Tay-Sachs, prion disease, color blindness, a lysosomal storage disease (e.g., Fabry disease), Friedreich's ataxia, prostate cancer, beta thalassemia, Huntington's disease, renal transplant, inflammatory bowel disease, multiple sclerosis, amyotrophic lateral sclerosis, or frontotemporal dementia.

The present disclosure further provides a pharmaceutical composition comprising elements of the gene editing system described herein, such as a Bxb1 recombinase variant or orthologous recombinase variant, or nucleotide sequences encoding said elements (e.g., in viral or non-viral vectors as described herein). The pharmaceutical composition may further comprise a pharmaceutically acceptable carrier such as water, saline (e.g., phosphate-buffered saline), dextrose, glycerol, sucrose, lactose, gelatin, dextran, albumin, or pectin. In addition, the composition may contain auxiliary substances, such as, wetting or emulsifying agents, pH-buffering agents, stabilizing agents, or other reagents that enhance the effectiveness of the pharmaceutical composition. The pharmaceutical composition may contain delivery vehicles such as liposomes, nanocapsules, microparticles, microspheres, lipid particles, and vesicles.

In some embodiments, the provided recombinase variants described herein may be used in a method of treatment described herein, may be for use in a treatment described herein, or may be used in the manufacture of a medicament for a treatment described herein.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of neurology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### EXAMPLES

### Example 1: attB and attP Plasmid Variants

The following are the wildtype Bxb1 recombinase consensus target sequences (the underlined sequence 5′-GGTTTGT is the ZD domain binding site, the underlined sequence 5′-ACNACNG is the RD domain binding site, and the center dinucleotide position is shown as XX in bold):

attP Consensus:
(SEQ ID NO: 81)
5′-GGTTTGTNNNNNNNACNACNGNNXXNNCNGTNGTNNNNNNNACAAA
CCCCAAACANNNNNNNTGNTGNCNNXXNNGNCANCANNNNNNNTGTTTG
G-5′
attB Consensus:
(SEQ ID NO: 82)
5′-GGTTTGTNNACNACNGNNXXNNCNGTNGTNNACAAACC
CCAAACANNTGNTGNCNNXXNNGNCANCANNTGTTTGG-5′

To simplify searching for Bxb1 recombinase variants with altered target DNA specificity, a more symmetric version of the Bxb1 attB target site was created (Table 4; see SEQ ID NO: 2 for the wildtype attB site). This facilitated the creation of symmetric mutations to both halves of the attB target site. As the wildtype attP site is already palindromic at the ZD and RD domain binding sites (see SEQ ID NO: 3), a palindromic version did not have to be created. As with the attB plasmid variants, the individual nucleotides of the binding domains were mutated to produce a construct with each nucleotide available (Table 5), as shown in bold for mutations at positions 9 and 10 below (outer and inner underlined sequences are bound by ZD and RD domains, respectively; center dinucleotides are italicized). “SEQ” in the tables refer to SEQ ID NOs.

SEQ ID NO

Mutations at position 10:

| | | |
|---|---|---|
| attB | GGTTTGTCGACGACGGCGGTCTCCGTCGTCAACAAACC | 80 |
| | GGTTTGTCGCCGACGGCGGTCTCCGTCGGCAACAAACC | 252 |
| | GGTTTGTCGGCGACGGCGGTCTCCGTCGCCAACAAACC | 253 |
| | GGTTTGTCGTCGACGGCGGTCTCCGTCGACAACAAACC | 254 |
| attP | GGTTTGTCTGGTCAACCACCGCGGTCTCAGTGGTGTACGGTACAAACC | 3 |
| | GGTTTGTCTGGTCACCCACCGCGGTCTCAGTGGGGTACGGTACAAACC | 255 |
| | GGTTTGTCTGGTCAGCCACCGCGGTCTCAGTGGCGTACGGTACAAACC | 256 |
| | GGTTTGTCTGGTCATCCACCGCGGTCTCAGTGGAGTACGGTACAAACC | 257 |

Mutations at position 9:

| | | |
|---|---|---|
| attB | GGTTTGTCGACGACGGCGGTCTCCGTCGTCAACAAACC | 80 |
| | GGTTTGTCGAAGACGGCGGTCTCCGTCTTCAACAAACC | 258 |
| | GGTTTGTCGAGGACGGCGGTCTCCGTCCTCAACAAACC | 259 |
| | GGTTTGTCGATGACGGCGGTCTCCGTCATCAACAAACC | 260 |
| attP | GGTTTGTCTGGTCAACCACCGCGGTCTCAGTGGTGTACGGTACAAACC | 3 |
| | GGTTTGTCTGGTCAAACACCGCGGTCTCAGTGTTGTACGGTACAAACC | 261 |
| | GGTTTGTCTGGTCAAGCACCGCGGTCTCAGTGCTGTACGGTACAAACC | 262 |
| | GGTTTGTCTGGTCAATCACCGCGGTCTCAGTGATGTACGGTACAAACC | 263 |

TABLE 4
Variant attB Sequences

| SEQ | Description | Sequence |
|---|---|---|
| 4 | Bxb1_AttB_P | ATGCTTCTCTGCATCCGTCTGCTAAATCCGCGTGATAGGGGAGGTTTGTC |
| | alindrome | GACGACGGCGGTCTCCGTCGTCAACAAACCCCGGCAGATAAAAGTACCCA |
| | | GAACCAGAGCCTACAATACCGGCCCTGGGAATATAAG |
| 5 | Bxb1_AttB_t | ATGCTTCTCTGCATCCGTCTGCTAAATCCGCGTGATAGGGGAtGTTTGTC |
| | GTTTGTCGACG | GACGACGGCGGTCTCCGTCGTCAACAAACaCCGGCAGATAAAAGTACCCA |
| | ACGGCGGT | GAACCAGAGCCTACAATACCGGCCCTGGGAATATAAG |
| 6 | Bxb1_AttB_c | ATGCTTCTCTGCATCCGTCTGCTAAATCCGCGTGATAGGGGACGTTTGTC |
| | GTTTGTCGACG | GACGACGGCGGTCTCCGTCGTCAACAAACgCCGGCAGATAAAAGTACCCA |
| | ACGGCGGT | GAACCAGAGCCTACAATACCGGCCCTGGGAATATAAG |
| 7 | Bxb1_AttB_a | ATGCTTCTCTGCATCCGTCTGCTAAATCCGCGTGATAGGGGAaGTTTGTC |
| | GTTTGTCGACG | GACGACGGCGGTCTCCGTCGTCAACAAACtCCGGCAGATAAAAGTACCCA |
| | ACGGCGGT | GAACCAGAGCCTACAATACCGGCCCTGGGAATATAAG |
| 8 | Bxb1_AttB_G | ATGCTTCTCTGCATCCGTCTGCTAAATCCGCGTGATAGGGGAGtTTTGTC |
| | tTTTGTCGACG | GACGACGGCGGTCTCCGTCGTCAACAAAaCCCGGCAGATAAAAGTACCCA |
| | ACGGCGGT | GAACCAGAGCCTACAATACCGGCCCTGGGAATATAAG |
| 9 | Bxb1_AttB | ATGCTTCTCTGCATCCGTCTGCTAAATCCGCGTGATAGGGGAGCTTTGTC |
| | CTTTGTCGACG | GACGACGGCGGTCTCCGTCGTCAACAAAgCCCGGCAGATAAAAGTACCCA |
| | ACGGCGGT | GAACCAGAGCCTACAATACCGGCCCTGGGAATATAAG |
| 10 | Bxb1_AttB_G | ATGCTTCTCTGCATCCGTCTGCTAAATCCGCGTGATAGGGGAGaTTTGTC |
| | aTTTGTCGACG | GACGACGGCGGTCTCCGTCGTCAACAAAtCCCGGCAGATAAAAGTACCCA |
| | ACGGCGGT | GAACCAGAGCCTACAATACCGGCCCTGGGAATATAAG |
| 11 | Bxb1_AttB_G | ATGCTTCTCTGCATCCGTCTGCTAAATCCGCGTGATAGGGGAGGgTTGTC |
| | GgTTGTCGACG | GACGACGGCGGTCTCCGTCGTCAACAAcCCCCGGCAGATAAAAGTACCCA |
| | ACGGCGGT | GAACCAGAGCCTACAATACCGGCCCTGGGAATATAAG |
| 12 | Bxb1_AttB_G | ATGCTTCTCTGCATCCGTCTGCTAAATCCGCGTGATAGGGGAGGaTTGTC |
| | GaTTGTCGACG | GACGACGGCGGTCTCCGTCGTCAACAAtCCCCGGCAGATAAAAGTACCCA |
| | ACGGCGGT | GAACCAGAGCCTACAATACCGGCCCTGGGAATATAAG |
| 13 | Bxb1_AttB_G | ATGCTTCTCTGCATCCGTCTGCTAAATCCGCGTGATAGGGGAGGCTTGTC |
| | GcTTGTCGACG | GACGACGGCGGTCTCCGTCGTCAACAAgCCCCGGCAGATAAAAGTACCCA |
| | ACGGCGGT | GAACCAGAGCCTACAATACCGGCCCTGGGAATATAAG |
| 14 | Bxb1_AttB_G | ATGCTTCTCTGCATCCGTCTGCTAAATCCGCGTGATAGGGGAGGTgTGTC |
| | GTgTGTCGACG | GACGACGGCGGTCTCCGTCGTCAACACACCCCGGCAGATAAAAGTACCCA |
| | ACGGCGGT | GAACCAGAGCCTACAATACCGGCCCTGGGAATATAAG |
| 15 | Bxb1_AttB | ATGCTTCTCTGCATCCGTCTGCTAAATCCGCGTGATAGGGGAGGTaTGTC |
| | GTaTGTCGACG | GACGACGGCGGTCTCCGTCGTCAACAtACCCCGGCAGATAAAAGTACCCA |
| | ACGGCGGT | GAACCAGAGCCTACAATACCGGCCCTGGGAATATAAG |
| 16 | Bxb1_AttB_G | ATGCTTCTCTGCATCCGTCTGCTAAATCCGCGTGATAGGGGAGGTCTGTC |
| | GTCTGTCGACG | GACGACGGCGGTCTCCGTCGTCAACAgACCCCGGCAGATAAAAGTACCCA |
| | ACGGCGGT | GAACCAGAGCCTACAATACCGGCCCTGGGAATATAAG |
| 17 | Bxb1_AttB_G | ATGCTTCTCTGCATCCGTCTGCTAAATCCGCGTGATAGGGGAGGTTgGTC |
| | GTTgGTCGACG | GACGACGGCGGTCTCCGTCGTCAACCAACCCCGGCAGATAAAAGTACCCA |
| | ACGGCGGT | GAACCAGAGCCTACAATACCGGCCCTGGGAATATAAG |
| 18 | Bxb1_AttB_G | ATGCTTCTCTGCATCCGTCTGCTAAATCCGCGTGATAGGGGAGGTTaGTC |
| | GTTaGTCGACG | GACGACGGCGGTCTCCGTCGTCAACtAACCCCGGCAGATAAAAGTACCCA |
| | ACGGCGGT | GAACCAGAGCCTACAATACCGGCCCTGGGAATATAAG |
| 19 | Bxb1_AttB_G | ATGCTTCTCTGCATCCGTCTGCTAAATCCGCGTGATAGGGGAGGTTCGTC |
| | GTTCGTCGACG | GACGACGGCGGTCTCCGTCGTCAACgAACCCCGGCAGATAAAAGTACCCA |
| | ACGGCGGT | GAACCAGAGCCTACAATACCGGCCCTGGGAATATAAG |
| 20 | Bxb1_AttB_G | ATGCTTCTCTGCATCCGTCTGCTAAATCCGCGTGATAGGGGAGGTTTtTC |
| | GTTTtTCGACG | GACGACGGCGGTCTCCGTCGTCAAaAAACCCCGGCAGATAAAAGTACCCA |
| | ACGGCGGT | GAACCAGAGCCTACAATACCGGCCCTGGGAATATAAG |
| 21 | Bxb1_AttB_G | ATGCTTCTCTGCATCCGTCTGCTAAATCCGCGTGATAGGGGAGGTTTCTC |
| | GTTTcTCGACG | GACGACGGCGGTCTCCGTCGTCAAgAAACCCCGGCAGATAAAAGTACCCA |
| | ACGGCGGT | GAACCAGAGCCTACAATACCGGCCCTGGGAATATAAG |
| 22 | Bxb1_AttB_G | ATGCTTCTCTGCATCCGTCTGCTAAATCCGCGTGATAGGGGAGGTTTaTC |
| | GTTTaTCGACG | GACGACGGCGGTCTCCGTCGTCAAtAAACCCCGGCAGATAAAAGTACCCA |
| | ACGGCGGT | GAACCAGAGCCTACAATACCGGCCCTGGGAATATAAG |
| 23 | Bxb1_AttB_G | ATGCTTCTCTGCATCCGTCTGCTAAATCCGCGTGATAGGGGAGGTTTGgC |
| | GTTTGgCGACG | GACGACGGCGGTCTCCGTCGTCACCAAACCCCGGCAGATAAAAGTACCCA |
| | ACGGCGGT | GAACCAGAGCCTACAATACCGGCCCTGGGAATATAAG |
| 24 | Bxb1_AttB_G | ATGCTTCTCTGCATCCGTCTGCTAAATCCGCGTGATAGGGGAGGTTTGaC |
| | GTTTGaCGACG | GACGACGGCGGTCTCCGTCGTCAtCAAACCCCGGCAGATAAAAGTACCCA |
| | ACGGCGGT | GAACCAGAGCCTACAATACCGGCCCTGGGAATATAAG |
| 25 | Bxb1_AttB_G | ATGCTTCTCTGCATCCGTCTGCTAAATCCGCGTGATAGGGGAGGTTTGCC |
| | GTTTGcCGACG | GACGACGGCGGTCTCCGTCGTCAgCAAACCCCGGCAGATAAAAGTACCCA |
| | ACGGCGGT | GAACCAGAGCCTACAATACCGGCCCTGGGAATATAAG |
| 26 | Bxb1_AttB | ATGCTTCTCTGCATCCGTCTGCTAAATCCGCGTGATAGGGGAGGTTTGTC |
| | GTTTGTCGtCG | GtCGACGGCGGTCTCCGTCGaCAACAAACCCCGGCAGATAAAAGTACCCA |
| | ACGGCGGT | GAACCAGAGCCTACAATACCGGCCCTGGGAATATAAG |
| 27 | Bxb1_AttB_G | ATGCTTCTCTGCATCCGTCTGCTAAATCCGCGTGATAGGGGAGGTTTGTC |
| | GTTTGTCGgCG | GgCGACGGCGGTCTCCGTCGcCAACAAACCCCGGCAGATAAAAGTACCCA |
| | ACGGCGGT | GAACCAGAGCCTACAATACCGGCCCTGGGAATATAAG |
| 28 | Bxb1_AttB_G | ATGCTTCTCTGCATCCGTCTGCTAAATCCGCGTGATAGGGGAGGTTTGTC |
| | GTTTGTCGCCG | GcCGACGGCGGTCTCCGTCGgCAACAAACCCCGGCAGATAAAAGTACCCA |
| | ACGGCGGT | GAACCAGAGCCTACAATACCGGCCCTGGGAATATAAG |
| 29 | Bxb1_AttB_G | ATGCTTCTCTGCATCCGTCTGCTAAATCCGCGTGATAGGGGAGGTTTGTC |
| | GTTTGTCGAgG | GAgGACGGCGGTCTCCGTCCTCAACAAACCCCGGCAGATAAAAGTACCCA |
| | ACGGCGGT | GAACCAGAGCCTACAATACCGGCCCTGGGAATATAAG |
| 30 | Bxb1_AttB | ATGCTTCTCTGCATCCGTCTGCTAAATCCGCGTGATAGGGGAGGTTTGTC |
| | GTTTGTCGAtG | GAtGACGGCGGTCTCCGTCaTCAACAAACCCCGGCAGATAAAAGTACCCA |
| | ACGGCGGT | GAACCAGAGCCTACAATACCGGCCCTGGGAATATAAG |
| 31 | Bxb1_AttB_ | ATGCTTCTCTGCATCCGTCTGCTAAATCCGCGTGATAGGGGAGGTTTGTC |
| | GTTTGTCGAaG | GAaGACGGCGGTCTCCGTCtTCAACAAACCCCGGCAGATAAAAGTACCCA |
| | ACGGCGGT | GAACCAGAGCCTACAATACCGGCCCTGGGAATATAAG |
| 32 | Bxb1_AttB_G | ATGCTTCTCTGCATCCGTCTGCTAAATCCGCGTGATAGGGGAGGTTTGTC |
| | GTTTGTCGACt | GACtACGGCGGTCTCCGTaGTCAACAAACCCCGGCAGATAAAAGTACCCA |
| | ACGGCGGT | GAACCAGAGCCTACAATACCGGCCCTGGGAATATAAG |
| 33 | Bxb1_AttB_G | ATGCTTCTCTGCATCCGTCTGCTAAATCCGCGTGATAGGGGAGGTTTGTC |
| | GTTTGTCGACC | GACCACGGCGGTCTCCGTgGTCAACAAACCCCGGCAGATAAAAGTACCCA |
| | ACGGCGGT | GAACCAGAGCCTACAATACCGGCCCTGGGAATATAAG |
| 34 | Bxb1_AttB_G | ATGCTTCTCTGCATCCGTCTGCTAAATCCGCGTGATAGGGGAGGTTTGTC |
| | GTTTGTCGACa | GACaACGGCGGTCTCCGTtGTCAACAAACCCCGGCAGATAAAAGTACCCA |
| | ACGGCGGT | GAACCAGAGCCTACAATACCGGCCCTGGGAATATAAG |
| 35 | Bxb1_AttB_G | ATGCTTCTCTGCATCCGTCTGCTAAATCCGCGTGATAGGGGAGGTTTGTC |
| | GTTTGTCGACG | GACGtCGGCGGTCTCCGaCGTCAACAAACCCCGGCAGATAAAAGTACCCA |
| | tCGGCGGT | GAACCAGAGCCTACAATACCGGCCCTGGGAATATAAG |
| 36 | Bxb1_AttB_G | ATGCTTCTCTGCATCCGTCTGCTAAATCCGCGTGATAGGGGAGGTTTGTC |
| | GTTTGTCGACG | GACGgCGGCGGTCTCCGCCGTCAACAAACCCCGGCAGATAAAAGTACCCA |
| | gCGGCGGT | GAACCAGAGCCTACAATACCGGCCCTGGGAATATAAG |
| 37 | Bxb1_AttB_G | ATGCTTCTCTGCATCCGTCTGCTAAATCCGCGTGATAGGGGAGGTTTGTC |
| | GTTTGTCGACG | GACGcCGGCGGTCTCCGgCGTCAACAAACCCCGGCAGATAAAAGTACCCA |
| | CCGGCGGT | GAACCAGAGCCTACAATACCGGCCCTGGGAATATAAG |
| 38 | Bxb1_AttB_G | ATGCTTCTCTGCATCCGTCTGCTAAATCCGCGTGATAGGGGAGGTTTGTC |
| | GTTTGTCGACG | GACGAgGGCGGTCTCCCTCGTCAACAAACCCCGGCAGATAAAAGTACCCA |
| | AgGGCGGT | GAACCAGAGCCTACAATACCGGCCCTGGGAATATAAG |
| 39 | Bxb1_AttB_G | ATGCTTCTCTGCATCCGTCTGCTAAATCCGCGTGATAGGGGAGGTTTGTC |
| | GTTTGTCGACG | GACGAtGGCGGTCTCCaTCGTCAACAAACCCCGGCAGATAAAAGTACCCA |
| | AtGGCGGT | GAACCAGAGCCTACAATACCGGCCCTGGGAATATAAG |
| 40 | Bxb1_AttB_G | ATGCTTCTCTGCATCCGTCTGCTAAATCCGCGTGATAGGGGAGGTTTGTC |
| | GTTTGTCGACG | GACGAaGGCGGTCTCCtTCGTCAACAAACCCCGGCAGATAAAAGTACCCA |
| | AaGGCGGT | GAACCAGAGCCTACAATACCGGCCCTGGGAATATAAG |

TABLE 5
Variant attP Sequences

| SEQ | Description | Sequence |
|---|---|---|
| 41 | AttP_TCCAC | GGTTTGTCTGGTCATCCACCGCGGTCTCAGTGGAGTACGGTACAAACC |
| | | CAATAAAAGTACCCAGAACCAGAGCCTACAATACCGGCCCTGGGAATA |
| | | TAAG |
| 42 | AttP_CCCAC | GGTTTGTCTGGTCACCCACCGCGGTCTCAGTGGGGTACGGTACAAACC |
| | | CAATAAAAGTACCCAGAACCAGAGCCTACAATACCGGCCCTGGGAATA |
| | | TAAG |
| 43 | AttP_GCCAG | GGTTTGTCTGGTCAGCCACCGCGGTCTCAGTGGCGTACGGTACAAACC |
| | | CAATAAAAGTACCCAGAACCAGAGCCTACAATACCGGCCCTGGGAATA |
| | | TAAG |
| 44 | AttP_AACAC | GGTTTGTCTGGTCAAACACCGCGGTCTCAGTGTTGTACGGTACAAACC |
| | | CAATAAAAGTACCCAGAACCAGAGCCTACAATACCGGCCCTGGGAATA |
| | | TAAG |
| 45 | AttP_ATCAC | GGTTTGTCTGGTCAATCACCGCGGTCTCAGTGATGTACGGTACAAACC |
| | | CAATAAAAGTACCCAGAACCAGAGCCTACAATACCGGCCCTGGGAATA |
| | | TAAG |
| 46 | AttP_AGCAC | GGTTTGTCTGGTCAAGCACCGCGGTCTCAGTGCTGTACGGTACAAACC |
| | | CAATAAAAGTACCCAGAACCAGAGCCTACAATACCGGCCCTGGGAATA |
| | | TAAG |
| 47 | AttP_ACTAC | GGTTTGTCTGGTCAACTACCGCGGTCTCAGTAGTGTACGGTACAAACC |
| | | CAATAAAAGTACCCAGAACCAGAGCCTACAATACCGGCCCTGGGAATA |
| | | TAAG |
| 48 | AttP_ACAAC | GGTTTGTCTGGTCAACAACCGCGGTCTCAGTTGTGTACGGTACAAACC |
| | | CAATAAAAGTACCCAGAACCAGAGCCTACAATACCGGCCCTGGGAATA |
| | | TAAG |
| 49 | AttP_ACGAC | GGTTTGTCTGGTCAACGACCGCGGTCTCAGTCGTGTACGGTACAAACC |
| | | CAATAAAAGTACCCAGAACCAGAGCCTACAATACCGGCCCTGGGAATA |
| | | TAAG |
| 50 | AttP_ACCTC | GGTTTGTCTGGTCAACCTCCGCGGTCTCAGAGGTGTACGGTACAAACC |
| | | CAATAAAAGTACCCAGAACCAGAGCCTACAATACCGGCCCTGGGAATA |
| | | TAAG |
| 51 | AttP_ACCCC | GGTTTGTCTGGTCAACCCCCGCGGTCTCAGGGGTGTACGGTACAAACC |
| | | CAATAAAAGTACCCAGAACCAGAGCCTACAATACCGGCCCTGGGAATA |
| | | TAAG |
| 52 | AttP_ACCGC | GGTTTGTCTGGTCAACCGCCGCGGTCTCAGCGGTGTACGGTACAAACC |
| | | CAATAAAAGTACCCAGAACCAGAGCCTACAATACCGGCCCTGGGAATA |
| | | TAAG |
| 53 | AttP_ACCAG | GGTTTGTCTGGTCAACCAGCGCGGTCTCACTGGTGTACGGTACAAACC |
| | | CAATAAAAGTACCCAGAACCAGAGCCTACAATACCGGCCCTGGGAATA |
| | | TAAG |
| 54 | AttP_ACCAT | GGTTTGTCTGGTCAACCATCGCGGTCTCAATGGTGTACGGTACAAACC |
| | | CAATAAAAGTACCCAGAACCAGAGCCTACAATACCGGCCCTGGGAATA |
| | | TAAG |
| 55 | AttP_ACCAA | GGTTTGTCTGGTCAACCAACGCGGTCTCATTGGTGTACGGTACAAACC |
| | | CAATAAAAGTACCCAGAACCAGAGCCTACAATACCGGCCCTGGGAATA |
| | | TAAG |
| 56 | AttP_CGTTTGT | CGTTTGTCTGGTCAACCACCGCGGTCTCAGTGGTGTACGGTACAAACG |
| | | CAATAAAAGTACCCAGAACCAGAGCCTACAATACCGGCCCTGGGAATA |
| | | TAAG |
| 57 | AttP_TGTTTGT | TGTTTGTCTGGTCAACCACCGCGGTCTCAGTGGTGTACGGTACAAACA |
| | | CAATAAAAGTACCCAGAACCAGAGCCTACAATACCGGCCCTGGGAATA |
| | | TAAG |
| 58 | AttP_AGTTTGT | AGTTTGTCTGGTCAACCACCGCGGTCTCAGTGGTGTACGGTACAAACT |
| | | CAATAAAAGTACCCAGAACCAGAGCCTACAATACCGGCCCTGGGAATA |
| | | TAAG |
| 59 | AttP_GCTTTGT | GCTTTGTCTGGTCAACCACCGCGGTCTCAGTGGTGTACGGTACAAAGC |
| | | CAATAAAAGTACCCAGAACCAGAGCCTACAATACCGGCCCTGGGAATA |
| | | TAAG |
| 60 | AttP_GTTTTGT | GTTTTGTCTGGTCAACCACCGCGGTCTCAGTGGTGTACGGTACAAAAC |
| | | CAATAAAAGTACCCAGAACCAGAGCCTACAATACCGGCCCTGGGAATA |
| | | TAAG |
| 61 | AttP_GATTTGT | GATTTGTCTGGTCAACCACCGCGGTCTCAGTGGTGTACGGTACAAATC |
| | | CAATAAAAGTACCCAGAACCAGAGCCTACAATACCGGCCCTGGGAATA |
| | | TAAG |
| 62 | AttP_GGATTGT | GGATTGTCTGGTCAACCACCGCGGTCTCAGTGGTGTACGGTACAATCC |
| | | CAATAAAAGTACCCAGAACCAGAGCCTACAATACCGGCCCTGGGAATA |
| | | TAAG |
| 63 | AttP_GGCTTGT | GGCTTGTCTGGTCAACCACCGCGGTCTCAGTGGTGTACGGTACAAGCC |
| | | CAATAAAAGTACCCAGAACCAGAGCCTACAATACCGGCCCTGGGAATA |
| | | TAAG |
| 64 | AttP_GGGTTGT | GGGTTGTCTGGTCAACCACCGCGGTCTCAGTGGTGTACGGTACAACCC |
| | | CAATAAAAGTACCCAGAACCAGAGCCTACAATACCGGCCCTGGGAATA |
| | | TAAG |
| 65 | AttP_GGTCTGT | GGTCTGTCTGGTCAACCACCGCGGTCTCAGTGGTGTACGGTACAGACC |
| | | CAATAAAAGTACCCAGAACCAGAGCCTACAATACCGGCCCTGGGAATA |
| | | TAAG |
| 66 | AttP_GGTGTGT | GGTGTGTCTGGTCAACCACCGCGGTCTCAGTGGTGTACGGTACACACC |
| | | CAATAAAAGTACCCAGAACCAGAGCCTACAATACCGGCCCTGGGAATA |
| | | TAAG |
| 67 | AttP_GGTATGT | GGTATGTCTGGTCAACCACCGCGGTCTCAGTGGTGTACGGTACATACC |
| | | CAATAAAAGTACCCAGAACCAGAGCCTACAATACCGGCCCTGGGAATA |
| | | TAAG |
| 68 | AttP_GGTTAGT | GGTTAGTCTGGTCAACCACCGCGGTCTCAGTGGTGTACGGTACTAACC |
| | | CAATAAAAGTACCCAGAACCAGAGCCTACAATACCGGCCCTGGGAATA |
| | | TAAG |
| 69 | AttP_GGTTCGT | GGTTCGTCTGGTCAACCACCGCGGTCTCAGTGGTGTACGGTACGAACC |
| | | CAATAAAAGTACCCAGAACCAGAGCCTACAATACCGGCCCTGGGAATA |
| | | TAAG |
| 70 | AttP_GGTTGGT | GGTTGGTCTGGTCAACCACCGCGGTCTCAGTGGTGTACGGTACCAACC |
| | | CAATAAAAGTACCCAGAACCAGAGCCTACAATACCGGCCCTGGGAATA |
| | | TAAG |
| 71 | AttP_GGTTTTT | GGTTTTTCTGGTCAACCACCGCGGTCTCAGTGGTGTACGGTAAAAACC |
| | | CAATAAAAGTACCCAGAACCAGAGCCTACAATACCGGCCCTGGGAATA |
| | | TAAG |
| 72 | AttP_GGTTTAT | GGTTTATCTGGTCAACCACCGCGGTCTCAGTGGTGTACGGTATAAACC |
| | | CAATAAAAGTACCCAGAACCAGAGCCTACAATACCGGCCCTGGGAATA |
| | | TAAG |
| 73 | AttP_GGTTTCT | GGTTTCTCTGGTCAACCACCGCGGTCTCAGTGGTGTACGGTAGAAACC |
| | | CAATAAAAGTACCCAGAACCAGAGCCTACAATACCGGCCCTGGGAATA |
| | | TAAG |
| 74 | AttP_GGTTTGA | GGTTTGACTGGTCAACCACCGCGGTCTCAGTGGTGTACGGTTCAAACC |
| | | CAATAAAAGTACCCAGAACCAGAGCCTACAATACCGGCCCTGGGAATA |
| | | TAAG |
| 75 | AttP_GGTTTGC | GGTTTGCCTGGTCAACCACCGCGGTCTCAGTGGTGTACGGTGCAAACC |
| | | CAATAAAAGTACCCAGAACCAGAGCCTACAATACCGGCCCTGGGAATA |
| | | TAAG |
| 76 | AttP_GGTTTGG | GGTTTGGCTGGTCAACCACCGCGGTCTCAGTGGTGTACGGTCCAAACC |
| | | CAATAAAAGTACCCAGAACCAGAGCCTACAATACCGGCCCTGGGAATA |
| | | TAAG |

### Example 2: Initial Serine Scan Screen

Individual constructs with serine scan mutations were generated from a pVax vector that expresses Bxb1 recombinase driven by a CMV promoter. The Bxb1 recombinase construct included the amino acid sequence GSGSGSHHHHHHGSGPKKKRKV (SEQ ID NO: 249) at the C terminal end. Individual constructs matching SEQ ID NOs: 2-76 were generated in a non-expression plasmid. Sets of constructs were pooled together such that all four bases at a specific site were in equal concentration (e.g., plasmids with attP SEQ ID NOs: 3, 41, 42, and 43 were pooled together and SEQ ID NOs: 3, 44, 45, and 46 were pooled together; plasmids with attB SEQ ID NOs: 4, 5, 6, and 7 were pooled together and SEQ ID NOs: 4, 8, 9, and 10 were pooled together).

K562 cells were transfected following the manufacturer's protocol. 2E5 cells were transfected with 200 ng of Bxb1 expression plasmid, 20 ng total of attB plasmid, and 1600 ng total of attP plasmid. The attB plasmids had 5 ng each of pooled nucleotide mutants at a specific site. For example, when testing base 24 (FIG. 2), SEQ ID NOs: 4, 5, 6, and 7 were pooled, such that 5 ng of each were delivered in 20 ng of total attB plasmid. In that same well, SEQ ID NOs: 3, 56, 57, and 58 were pooled, such that 400 ng of each were delivered in 1600 ng total of attP plasmid.

Individual expression plasmids with the mutations M244S, L245S, G246S, Y247S, A248S, L250S, N251S, G252S, K253S, T254S, V255S, R256S, D257S, D258S, D259S, G260S, A262S, K284S, T285S, S286A, R287S, A288S, K289S, P290S, A291S, V292S, S293A, T294S, P295S, S296A, L297S, L298S, L299S, R300S, V301S, A311S, Y312S, K313S, F314S, A315S, G316S, G317S, G318S, R319S, K320S, H321S, P322S, R323S, Y324S, R325S, C326S, R327S, S328A, M329S, G330S, F331S, P332S, K333S, H334S, C335S, E445S, Q446S, D447S, A449S, A450S, K451S, Y452S, T453S, W454S, L455S, R456S, M458S, N459S, and V460S were transfected across libraries for all seven nucleotides thought to be recognized by the ZD domains.

Individual expression plasmids with the mutations I137S, K138S, E139S, R140S, N141S, R142S, S143A, A144S, A145S, H146S, F147S, N148S, 1149S, R150S, A151S, G152S, K153S, Y154S, R155S, G156S, S157A, L158S, P159S, P160S, H195S, E196S, P197S, L198S, H199S, L200S, V201S, E229S, W230S, S231A, A232S, T233S, A234S, L235S, K236S, R237S, S238A, M239S, M244S, L245S, G246S, Y247S, A248S, T249S, L250S, N251S, G252S, K253S, T254S, V255S, R256S, D257S, D258S, D259S, G260S, A262S, K284S, T285S, S286A, R287S, A288S, K289S, P290S, A291S, V292S, S293A, T294S, P295S, S296A, L297S, L298S, L299S, R300S, V301S, A311S, Y312S, K313S, F314S, A315S, G316S, G317S, G318S, R319S, K320S, H321S, P322S, R323S, Y324S, R325S, C326S, R327S, S328A, M329S, G330S, F331S, P332S, K333S, H334S, and C335S were transfected across nucleotide mutant libraries spanning the five bases thought to be recognized by the RD domain (see FIG. 2).

Transfected cells were harvested after three days of incubation at 37° C. using QuickExtract™ by Lucigen (Cat No: QE09050) following the manufacturer's protocol. A polymerase chain reaction (PCR) was performed on the extracted DNA using Invitrogen's Accuprimer™ Taq DNA Polymerase, high fidelity (cat no: 12346094), as shown in Table 6 below.

TABLE 6
NGS PCR1 Setup

| Reagent | 1x (μl) | MM (μl) |
|---|---|---|
| 10x AccuPrime ™ Buffer II | 2.5 | 275 |
| 10 μM fw oligo | 1 | 110 |
| 10 μM rev oligo | 1 | 110 |
| AccuPrime ™ Taq (HiFi) | 0.2 | 22 |
| H₂O | 17.3 | 1903 |
| gDNA | 3 | |
| Total | 25 | 2420 |

| | |
|---|---|
| Rxns: 110 | Aliquot 22.0 μl of MM |
| | Add 3.0 μl of gDNA |

The PCR was performed using the primers shown in Table 7 below, where P1 and P2 refer to primers 1 and 2, respectively.

TABLE 7
NGS PCR1 primers

| | SEQ | |
|---|---|---|
| | ID NO | |
| P1 | 250 | ACACGACGCTCTTCCGATCTNNNNATGCTTCTCTGCATCCG |
| | | TCT |
| P2 | 251 | GACGTGTGCTCTTCCGATCTAGGGAATAGATGACATCAAAG |
| | | AGAA |

The PCR was performed using the temperature cycling protocol in Table 8 below. The format of the time is minutes: seconds.

TABLE 8
NGS PCR1 Program

| | |
|---|---|
| Temperature | Time |

| | | | |
|---|---|---|---|
| 95° | C. | 5:00 | |
| 95° | C. | 0:30 | 30x |
| 55° | C. | 0:30 | |
| 68° | C. | 0:40 | |
| 68° | C. | 10:00 | |
| 4° | C. | ∞ | |

The product of next generation sequencing (NGS) PCR1 was used as the template for NGS PCR2, as shown in Table 9 below.

TABLE 9
NGS PCR2 Setup

| Reagent | 1x (μl) | MM (μl) |
|---|---|---|
| 2x Phusion ™ HiFi PCR MM w/HF buffer | 10 | 1100 |
| 10 μM fw oligo (N5xx) | 1 | 110 |
| 10 μM rev oligo (Index) | 1 | |
| H₂O | 6.5 | 715 |
| gDNA | 1.5 | |
| Total | 20 | 1925 |
| Rxns: 110 | Aliquot 17.5 μl of MM | |
| | Add 1 μl of Index oligo | |
| | Add 1.5 μl of PCR #1 | |

The PCR was performed using the temperature cycling protocol in Table 10 below. The format of the time is minutes: seconds.

TABLE 10
NGS PCR2 Program

| | |
|---|---|
| Temperature | Time |

| | | | |
|---|---|---|---|
| 98° | C. | 0:30 | |
| 98° | C. | 0:10 | 12x |
| 60° | C. | 0:30 | |
| 72° | C. | 0:40 | |
| 72° | C. | 10:00 | |
| 4° | C. | ∞ | |

The primers used are universal primers that add on the required DNA sequences for Illumina Sequencing machines. NGS PCR2 products were purified and sequenced on Illumina Sequencing machines.

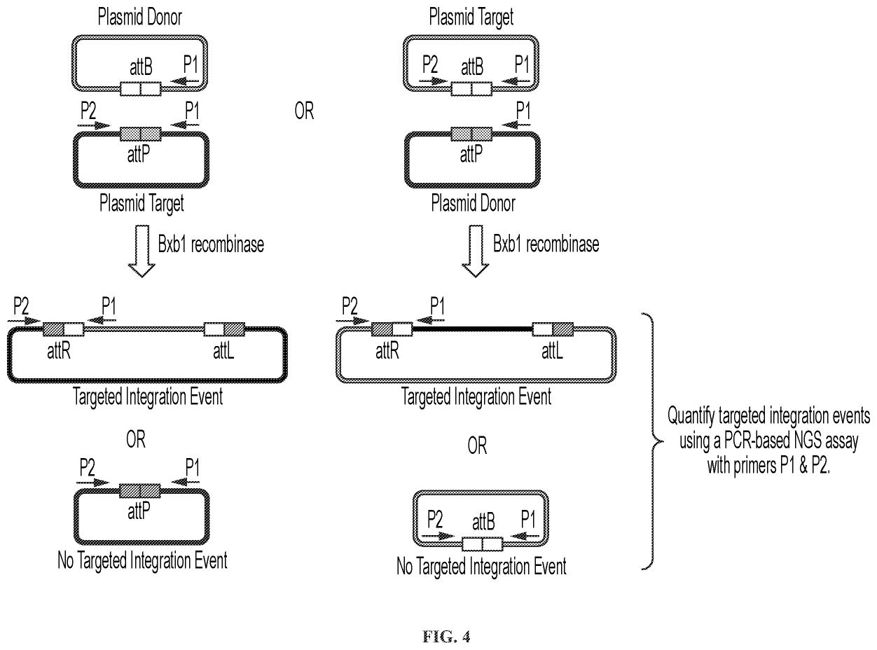

Sequences were analyzed for changes in specificity at the nucleotide sites that were pooled together. For example, for the samples tested with nucleotide 21, integration events that differed from the percentages seen by wild-type Bxb1 recombinase were examined and documented (FIG. 4).

### Example 3: Saturation Mutagenesis of Identified Residues

Saturation mutagenesis was performed at amino acids that showed a different integration profile during the serine scan of Example 2. These differences ranged from eliminating integration, relaxing specificity, increasing specificity, or changing specificity. Sites selected for saturation mutagenesis included F147, N148, 1149, Y154, R155, G156, L158, P197, L198, W230, S231, A232, T233, R237, D257, E309, Y312, F314, A315, G316, G318, R323, Y324, R325, C326, and C335. K562 transfections and NGS analysis were performed in the same manner described in Example 2 (FIG. 4).

A summary of the results of saturation mutagenesis in the RD domain of Bxb1 recombinase is shown in Table 11 below. “Specificity shift” refers to as the largest shift towards a non-WT nucleotide for a given mutant at a given position. For example, if WT Bxb1 recombinase has the following preference at position 9:4.5% A, 85.6% C, 1.0% G, and 8.9% T, and if the R237A mutant has the following preferences at position 9:23.6% A, 1.9% C, 17.1% G, and 57.4% T, then the largest shift towards a non-WT nucleotide is for T (57.4%-8.9%=48.5% change); R237A has a specificity shift at position 9 of 48.5%. The “average % specificity shift” is the average of the specificity shift for all mutants at a given amino acid position. “% TI” refers to the percent of successful targeted integration; it is calculated as the percent of sequence reads that had a sequence consistent with integration of the donor sequence, over the total sequence reads. In % TI for mutant a class (e.g., R237X) is the average for % TI for all members of that class (e.g., the average % TI for R237A, R237C, R237D, etc.) WT is the average of four replicates. In tables 11 and 12 below, “% TI” for a mutant class (e.g., R237X) is the average % TI for all members of that class (e.g., the average % TI for R237A, R237C, R237D, etc.), and WT is the average of four replicates.

TABLE 11
Bxb1 RD Domain Saturation Mutagenesis Results

| | | | Average % Specificity Shift | | | | |
|---|---|---|---|---|---|---|---|
| | Interesting Mutants | % TI | A | C | N | A | C |
| WT | NA | 77.4 | 0.0 | 0.0 | 0.0 | 0.0 | 0.0 |
| F147X | 3 | 33.1 | 8.4 | 10.7 | 4.6 | 9.4 | 18.9 |
| N148X | 2 | 61.8 | 2.5 | 2.7 | 1.1 | 2.7 | 4.5 |
| I149X | 0 | 8.4 | 0.0 | 0.0 | 0.0 | 0.0 | 0.0 |
| Y154X | 0 | 8.3 | 0.0 | 0.0 | 0.0 | 0.0 | 0.0 |
| R155X | 0 | 15.7 | 0.0 | 0.0 | 0.0 | 0.0 | 0.3 |
| G156X | 0 | 0.1 | 0.0 | 0.0 | 0.0 | 0.0 | 0.0 |
| L158X | 5 | 50.6 | 0.0 | 0.0 | 5.3 | 9.4 | 5.3 |
| P197X | 3 | 76.8 | 2.2 | 2.3 | 0.7 | 1.9 | 2.5 |
| L198X | 0 | 59.6 | 0.0 | 0.0 | 0.0 | 0.0 | 0.0 |
| W230X | 0 | 39.5 | 0.4 | 0.0 | 1.0 | 0.0 | 0.0 |
| S231X | 7 | 50.2 | 11.4 | 0.0 | 0.0 | 0.0 | 0.0 |
| A232X | 0 | 70.9 | 0.2 | 0.0 | 0.9 | 0.5 | 1.3 |
| T233X | 6 | 64.4 | 8.8 | 2.1 | 0.0 | 2.4 | 5.1 |
| R237X | 19 | 28.4 | 0.0 | 47.6 | 0.0 | 0.0 | 0.0 |
| D257X | 0 | 74.5 | 5.4 | 3.9 | 3.3 | 4.1 | 6.1 |

A summary of the results of saturation mutagenesis in the ZD domains of Bxb1 recombinase is shown in Table 12 below.

TABLE 12
Bxb1 ZD Domain Saturation Mutagenesis Results

| | Interesting | | Average % Specificity Shift | | | | | | |
|---|---|---|---|---|---|---|---|---|---|
| Substitutions | Mutants | % TI | G | G | T | T | T | G | T |
| WT | NA | 82.2 | 0.0 | 0.0 | 0.0 | 0.0 | 0.0 | 0.0 | 0.0 |
| E309X | 0 | 79.9 | 1.0 | 0.5 | 0.2 | 0.7 | 1.4 | 0.5 | 4.0 |
| Y312X | 0 | 25.5 | 0.0 | 0.0 | 0.1 | 0.8 | 0.6 | 0.8 | 8.3 |
| F314X | 15 | 35.7 | 1.4 | 1.2 | 18.5 | 59.8 | 7.5 | 13.8 | 8.7 |
| A315X | 18 | 82.6 | 4.6 | 5.5 | 8.3 | 6.7 | 1.1 | 2.8 | 5.1 |
| G316X | 4 | 67.0 | 5.0 | 25.8 | 11.8 | 24.9 | 0.1 | 0.2 | 0.0 |
| G318X | 2 | 79.1 | 7.7 | 2.4 | 3.9 | 4.7 | 2.5 | 0.6 | 1.2 |
| R323X | 2 | 8.2 | 4.3 | 4.5 | 5.2 | 3.6 | 0.2 | 0.6 | 0.0 |
| Y324X | 0 | 41.0 | 2.5 | 0.0 | 0.0 | 0.0 | 0.0 | 0.0 | 0.0 |
| R325X | 9 | 6.5 | 0.0 | 0.0 | 0.4 | 0.4 | 30.4 | 4.6 | 19.1 |
| C326X | 0 | 3.3 | 0.5 | 0.0 | 8.2 | 0.0 | 0.0 | 0.0 | 0.0 |
| C335X | 0 | 5.9 | 0.3 | 0.0 | 7.9 | 10.2 | 0.0 | 0.0 | 0.0 |

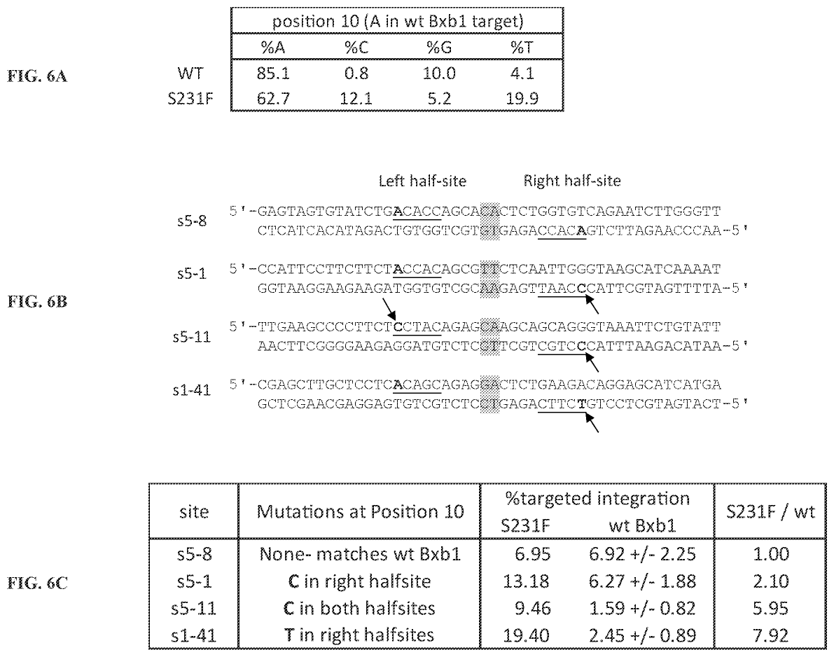

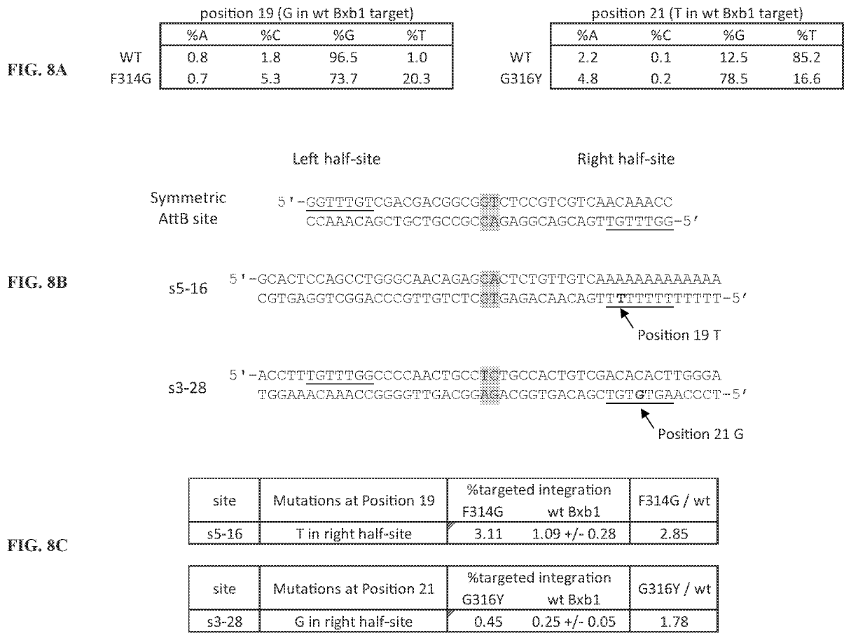

Detailed results for S231F at position 10 and WT Bxb1 from a side-by-side experiment are shown in FIG. 6A, and detailed results for F314G and G316Y at positions 19 and 21, respectively, are shown in FIG. 8A.

### Example 4: Bxb1-Mediated Targeted Integration at Endogenous Sites in Human Cells

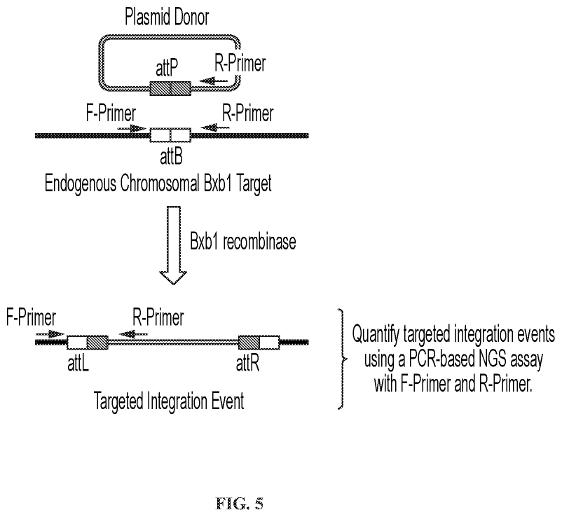

The performance of Bxb1 variants was characterized at a panel of endogenous Bxb1 sites where wild-type Bxb1 enables detectable integration activity (FIG. 5).

Bxb1 recombinase pseudo-attB target sequences in the human genome are shown in Table 13 below; the pseudo-attB site is flanked by five nucleotides on the 5′ and 3′ end, and the center dinucleotide position is shown in bold.

TABLE 13
Endogenous Bxb1 Pseudo-attB Target Sequences

| SEQ | Site | Bxb1 Target |
|---|---|---|
| 137 | s5-1 | CCATTCCTTCTTCTACCACAGCGTTCTCAATTGGGTAAGCA |
| | | TCAAAAT |
| 138 | s5-2 | TTTAGTTGCTCCTGACAGCAGTGGGAGCTATTGTCTAAGAG |
| | | ATACAAA |
| 139 | s5-4 | GGAGTTGAGTCCTGACTGCAGCACATGCAGGTGAGGAAGCA |
| | | GTAGAAG |
| 140 | s5-6 | TACTGGGGATTCCCATAACCGTGCACTCAGCTGCGGGAAGG |
| | | CAGGAGA |
| 141 | s5-8 | GAGTAGTGTATCTGACACCAGCACACTCTGGTGTCAGAATC |
| | | TTGGGTT |
| 142 | s5-10 | ATAGTGAAACTTACAACACAGAGTTTCCAGTTGTTCAATCT |
| | | CTCTTGT |
| 143 | s5-11 | TTGAAGCCCCTTCTCCTACAGAGCAAGCAGCAGGGTAAATT |
| | | CTGTATT |
| 144 | s5-14 | TGGTGCAATCTTGGCTCACTGCAACCTCTGCAGTTGAAGCA |
| | | ATTCTCC |
| 145 | s5-15 | ATTTGTGGTACTATACTACAGAGAATGCTGATGTTAAAGAG |
| | | CTAATTT |
| 146 | s5-16 | GCACTCCAGCCTGGGCAACAGAGCACTCTGTTGTCAAAAAA |
| | | AAAAAAA |
| 147 | s5-17 | ACACCTGGATCTCCACCCCTGTGTTTGCTGTTGTCTAAGTT |
| | | TTACATT |
| 148 | s5-18 | CCGTCATGTTCCTCACCACAGCTAACCCAGTCTTCAAAACT |
| | | CACTTAA |
| 149 | s5-19 | GGCCTCCTGCTTGCTCAGCTGAGACGGCAGAGGCTGAAGCT |
| | | GCACGGG |
| 150 | s5-21 | CTTCCTTCTTTTAAACCACTGATCATGCAGAAGTATAAGCT |
| | | CACCAGT |
| 151 | s5-24 | GTGGCAGAAATAGTACAACAGCTAACGCAGATGTTAAAAAT |
| | | CAAATGG |
| 152 | s1-1 | CAGTGTGCTTGTTGCCTACAGCCTCTGCAGAAGTTCACAAA |
| | | CTTGCAC |
| 153 | s1-5 | ATTAGGGGTTGGTGGCAATGGAGATCTCAGTGGTGAAAAAT |
| | | CACTTCT |
| 154 | s1-10 | ATGTGGTTTTGTTCACTGTAGAATCCTCAGTGGTTAGCACA |
| | | ATGTTAA |
| 155 | s1-11 | CATTTCTCTTGATGACTGCAGAGTATTCCATTGTTGACAAA |
| | | TCCATTT |
| 156 | s1-17 | GCTATGGGTTTTTAAACACTGACTTGCCTGGTGTGAGGATC |
| | | CATGCTT |
| 157 | s1-25 | GTGTTCTGATACTTGCAACTGAACACACTGTAGTCCACACA |
| | | CCACTAT |
| 158 | s1-28 | GGGGGTTGCTTCTCACCGTAGATGACCCTCTTGTCCACAAA |
| | | CCACAAC |
| 159 | s1-31 | CAGCCTGGCTCCCAACCTCCGTCACTGCCGTTGCAAACAAA |
| | | CCAAACC |
| 160 | s1-39 | TTTCAAAGATCCTGACCTCCGAAGAAGCTGGAGTTGAGATC |
| | | ATACTGT |
| 161 | s1-41 | CGAGCTTGCTCCTCACAGCAGAGGACTCTGAAGACAGGAGC |
| | | ATCATGA |
| 162 | s1-43 | CCTTGCAGATCTATACGGCTGACTACACAGTGGTGAGAACC |
| | | ATAGTCT |
| 163 | s3-28 | ACCTTTGTTTGGCCCCAACTGCCTCTGCCACTGTCGACACA |
| | | CTTGGGA |
| 164 | s3-41 | GATGCATGATTCTAGGCACTGGGAACACAGTGGTGATCAAA |
| | | CCAGTCA |

Individual donor constructs were generated by cloning the target site-specific sequences shown in Table 14 below into a standard plasmid DNA backbone. Each donor DNA sequence consists of a Bxb1 attP sequence with the target site matching the center dinucleotide, and a target site-specific primer binding site. The attP sequence and primer binding sites are flanking additional genomic sequences that were partially randomized to form a tag sequence to detect targeted integration events through next generation sequencing.

TABLE 14
Target Site-Specific Donor Sequences

| SEQ | Site | Donor |
|---|---|---|
| 165 | s5-1 | GGTTTGTCTGGTCAACCACCGCGTTCTCAGTGGTGTACGGT |
| | | ACAAACCTTCGTTTCAACTGACCAATTCACTCCAAATGTAA |
| | | CA |
| 166 | s5-2 | GGTTTGTCTGGTCAACCACCGCGGGCTCAGTGGTGTACGGT |
| | | ACAAACCAAGAGGAAAAGTATACCTGCAGCAAGTGATAT |
| 167 | s5-4 | GGTTTGTCTGGTCAACCACCGCGCACTCAGTGGTGTACGGT |
| | | ACAAACCATTGCTGCCGTCTGTCCCCTTCCCATCCCTTCTG |
| | | ACATGT |
| 168 | s5-6 | GGTTTGTCTGGTCAACCACCGCGCACTCAGTGGTGTACGGT |
| | | ACAAACCAGCCCGAACTTAGCTTCCCTCATCGCCTA |
| 169 | s5-8 | GGTTTGTCTGGTCAACCACCGCGCACTCAGTGGTGTACGGT |
| | | ACAAACCATATCGTTTGCTACTCAAACTGTGTGGTCCT |
| 170 | s5-10 | GGTTTGTCTGGTCAACCACCGCGTTCTCAGTGGTGTACGGT |
| | | ACAAACCTATCTTCCTTTTCTGTGGAAGGGAAAACTT |
| 171 | s5-11 | GGTTTGTCTGGTCAACCACCGCGCACTCAGTGGTGTACGGT |
| | | ACAAACCAAGCAGCTCGGAGCCTCTCTGTCACTTGCTCTTT |
| | | AGA |
| 172 | s5-14 | GGTTTGTCTGGTCAACCACCGCGACCTCAGTGGTGTACGGT |
| | | ACAAACCGCCTGTCACCTCCTGAGTACCTGGGATTACAGGC |
| | | GCACAACACTACTCT |
| 173 | s5-15 | GGTTTGTCTGGTCAACCACCGCGAACTCAGTGGTGTACGGT |
| | | ACAAACCTCAGGAGGTGTTTTTCCTTCCTTTTGAAATCAGA |
| | | TAGCAGGAAA |
| 174 | s5-16 | GGTTTGTCTGGTCAACCACCGCGCACTCAGTGGTGTACGGT |
| | | ACAAACCAAGAGCACAGTGATTATGATTATTGTTTCTGAGT |
| | | GATGGCA |
| 175 | s5-17 | GGTTTGTCTGGTCAACCACCGCGTTCTCAGTGGTGTACGGT |
| | | ACAAACCTAATGTCCGCACTTCCTAAGTTTTGGTTTCCAGA |
| | | GCCACTAGTAA |
| 176 | s5-18 | GGTTTGTCTGGTCAACCACCGCGAACTCAGTGGTGTACGGT |
| | | ACAAACCTCTATTCGCACTCAGGGAGGCCTTCCCTGATCTC |
| | | CAGGGCCAGATTAAGTA |
| 177 | s5-19 | GGTTTGTCTGGTCAACCACCGCGACCTCAGTGGTGTACGGT |
| | | ACAAACCCGGCCAGTCAGTCACTGTCAGGTGAGCACCCGCG |
| | | AGGGTCCCGGGGAGGTACGGGTGAC |
| 178 | s5-21 | GGTTTGTCTGGTCAACCACCGCGCACTCAGTGGTGTACGGT |
| | | ACAAACCACCTTCACGTCCTAACCAGACCATGAGCTCCAGG |
| | | GGTAAAATC |
| 179 | s5-24 | GGTTTGTCTGGTCAACCACCGCGAACTCAGTGGTGTACGGT |
| | | ACAAACCCATGTAAAACCAGCTTATTGCCAACAGAGTTATG |
| | | GA |
| 180 | s1-1 | GGTTTGTCTGGTCAACCACCGCGTCCTCAGTGGTGTACGGT |
| | | ACAAACCAGAATCAGGATAAGGCTCCTCTAGGTACATGTGG |
| | | CTAAGCTGC |
| 181 | s1-5 | GGTTTGTCTGGTCAACCACCGCGATCTCAGTGGTGTACGGT |
| | | ACAAACCGGTCCTCCATCAAGCGCTGAGGCACAGTGAATGT |
| | | ACTCAGAGGG |
| 182 | s1-10 | GGTTTGTCTGGTCAACCACCGCGTCCTCAGTGGTGTACGGT |
| | | ACAAACCACACAATAGCGACATGGAGCAGGTATCAGTTTAC |
| | | GTTT |
| 183 | s1-11 | GGTTTGTCTGGTCAACCACCGCGTACTCAGTGGTGTACGGT |
| | | ACAAACCAACCACCTGATTGTGATGGATGTTTAGGTTGTTT |
| | | CCAATCTTTTGCTATTAAAAAGAATGTGACAGTGAGAAACT |
| | | TTGTGTATAGTGTCATTTTGCAC |
| 184 | s1-17 | GGTTTGTCTGGTCAACCACCGCGTTCTCAGTGGTGTACGGT |
| | | ACAAACCAGCATCTTTCAAATAGCACCTCATTTTATCCTGA |
| | | AGACCCAG |
| 185 | s1-25 | GGTTTGTCTGGTCAACCACCGCGCACTCAGTGGTGTACGGT |
| | | ACAAACCCAAAACTGGTAGAATCTCAGTGACCTTAAAGATT |
| | | GCCTGGG |
| 186 | s1-28 | GGTTTGTCTGGTCAACCACCGCGGACTCAGTGGTGTACGGT |
| | | ACAAACCCATCTCCCTTTTTCCGTGCTGATTAGGACTGGGG |
| | | CAGTGGGGTTCGTGTATTTGTGACCTGG |
| 187 | s1-31 | GGTTTGTCTGGTCAACCACCGCGACCTCAGTGGTGTACGGT |
| | | ACAAACCGCAGTGTCCACCTGGCGGAACCTTTCTCTCCACT |
| | | ATCCA |
| 188 | s1-39 | GGTTTGTCTGGTCAACCACCGCGGACTCAGTGGTGTACGGT |
| | | ACAAACCTAGTAAATAGGATCAAAGGAATAGAGTCTGATGC |
| | | C |
| 189 | s1-41 | GGTTTGTCTGGTCAACCACCGCGGACTCAGTGGTGTACGGT |
| | | ACAAACCCTGTGACTACATTTAGTGAGCAGGTGGAATGAAC |
| | | AA |
| 190 | s1-43 | GGTTTGTCTGGTCAACCACCGCGTACTCAGTGGTGTACGGT |
| | | ACAAACCGGATAAGTTTCAGTAAAGGGACTACTGCAGGATT |
| | | TGGTGGTA |
| 191 | s3-28 | GGTTTGTCTGGTCAACCACCGCGTCCTCAGTGGTGTACGGT |
| | | ACAAACCTGCGTTGTCTGAGCTCCTCAGCACTCCCTCCCCA |
| | | ACTTCCTAT |
| 192 | s3-41 | GGTTTGTCTGGTCAACCACCGCGAACTCAGTGGTGTACGGT |
| | | ACAAACCAGTCACCAATCTGGAGGGGATGAGGTGAGGGAGG |
| | | GATAGACAATA |

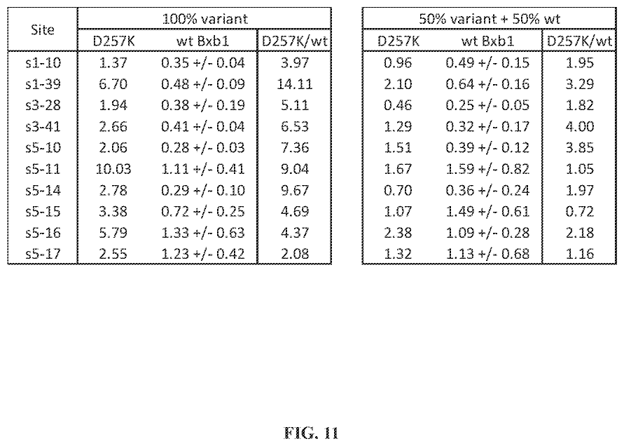

K562 cells were transfected following the manufacturer's protocol. 2E5 cells were transfected with 200 ng of total Bxb1 expression plasmid (200 ng of one Bxb1 expression plasmid, or 100 ng of a Bxb1 variant expression plasmid mixed with 100 ng of a WT Bxb1 expression plasmid), and 1600 ng total of the target site-specific attP donor plasmid. In most experiments, TI activity data for Bxb1 variants was obtained with a mixture of 50% Bxb1 variant expression plasmid and 50% WT Bxb1 expression plasmid. One exception was the data shown in the left panel of FIG. 11, which used 100% (200 ng) Bxb1 D257K expression plasmid or 100% WT Bxb1 expression plasmid. In addition, except for experiments involving the D257K Bxb1 variant, a 50% mixture of Bxb1 variant and WT Bxb1 was used. This is because Bxb1 variants with altered target preferences generally only show improved targeting to half-sites with the alterations shown in Table 1, and may target the attP site in the donor and/or the other half-site more poorly than WT Bxb1. A 50% mixture of variant Bxb1 and WT Bxb1 should allow the Bxb1 variant to bind it's preferred half-site and allow the WT Bxb1 to bind the donor and the half-site not preferred by the Bxb1 variant.

Transfected cells were harvested after three days of incubation at 37° C. using QuickExtract™ by Lucigen (Cat No: QE09050) following the manufacturer's protocol. A polymerase chain reaction (PCR) was performed on the extracted DNA using Invitrogen's Accuprimer™ Taq DNA Polymerase, high fidelity (cat no: 12346094), as shown in Table 6.

The target site-specific PCR was performed using the forward primers shown in Table 15.

TABLE 15
Target Site-Specific Forward Primer Sequences

| SEQ | Site | F-primer |
|---|---|---|
| 193 | s5-1 | ACACGACGCTCTTCCGATCTNNNNTTCTTTCATTTCGTGTG |
| | | AGGGTC |
| 194 | s5-2 | ACACGACGCTCTTCCGATCTNNNNACCCTGTGTTAGAAGCA |
| | | GTAAC |
| 195 | s5-4 | ACACGACGCTCTTCCGATCTNNNNATGTGTTTGGAAGCTGA |
| | | GCATAG |
| 196 | s5-6 | ACACGACGCTCTTCCGATCTNNNNCGATGTGTTCCTCCATG |
| | | AAGCA |
| 197 | s5-8 | ACACGACGCTCTTCCGATCTNNNNGCATAGCTGAGTAATAC |
| | | ATTGCATG |
| 198 | s5-10 | ACACGACGCTCTTCCGATCTNNNNATGGGCAGCATTCATTA |
| | | AGTAAAT |
| 199 | s5-11 | ACACGACGCTCTTCCGATCTNNNNCAATTAGTTGGCTGTAT |
| | | AATTTGG |
| 200 | s5-14 | ACACGACGCTCTTCCGATCTNNNNATTTCACATTGATTGAT |
| | | TTTCTTGTT |
| 201 | s5-15 | ACACGACGCTCTTCCGATCTNNNNACTGCCACCTACATTCA |
| | | ACAAAT |
| 202 | s5-16 | ACACGACGCTCTTCCGATCTNNNNAGAGGGCTGAGGTTGCA |
| | | ATG |
| 203 | s5-17 | ACACGACGCTCTTCCGATCTNNNNTGCAGATAATTCTTATG |
| | | CAAAACAGAAC |
| 204 | s5-18 | ACACGACGCTCTTCCGATCTNNNNGCAGAAAGTTAGTATGA |
| | | CACAGAC |
| 205 | s5-19 | ACACGACGCTCTTCCGATCTNNNNGACCAGACAGTTCCAGA |
| | | TACATTCCA |
| 206 | s5-21 | ACACGACGCTCTTCCGATCTNNNNTTACTCTCTCTCCTAGA |
| | | CTCAAGC |
| 207 | s5-24 | ACACGACGCTCTTCCGATCTNNNNTGCTAGTATTTGTCTGC |
| | | ACTTTCT |
| 208 | s1-1 | ACACGACGCTCTTCCGATCTNNNNCGTGGGCCAGTTTTAAA |
| | | TGTCA |
| 209 | s1-5 | ACACGACGCTCTTCCGATCTNNNNGCTGAACATTAGCTCCA |
| | | TATAT |
| 210 | s1-10 | ACACGACGCTCTTCCGATCTNNNNAGTCTCTCATTTACCAG |
| | | TTTTGCTT |
| 211 | s1-11 | ACACGACGCTCTTCCGATCTNNNNGGATTATCCCATATCCA |
| | | GACATA |
| 212 | s1-17 | ACACGACGCTCTTCCGATCTNNNNAGCCCAGAGTTAACCAA |
| | | GCTAC |
| 213 | s1-25 | ACACGACGCTCTTCCGATCTNNNNTACAACTAAAGCACCAA |
| | | TGGCTC |
| 214 | s1-28 | ACACGACGCTCTTCCGATCTNNNNGTCAGAAGTCCCAGATG |
| | | TGCT |
| 215 | s1-31 | ACACGACGCTCTTCCGATCTNNNNGGAATCCGCCTCCTGAC |
| | | G |
| 216 | s1-39 | ACACGACGCTCTTCCGATCTNNNNTGATGTTAGAGATCAGC |
| | | CTGTAC |
| 217 | s1-41 | ACACGACGCTCTTCCGATCTNNNNAGCCATTTCCTTCCTAG |
| | | CAAATT |
| 218 | s1-43 | ACACGACGCTCTTCCGATCTNNNNCAATACAGGCACAATCC |
| | | CCTTATT |
| 219 | s3-28 | ACACGACGCTCTTCCGATCTNNNNTGAAGGGAAAGGTCTGG |
| | | CTTTA |
| 220 | s3-41 | ACACGACGCTCTTCCGATCTNNNNCAGCAGTCGATGTGGGA |
| | | AC |

The target site-specific PCR was performed using the reverse primers shown in Table 16 below.

TABLE 16
Target Site-Specific Reverse Primer Sequences

| SEQ | Site | R-primer |
|---|---|---|
| 221 | s5-1 | GACGTGTGCTCTTCCGATCTTGTTACATTTGGAGTGAATT |
| | | GGTCA |
| 222 | s5-2 | GACGTGTGCTCTTCCGATCTATATCACTTGCTGCAGGTAT |
| | | ACTTTT |
| 223 | s5-4 | GACGTGTGCTCTTCCGATCTACATGTCAGAAGGGATGGGA |
| | | AG |
| 224 | s5-6 | GACGTGTGCTCTTCCGATCTTAGGCGATGAGGGAAGCTAA |
| | | GT |
| 225 | s5-8 | GACGTGTGCTCTTCCGATCTAGGACCACACAGTTTGAGTA |
| | | GC |
| 226 | s5-10 | GACGTGTGCTCTTCCGATCTAAGTTTTCCCTTCCACAGAA |
| | | A |
| 227 | s5-11 | GACGTGTGCTCTTCCGATCTTCTAAAGAGCAAGTGACAGA |
| | | GAGG |
| 228 | s5-14 | GACGTGTGCTCTTCCGATCTAGAGTAGTGTTGTGCGCCTG |
| 229 | s5-15 | GACGTGTGCTCTTCCGATCTTTTCCTGCTATCTGATTTCA |
| | | AAAGGA |
| 230 | s5-16 | GACGTGTGCTCTTCCGATCTTGCCATCACTCAGAAACAAT |
| | | AATCA |
| 231 | s5-17 | GACGTGTGCTCTTCCGATCTTTACTAGTGGCTCTGGAAAC |
| | | CA |
| 232 | s5-18 | GACGTGTGCTCTTCCGATCTTACTTAATCTGGCCCTGGAG |
| | | ATC |
| 233 | s5-19 | GACGTGTGCTCTTCCGATCTGTCACCCGTACCTCCCCG |
| 234 | s5-21 | GACGTGTGCTCTTCCGATCTGATTTTACCCCTGGAGCTCA |
| | | TG |
| 235 | s5-24 | GACGTGTGCTCTTCCGATCTTCCATAACTCTGTTGGCAAT |
| | | AAG |
| 236 | s1-1 | GACGTGTGCTCTTCCGATCTGCAGCTTAGCCACATGTACC |
| | | TA |
| 237 | s1-5 | GACGTGTGCTCTTCCGATCTCCCTCTGAGTACATTCACTG |
| 238 | s1-10 | GACGTGTGCTCTTCCGATCTAAACGTAAACTGATACCTGC |
| | | TCC |
| 239 | s1-11 | GACGTGTGCTCTTCCGATCTGTGCAAAATGACACTATACA |
| | | CAAAG |
| 240 | s1-17 | GACGTGTGCTCTTCCGATCTCTGGGTCTTCAGGATAAAAT |
| | | GAGG |
| 241 | s1-25 | GACGTGTGCTCTTCCGATCTCCCAGGCAATCTTTAAGGTC |
| | | AC |
| 242 | s1-28 | GACGTGTGCTCTTCCGATCTCCAGGTCACAAATACACGAA |
| | | C |
| 243 | s1-31 | GACGTGTGCTCTTCCGATCTTGGATAGTGGAGAGAAAGGT |
| | | TCC |
| 244 | s1-39 | GACGTGTGCTCTTCCGATCTGGCATCAGACTCTATTCCTT |
| 245 | s1-41 | GACGTGTGCTCTTCCGATCTTTGTTCATTCCACCTGCTCA |
| | | CT |
| 246 | s1-43 | GACGTGTGCTCTTCCGATCTTACCACCAAATCCTGCAGTA |
| | | GT |
| 247 | s3-28 | GACGTGTGCTCTTCCGATCTATAGGAAGTTGGGGAGGGAG |
| | | TG |
| 248 | s3-41 | GACGTGTGCTCTTCCGATCTTATTGTCTATCCCTCCCTCA |
| | | CCT |

The PCR was performed using the temperature cycling protocol in Table 8; the format of the time is minutes: seconds. The product of next generation sequencing (NGS) PCR1 was used as the template for NGS PCR2, as shown in Table 9. The PCR was performed using the temperature cycling protocol in Table 10; the format of the time is minutes: seconds. The primers used are universal primers that add on the required DNA sequences for Illumina Sequencing machines. NGS PCR2 products were purified and sequenced on Illumina Sequencing machines. See also Miller et al., Nat Biotechnol. (2019) 37 (8): 945-52 for further description of this sequencing-based assay.

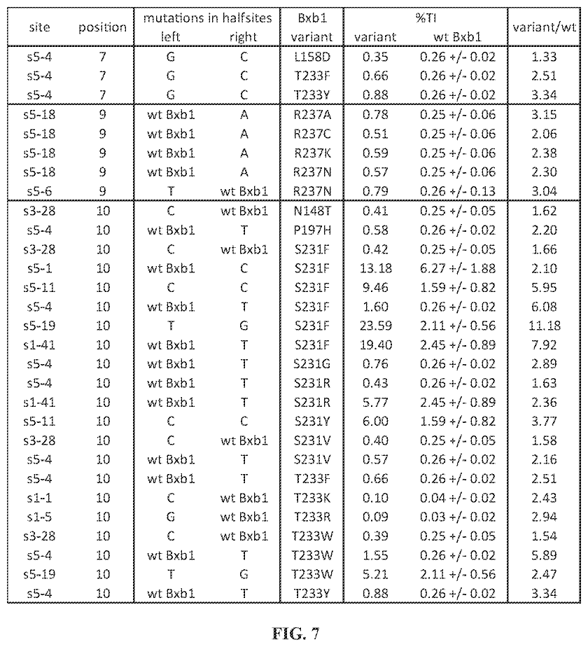

In one example experiment, the target preference shift for Bxb1 variant S231F in position 10 was determined. The S231F variant demonstrated improved targeting of C and T bases, relative to the WT Bxb1 recombinase (FIG. 6A), resulting in improved targeted integration at the endogenous human target sites s5-1, s5-11, and s1-41 (FIGS. 6B-C). The target preference shifts for different Bxb1 variants at positions that bind the RD domain (e.g., positions 7, 9, and 10) are shown in FIG. 7.

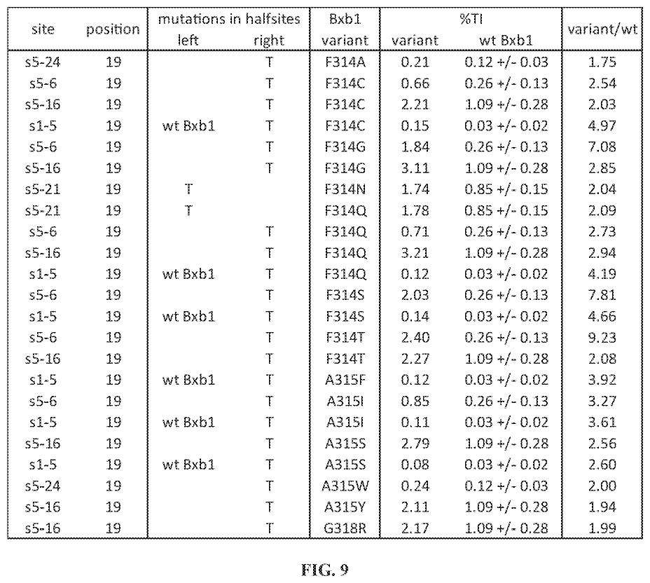

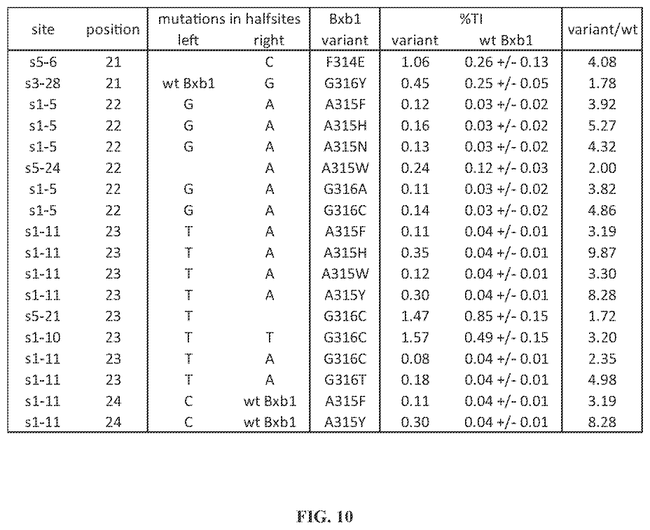

In other example experiments, the target preference shifts for Bxb1 variants F314G and G316Y at positions 19 and 21, respectively, were determined. The F314G variant demonstrated improved targeting of T at position 19, relative to the WT Bxb1, and the G316Y variant demonstrated improved targeting of G at position 21, relative to WT Bxb1 (FIG. 8A). The F314G and G316Y variants were found to have improved targeted integration at the s5-16 and s3-28 endogenous human target sites, respectively (FIGS. 8B-C). The target preference shifts for various Bxb1 variants at positions that bind the ZD domain (e.g., positions 19, 21, 22, 23, and 24) are shown in FIGS. 9 and 10.

The Bxb1 variants described here may also demonstrate improved targeted integration at a variety of different endogenous human target sites, relative to WT Bxb1. For example, the D257K Bxb1 variant demonstrated improved targeted integration at human target sites including s1-10, s1-39, s3-28, s3-41, s5-10, s5-11, s5-14, s5-15, s5-16, and s5-17 (FIG. 11).

### List of Sequences

Sequences disclosed herein are listed in the table below (SEQ: SEQ ID NO).

| SEQ | Description |
|---|---|
| 1 | Wildtype Bxb1 recombinase exemplary amino acid sequence |
| 2 | Wildtype attB sequence |
| 3 | Wildtype attP sequence |
| 4-40 | Variant attB sequences in Table 4 |
| 41-76 | Variant attP sequences in Table 5 |
| 77 | Wildtype ϕC31 integrase exemplary amino acid sequence |
| 78 | Wildtype Pa557 recombinase exemplary amino acid sequence |
| 79 | Wildtype Pa570 recombinase exemplary amino acid sequence |
| 80 | Symmetric attB sequence |
| 81 | Consensus attP sequence |
| 82 | Consensus attB sequence |
| 83-130 | Endogenous human attP- and attB-like sequences in Table 3 |
| 131 | Wildtype LI integrase exemplary amino acid sequence |
| 132 | Wildtype A118 integrase exemplary amino acid sequence |
| 133 | Wildtype TP901 recombinase exemplary amino acid sequence |
| 134 | Consensus attB sequence |
| 135 | Consensus attB sequence |
| 136 | Consensus attB sequence |
| 137-164 | Endogenous Bxb1 pseudo-attB target sequences |
| 165-192 | Target site-specific donor sequences |
| 193-220 | Target site-specific forward primer sequences |
| 221-248 | Target site-specific reverse primer sequences |
| 249 | Bxb1 recombinase construct C terminal end amino acid sequence |
| 250-251 | NGS PCR1 primers in Table 7 |
| 252-254 | Symmetric attB sequences with mutations at position 10 |
| 255-257 | attP sequences with mutations at position 10 |
| 258-260 | Symmetric attB sequences with mutations at position 9 |
| 261-263 | attP sequences with mutations with mutations at position 9 |
| 264-267 | Endogenous Bxb1 pseudo-attB target sequences |
| 268-269 | Symmetric attB sequences |
| 270-271 | Endogenous Bxb1 pseudo-attB target sequences |

What is claimed is:

1. A non-naturally occurring variant of Bxb1 recombinase with altered DNA target specificity relative to wildtype Bxb1 recombinase, comprising one or more amino acid mutations within a zinc ribbon domain (ZD) or a recombinase domain (RD).

2. The Bxb1 recombinase variant of claim 1, wherein the one or more amino acid mutations occur at one or more of positions 147, 148, 149, 154, 155, 156, 158, 197, 198, 230, 231, 232, 233, 237, 257, 309, 312, 314, 315, 316, 318, 323, 324, 325, 326, and 335 (numbering according to SEQ ID NO: 1).

3. The Bxb1 recombinase variant of claim 1, wherein the one or more amino acid mutations are selected from F314A, F314C, F314D, F314E, F314G, F314H, F314I, F314L, F314N, F314Q, F314S, F314T, F314V, F314W, F314Y, A315F, A315G, A315H, A315I, A315M, A315N, A315S, A315T, A315W, A315Y, G316A, G316C, G316D, G316E, G316F, G316H, G316I, G316K, G316L, G316M, G316P, G316Q, G316R, G316S, G316T, G316V, G316W, G316Y, G318I, G318K, G318R, G318W, R323G, R323K, R325D, R325E, R325K, R325L, R325M, R325N, R325Q, R325S, R325W, F147A, F147K, F147R, N148Q, N148T, L158D, L158N, L158S, L158T, L158W, P197H, P197R, P197T, S231F, S231G, S231H, S231K, S231R, S231V, S231Y, T233F, T233H, T233K, T233R, T233W, T233Y, R237A, R237C, R237D, R237E, R237F, R237G, R237H, R237I, R237K, R237L, R237M, R237N, R237P, R237Q, R237S, R237T, R237V, R237W, R237Y, and D257K (numbering according to SEQ ID NO: 1).

4. A non-naturally occurring variant of φC31 integrase, Pa557 recombinase, or Pa570 recombinase, comprising one or more amino acid mutations that produce altered DNA target specificity relative to the wildtype counterpart.

5. The φC31 integrase variant of claim 4, wherein the one or more amino acid mutations occur at one or more of positions 273, 275, 279, 375, 376, 377, 379, and 386 (numbering according to SEQ ID NO: 77).

6. The φC31 integrase variant of claim 5, wherein the one or more amino acid mutations are selected from D273, A275, R279, K375, R376, G377, E379, and R386 (numbering according to SEQ ID NO: 77).

7. The Pa557 recombinase variant of claim 4, wherein the one or more amino acid mutations occur at one or more of positions 236, 238, 242, 327, 328, 329, 331, and 338 (numbering according to SEQ ID NO: 78).

8. The Pa557 recombinase variant of claim 7, wherein the one or more amino acid mutations are selected from G236, A238, A242, R327, T328, G329, G331, and R338 (numbering according to SEQ ID NO: 78).

9. The Pa570 recombinase variant of claim 4, wherein the one or more amino acid mutations occur at one or more of positions 243, 245, 249, 333, 334, 335, 337, and 344 (numbering according to SEQ ID NO: 79).

10. The Pa570 recombinase variant of claim 9, wherein the one or more amino acid mutations are selected from E243, S245, K249, 1333, N334, P335, 1337, and Q344 (numbering according to SEQ ID NO: 79).

11. The recombinase variant of claim 1, wherein the DNA target is specific to a particular allele of a gene.

12. (canceled)

13. (canceled)

14. A nucleic acid molecule encoding the recombinase variant of claim 1.

15. A vector comprising the nucleic acid molecule of claim 14.

16. The vector of claim 15, wherein the vector is a plasmid or a viral vector, optionally selected from an adeno-associated viral vector, an adenoviral vector, or a lentiviral vector.

17. A system for editing DNA in a cell, comprising the recombinase variant of claim 1.

18. (canceled)

19. (canceled)

20. (canceled)

21. (canceled)

22. (canceled)

23. (canceled)

24. (canceled)

25. A method of editing the genome of a cell, the method comprising providing to the cell the system of claim 17.

26. (canceled)

27. (canceled)

28. A cell comprising the system of claim 17, or a descendent thereof.

29. A cell edited by the method of claim 25, or a descendent thereof.

30. (canceled)

31. A method of treating a disease in a subject in need thereof, comprising administering the cell or descendent of claim 29 to the subject.

32. The cell or descendent of claim 29 for use in treating a disease in a subject in need thereof.

33. (canceled)

34. (canceled)


SEQUENCE LISTING (271 sequences)

SEQ ID NO: 1 - protein, 500 aa, synthetic construct [sequence omitted]

SEQ ID NO: 2 - DNA, 38 bp, synthetic construct
  misc_feature 1..38: note = Synthetic: Wildtype attB
  source 1..38: mol_type = other DNA
ggcttgtcgacgacggcggtctccgtcgtcaggatcat

SEQ ID NO: 3 - DNA, 48 bp, synthetic construct
  misc_feature 1..48: note = Synthetic: Wildtype attP
  source 1..48: mol_type = other DNA
ggtttgtctggtcaaccaccgcggtctcagtggtgtacggtacaaacc

SEQ ID NO: 4 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgacgacggcggtctccgtcgtcaacaaaccccggcagataaaagtacccagaaccagagcctacaataccggccctgggaatataag

SEQ ID NO: 5 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggatgtttgtcgacgacggcggtctccgtcgtcaacaaacaccggcagataaaagtacccagaaccagagcctacaataccggccctgggaatataag

SEQ ID NO: 6 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggacgtttgtcgacgacggcggtctccgtcgtcaacaaacgccggcagataaaagtacccagaaccagagcctacaataccggccctgggaatataag

SEQ ID NO: 7 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaagtttgtcgacgacggcggtctccgtcgtcaacaaactccggcagataaaagtacccagaaccagagcctacaataccggccctgggaatataag

SEQ ID NO: 8 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggagttttgtcgacgacggcggtctccgtcgtcaacaaaacccggcagataaaagtacccagaaccagagcctacaataccggccctgggaatataag

SEQ ID NO: 9 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggagctttgtcgacgacggcggtctccgtcgtcaacaaagcccggcagataaaagtacccagaaccagagcctacaataccggccctgggaatataag

SEQ ID NO: 10 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggagatttgtcgacgacggcggtctccgtcgtcaacaaatcccggcagataaaagtacccagaaccagagcctacaataccggccctgggaatataag

SEQ ID NO: 11 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggagggttgtcgacgacggcggtctccgtcgtcaacaacccccggcagataaaagtacccagaaccagagcctacaataccggccctgggaatataag

SEQ ID NO: 12 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggattgtcgacgacggcggtctccgtcgtcaacaatccccggcagataaaagtacccagaaccagagcctacaataccggccctgggaatataag

SEQ ID NO: 13 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggcttgtcgacgacggcggtctccgtcgtcaacaagccccggcagataaaagtacccagaaccagagcctacaataccggccctgggaatataag

SEQ ID NO: 14 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtgtgtcgacgacggcggtctccgtcgtcaacacaccccggcagataaaagtacccagaaccagagcctacaataccggccctgggaatataag

SEQ ID NO: 15 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtatgtcgacgacggcggtctccgtcgtcaacataccccggcagataaaagtacccagaaccagagcctacaataccggccctgggaatataag

SEQ ID NO: 16 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtctgtcgacgacggcggtctccgtcgtcaacagaccccggcagataaaagtacccagaaccagagcctacaataccggccctgggaatataag

SEQ ID NO: 17 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggttggtcgacgacggcggtctccgtcgtcaaccaaccccggcagataaaagtacccagaaccagagcctacaataccggccctgggaatataag

SEQ ID NO: 18 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggttagtcgacgacggcggtctccgtcgtcaactaaccccggcagataaaagtacccagaaccagagcctacaataccggccctgggaatataag

SEQ ID NO: 19 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggttcgtcgacgacggcggtctccgtcgtcaacgaaccccggcagataaaagtacccagaaccagagcctacaataccggccctgggaatataag

SEQ ID NO: 20 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttttcgacgacggcggtctccgtcgtcaaaaaaccccggcagataaaagtacccagaaccagagcctacaataccggccctgggaatataag

SEQ ID NO: 21 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttctcgacgacggcggtctccgtcgtcaagaaaccccggcagataaaagtacccagaaccagagcctacaataccggccctgggaatataag

SEQ ID NO: 22 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttatcgacgacggcggtctccgtcgtcaataaaccccggcagataaaagtacccagaaccagagcctacaataccggccctgggaatataag

SEQ ID NO: 23 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttggcgacgacggcggtctccgtcgtcaccaaaccccggcagataaaagtacccagaaccagagcctacaataccggccctgggaatataag

SEQ ID NO: 24 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgacgacgacggcggtctccgtcgtcatcaaaccccggcagataaaagtacccagaaccagagcctacaataccggccctgggaatataag

SEQ ID NO: 25 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgccgacgacggcggtctccgtcgtcagcaaaccccggcagataaaagtacccagaaccagagcctacaataccggccctgggaatataag

SEQ ID NO: 26 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgtcgacggcggtctccgtcgacaacaaaccccggcagataaaagtacccagaaccagagcctacaataccggccctgggaatataag

SEQ ID NO: 27 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcggcgacggcggtctccgtcgccaacaaaccccggcagataaaagtacccagaaccagagcctacaataccggccctgggaatataag

SEQ ID NO: 28 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgccgacggcggtctccgtcggcaacaaaccccggcagataaaagtacccagaaccagagcctacaataccggccctgggaatataag

SEQ ID NO: 29 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgaggacggcggtctccgtcctcaacaaaccccggcagataaaagtacccagaaccagagcctacaataccggccctgggaatataag

SEQ ID NO: 30 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgatgacggcggtctccgtcatcaacaaaccccggcagataaaagtacccagaaccagagcctacaataccggccctgggaatataag

SEQ ID NO: 31 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgaagacggcggtctccgtcttcaacaaaccccggcagataaaagtacccagaaccagagcctacaataccggccctgggaatataag

SEQ ID NO: 32 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgactacggcggtctccgtagtcaacaaaccccggcagataaaagtacccagaaccagagcctacaataccggccctgggaatataag

SEQ ID NO: 33 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgaccacggcggtctccgtggtcaacaaaccccggcagataaaagtacccagaaccagagcctacaataccggccctgggaatataag

SEQ ID NO: 34 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgacaacggcggtctccgttgtcaacaaaccccggcagataaaagtacccagaaccagagcctacaataccggccctgggaatataag

SEQ ID NO: 35 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgacgtcggcggtctccgacgtcaacaaaccccggcagataaaagtacccagaaccagagcctacaataccggccctgggaatataag

SEQ ID NO: 36 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgacggcggcggtctccgccgtcaacaaaccccggcagataaaagtacccagaaccagagcctacaataccggccctgggaatataag

SEQ ID NO: 37 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgacgccggcggtctccggcgtcaacaaaccccggcagataaaagtacccagaaccagagcctacaataccggccctgggaatataag

SEQ ID NO: 38 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgacgagggcggtctccctcgtcaacaaaccccggcagataaaagtacccagaaccagagcctacaataccggccctgggaatataag

SEQ ID NO: 39 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgacgatggcggtctccatcgtcaacaaaccccggcagataaaagtacccagaaccagagcctacaataccggccctgggaatataag

SEQ ID NO: 40 - DNA, 137 bp, synthetic construct
  misc_feature 1..137: note = Synthetic: Variant attB sequence
  source 1..137: mol_type = other DNA
atgcttctctgcatccgtctgctaaatccgcgtgataggggaggtttgtcgacgaaggcggtctccttcgtcaacaaaccccggcagataaaagtacccagaaccagagcctacaataccggccctgggaatataag

SEQ ID NO: 41 - DNA, 100 bp, synthetic construct
  misc_feature 1..100: note = Synthetic: Variant attP sequence
  source 1..100: mol_type = other DNA
ggtttgtctggtcatccaccgcggtctcagtggagtacggtacaaacccaataaaagtacccagaaccagagcctacaataccggccctgggaatataag

SEQ ID NO: 42 - DNA, 100 bp, synthetic construct
  misc_feature 1..100: note = Synthetic: Variant attP sequence
  source 1..100: mol_type = other DNA
ggtttgtctggtcacccaccgcggtctcagtggggtacggtacaaacccaataaaagtacccagaaccagagcctacaataccggccctgggaatataag

SEQ ID NO: 43 - DNA, 100 bp, synthetic construct
  misc_feature 1..100: note = Synthetic: Variant attP sequence
  source 1..100: mol_type = other DNA
ggtttgtctggtcagccaccgcggtctcagtggcgtacggtacaaacccaataaaagtacccagaaccagagcctacaataccggccctgggaatataag

SEQ ID NO: 44 - DNA, 100 bp, synthetic construct
  misc_feature 1..100: note = Synthetic: Variant attP sequence
  source 1..100: mol_type = other DNA
ggtttgtctggtcaaacaccgcggtctcagtgttgtacggtacaaacccaataaaagtacccagaaccagagcctacaataccggccctgggaatataag

SEQ ID NO: 45 - DNA, 100 bp, synthetic construct
  misc_feature 1..100: note = Synthetic: Variant attP sequence
  source 1..100: mol_type = other DNA
ggtttgtctggtcaatcaccgcggtctcagtgatgtacggtacaaacccaataaaagtacccagaaccagagcctacaataccggccctgggaatataag

SEQ ID NO: 46 - DNA, 100 bp, synthetic construct
  misc_feature 1..100: note = Synthetic: Variant attP sequence
  source 1..100: mol_type = other DNA
ggtttgtctggtcaagcaccgcggtctcagtgctgtacggtacaaacccaataaaagtacccagaaccagagcctacaataccggccctgggaatataag

SEQ ID NO: 47 - DNA, 100 bp, synthetic construct
  misc_feature 1..100: note = Synthetic: Variant attP sequence
  source 1..100: mol_type = other DNA
ggtttgtctggtcaactaccgcggtctcagtagtgtacggtacaaacccaataaaagtacccagaaccagagcctacaataccggccctgggaatataag

SEQ ID NO: 48 - DNA, 100 bp, synthetic construct
  misc_feature 1..100: note = Synthetic: Variant attP sequence
  source 1..100: mol_type = other DNA
ggtttgtctggtcaacaaccgcggtctcagttgtgtacggtacaaacccaataaaagtacccagaaccagagcctacaataccggccctgggaatataag

SEQ ID NO: 49 - DNA, 100 bp, synthetic construct
  misc_feature 1..100: note = Synthetic: Variant attP sequence
  source 1..100: mol_type = other DNA
ggtttgtctggtcaacgaccgcggtctcagtcgtgtacggtacaaacccaataaaagtacccagaaccagagcctacaataccggccctgggaatataag

SEQ ID NO: 50 - DNA, 100 bp, synthetic construct
  misc_feature 1..100: note = Synthetic: Variant attP sequence
  source 1..100: mol_type = other DNA
ggtttgtctggtcaacctccgcggtctcagaggtgtacggtacaaacccaataaaagtacccagaaccagagcctacaataccggccctgggaatataag

SEQ ID NO: 51 - DNA, 100 bp, synthetic construct
  misc_feature 1..100: note = Synthetic: Variant attP sequence
  source 1..100: mol_type = other DNA
ggtttgtctggtcaacccccgcggtctcaggggtgtacggtacaaacccaataaaagtacccagaaccagagcctacaataccggccctgggaatataag

SEQ ID NO: 52 - DNA, 100 bp, synthetic construct
  misc_feature 1..100: note = Synthetic: Variant attP sequence
  source 1..100: mol_type = other DNA
ggtttgtctggtcaaccgccgcggtctcagcggtgtacggtacaaacccaataaaagtacccagaaccagagcctacaataccggccctgggaatataag

SEQ ID NO: 53 - DNA, 100 bp, synthetic construct
  misc_feature 1..100: note = Synthetic: Variant attP sequence
  source 1..100: mol_type = other DNA
ggtttgtctggtcaaccagcgcggtctcactggtgtacggtacaaacccaataaaagtacccagaaccagagcctacaataccggccctgggaatataag

SEQ ID NO: 54 - DNA, 100 bp, synthetic construct
  misc_feature 1..100: note = Synthetic: Variant attP sequence
  source 1..100: mol_type = other DNA
ggtttgtctggtcaaccatcgcggtctcaatggtgtacggtacaaacccaataaaagtacccagaaccagagcctacaataccggccctgggaatataag

SEQ ID NO: 55 - DNA, 100 bp, synthetic construct
  misc_feature 1..100: note = Synthetic: Variant attP sequence
  source 1..100: mol_type = other DNA
ggtttgtctggtcaaccaacgcggtctcattggtgtacggtacaaacccaataaaagtacccagaaccagagcctacaataccggccctgggaatataag

SEQ ID NO: 56 - DNA, 100 bp, synthetic construct
  misc_feature 1..100: note = Synthetic: Variant attP sequence
  source 1..100: mol_type = other DNA
cgtttgtctggtcaaccaccgcggtctcagtggtgtacggtacaaacgcaataaaagtacccagaaccagagcctacaataccggccctgggaatataag

SEQ ID NO: 57 - DNA, 100 bp, synthetic construct
  misc_feature 1..100: note = Synthetic: Variant attP sequence
  source 1..100: mol_type = other DNA
tgtttgtctggtcaaccaccgcggtctcagtggtgtacggtacaaacacaataaaagtacccagaaccagagcctacaataccggccctgggaatataag

SEQ ID NO: 58 - DNA, 100 bp, synthetic construct
  misc_feature 1..100: note = Synthetic: Variant attP sequence
  source 1..100: mol_type = other DNA
agtttgtctggtcaaccaccgcggtctcagtggtgtacggtacaaactcaataaaagtacccagaaccagagcctacaataccggccctgggaatataag

SEQ ID NO: 59 - DNA, 100 bp, synthetic construct
  misc_feature 1..100: note = Synthetic: Variant attP sequence
  source 1..100: mol_type = other DNA
gctttgtctggtcaaccaccgcggtctcagtggtgtacggtacaaagccaataaaagtacccagaaccagagcctacaataccggccctgggaatataag

SEQ ID NO: 60 - DNA, 100 bp, synthetic construct
  misc_feature 1..100: note = Synthetic: Variant attP sequence
  source 1..100: mol_type = other DNA
gttttgtctggtcaaccaccgcggtctcagtggtgtacggtacaaaaccaataaaagtacccagaaccagagcctacaataccggccctgggaatataag

SEQ ID NO: 61 - DNA, 100 bp, synthetic construct
  misc_feature 1..100: note = Synthetic: Variant attP sequence
  source 1..100: mol_type = other DNA
gatttgtctggtcaaccaccgcggtctcagtggtgtacggtacaaatccaataaaagtacccagaaccagagcctacaataccggccctgggaatataag

SEQ ID NO: 62 - DNA, 100 bp, synthetic construct
  misc_feature 1..100: note = Synthetic: Variant attP sequence
  source 1..100: mol_type = other DNA
ggattgtctggtcaaccaccgcggtctcagtggtgtacggtacaatcccaataaaagtacccagaaccagagcctacaataccggccctgggaatataag

SEQ ID NO: 63 - DNA, 100 bp, synthetic construct
  misc_feature 1..100: note = Synthetic: Variant attP sequence
  source 1..100: mol_type = other DNA
ggcttgtctggtcaaccaccgcggtctcagtggtgtacggtacaagcccaataaaagtacccagaaccagagcctacaataccggccctgggaatataag

SEQ ID NO: 64 - DNA, 100 bp, synthetic construct
  misc_feature 1..100: note = Synthetic: Variant attP sequence
  source 1..100: mol_type = other DNA
gggttgtctggtcaaccaccgcggtctcagtggtgtacggtacaaccccaataaaagtacccagaaccagagcctacaataccggccctgggaatataag

SEQ ID NO: 65 - DNA, 100 bp, synthetic construct
  misc_feature 1..100: note = Synthetic: Variant attP sequence
  source 1..100: mol_type = other DNA
ggtctgtctggtcaaccaccgcggtctcagtggtgtacggtacagacccaataaaagtacccagaaccagagcctacaataccggccctgggaatataag

SEQ ID NO: 66 - DNA, 100 bp, synthetic construct
  misc_feature 1..100: note = Synthetic: Variant attP sequence
  source 1..100: mol_type = other DNA
ggtgtgtctggtcaaccaccgcggtctcagtggtgtacggtacacacccaataaaagtacccagaaccagagcctacaataccggccctgggaatataag

SEQ ID NO: 67 - DNA, 100 bp, synthetic construct
  misc_feature 1..100: note = Synthetic: Variant attP sequence
  source 1..100: mol_type = other DNA
ggtatgtctggtcaaccaccgcggtctcagtggtgtacggtacatacccaataaaagtacccagaaccagagcctacaataccggccctgggaatataag

SEQ ID NO: 68 - DNA, 100 bp, synthetic construct
  misc_feature 1..100: note = Synthetic: Variant attP sequence
  source 1..100: mol_type = other DNA
ggttagtctggtcaaccaccgcggtctcagtggtgtacggtactaacccaataaaagtacccagaaccagagcctacaataccggccctgggaatataag

SEQ ID NO: 69 - DNA, 100 bp, synthetic construct
  misc_feature 1..100: note = Synthetic: Variant attP sequence
  source 1..100: mol_type = other DNA
ggttcgtctggtcaaccaccgcggtctcagtggtgtacggtacgaacccaataaaagtacccagaaccagagcctacaataccggccctgggaatataag

SEQ ID NO: 70 - DNA, 100 bp, synthetic construct
  misc_feature 1..100: note = Synthetic: Variant attP sequence
  source 1..100: mol_type = other DNA
ggttggtctggtcaaccaccgcggtctcagtggtgtacggtaccaacccaataaaagtacccagaaccagagcctacaataccggccctgggaatataag

SEQ ID NO: 71 - DNA, 100 bp, synthetic construct
  misc_feature 1..100: note = Synthetic: Variant attP sequence
  source 1..100: mol_type = other DNA
ggtttttctggtcaaccaccgcggtctcagtggtgtacggtaaaaacccaataaaagtacccagaaccagagcctacaataccggccctgggaatataag

SEQ ID NO: 72 - DNA, 100 bp, synthetic construct
  misc_feature 1..100: note = Synthetic: Variant attP sequence
  source 1..100: mol_type = other DNA
ggtttatctggtcaaccaccgcggtctcagtggtgtacggtataaacccaataaaagtacccagaaccagagcctacaataccggccctgggaatataag

SEQ ID NO: 73 - DNA, 100 bp, synthetic construct
  misc_feature 1..100: note = Synthetic: Variant attP sequence
  source 1..100: mol_type = other DNA
ggtttctctggtcaaccaccgcggtctcagtggtgtacggtagaaacccaataaaagtacccagaaccagagcctacaataccggccctgggaatataag

SEQ ID NO: 74 - DNA, 100 bp, synthetic construct
  misc_feature 1..100: note = Synthetic: Variant attP sequence
  source 1..100: mol_type = other DNA
ggtttgactggtcaaccaccgcggtctcagtggtgtacggttcaaacccaataaaagtacccagaaccagagcctacaataccggccctgggaatataag

SEQ ID NO: 75 - DNA, 100 bp, synthetic construct
  misc_feature 1..100: note = Synthetic: Variant attP sequence
  source 1..100: mol_type = other DNA
ggtttgcctggtcaaccaccgcggtctcagtggtgtacggtgcaaacccaataaaagtacccagaaccagagcctacaataccggccctgggaatataag

SEQ ID NO: 76 - DNA, 100 bp, synthetic construct
  misc_feature 1..100: note = Synthetic: Variant attP sequence
  source 1..100: mol_type = other DNA
ggtttggctggtcaaccaccgcggtctcagtggtgtacggtccaaacccaataaaagtacccagaaccagagcctacaataccggccctgggaatataag

SEQ ID NO: 77 - protein, 605 aa, synthetic construct
  REGION 1..605: note = Synthetic: Wildtype phi-C31
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 78 - protein, 557 aa, synthetic construct
  REGION 1..557: note = Synthetic: Wildtype Pa557
MNMHSPTVTTRAALYLRVSTARQAEHDISIPDQKRQGEAYCEQRGFQLVETYVEPGATATNDKRPEFQRMIEAGTSKPAPFDIVVVHSFSRFFRDHFEMEFYVRKLAKNGVKLVSITQEMGDDPMHQMMRQIMALFDEYQSKENAKHVLRAMNENARQGFWNGARPPIGYRIVAAEQRGSKTKKKLEIDPLHADTVRLIYRLFLEGDGTRGAMGVKAIATYLNERRFFTRDGGRWGLAQIHAILTRTTYIGEHRFNTRSHKDREKKPESEIAIMAVPPLIEREIYDAVQARLKSRNPMVTPARVSSGPTLLTGICFCAKCGGAMTLRTGQGSTGATYRYYTCSTKARQGKTGCKGRTIPMDKLDHLVADHIGDRLLQPKRLETVLASVIDRRQERAERRREHLAELNRRITEADQRLGRLFDAIEAGMVDKDDAMAKERMVSLKALRDQAAADAERTQLALDSSGNQGVSPDMLKGFARKARERIRLDDGGYRRDHLRALAQRVEVADDEVRIMGSKSELLRTLVAASSVETAAFGVQSSVLKWRTQEDSNLRPLGS

SEQ ID NO: 79 - protein, 570 aa, synthetic construct
  REGION 1..570: note = Synthetic: Wildtype Pa570
MARLISYLRMSTSEQLRGFSLERQRKLIADFAAKNGLSVEEENTLEDIGRSSFSDDAQQKELTRFFENLNAGKYEPGDVFALENIDRLTRRGPVDAILKVNQIISKGLKLAIISGNEQRIIEDVNDVFTIINLSIDASRANKESKNKSDKGLSNWQEKRNLASTYKIAMTAQAPAWLDTEIFYIFDEEKKKNTKRRKYVLNEEKAEAVRLIFDLYSNGNGALKIKNILNERNIPTFKGAPYWEPSIITKILKNPATFGLYQPKKQGTGKRDLIAAGEPINDYFPPVITRDLFEQCEHIREGNSTRKGRKGKLFTNLFTGLLTCSKCGGPVHLINPGIDKRNKVQKSIYYLVCKRAKFTKECTTKRVRYDDFEIALLKAIQEINLADILNENNPLEILVKKQRSKETEINKKRKLIENFQRQFLENDGDLPSFMISQAKDAEISIKELEEDQREIASEIAQLNIYNSNVDNAIEELKENADYGTRSKINLLFHEIIKNISLDTENQFYTVRFKNGVMRVITAAGFIATTEEQTQADINAILQSIEGPRIPREIATDAEKLIEYLKAREVIE

SEQ ID NO: 80 - DNA, 38 bp, synthetic construct
  misc_feature 1..38: note = Synthetic: Symmetric attB
  source 1..38: mol_type = other DNA
ggtttgtcgacgacggcggtctccgtcgtcaacaaacc

SEQ ID NO: 81 - DNA, 48 bp, synthetic construct
  misc_feature 1..48: note = Synthetic: attP Consensus
  source 1..48: mol_type = other DNA
ggtttgtnnnnnnnacnacngnnnnnncngtngtnnnnnnnacaaacc

SEQ ID NO: 82 - DNA, 38 bp, synthetic construct
  misc_feature 1..38: note = Synthetic: attB Consensus
  source 1..38: mol_type = other DNA
ggtttgtnnacnacngnnnnnncngtngtnnacaaacc

SEQ ID NO: 83 - DNA, 38 bp, synthetic construct
  misc_feature 1..38: note = Synthetic: chr18 - 23228819
  source 1..38: mol_type = other DNA
tgcttgttgcctacagcctctgcagaagttcacaaact

SEQ ID NO: 84 - DNA, 38 bp, synthetic construct
  misc_feature 1..38: note = Synthetic: chr21 - 39796718
  source 1..38: mol_type = other DNA
gggttgttgactacagattgcactattttgcagaaacc

SEQ ID NO: 85 - DNA, 38 bp, synthetic construct
  misc_feature 1..38: note = Synthetic: chr5 - 130870404
  source 1..38: mol_type = other DNA
tagttgtagaccacagaaatttctgctgttcagaaact

SEQ ID NO: 86 - DNA, 38 bp, synthetic construct
  misc_feature 1..38: note = Synthetic: chr10 - 114431581
  source 1..38: mol_type = other DNA
gttttgtggacaatggtttcctctgctgtaaagaaacg

SEQ ID NO: 87 - DNA, 38 bp, synthetic construct
  misc_feature 1..38: note = Synthetic: chr4 - 78331600
  source 1..38: mol_type = other DNA
gggttggtggcaatggagatctcagtggtgaaaaatca

SEQ ID NO: 88 - DNA, 38 bp, synthetic construct
  misc_feature 1..38: note = Synthetic: chr3 - 21564386
  source 1..38: mol_type = other DNA
agcttgtttacatgtgaattttctgtagtaaacaacca

SEQ ID NO: 89 - DNA, 38 bp, synthetic construct
  misc_feature 1..38: note = Synthetic: chr17 - 37733272
  source 1..38: mol_type = other DNA
tgtttgtactctaccgaagattccatagttaacaacca

SEQ ID NO: 90 - DNA, 38 bp, synthetic construct
  misc_feature 1..38: note = Synthetic: chr7 - 37683631
  source 1..38: mol_type = other DNA
tatttcttgacaattggcctctctgtagtttgcaacct

SEQ ID NO: 91 - DNA, 38 bp, synthetic construct
  misc_feature 1..38: note = Synthetic: chr9 - 94861248
  source 1..38: mol_type = other DNA
ggtttgagggcagcagcagcagctgctgtcaccaaaca

SEQ ID NO: 92 - DNA, 38 bp, synthetic construct
  misc_feature 1..38: note = Synthetic: chrX - 50461727
  source 1..38: mol_type = other DNA
gttttgttcactgtagaatcctcagtggttagcacaat

SEQ ID NO: 93 - DNA, 38 bp, synthetic construct
  misc_feature 1..38: note = Synthetic: chr1 - 203596869
  source 1..38: mol_type = other DNA
ctcttgatgactgcagagtattccattgttgacaaatc

SEQ ID NO: 94 - DNA, 38 bp, synthetic construct
  misc_feature 1..38: note = Synthetic: chr5 - 35969016
  source 1..38: mol_type = other DNA
tgtttctcaactacagcatgagctgtggttgacagtaa

SEQ ID NO: 95 - DNA, 38 bp, synthetic construct
  misc_feature 1..38: note = Synthetic: chr1 - 183975066
  source 1..38: mol_type = other DNA
gatttggatacacctgagatgacagttgtcaagaaatc

SEQ ID NO: 96 - DNA, 38 bp, synthetic construct
  misc_feature 1..38: note = Synthetic: chr6 - 120401383
  source 1..38: mol_type = other DNA
atttgtttaactactgagttttcagtagttaaaaagct

SEQ ID NO: 97 - DNA, 38 bp, synthetic construct
  misc_feature 1..38: note = Synthetic: chr2 - 131113644
  source 1..38: mol_type = other DNA
ggctttttcattacagaacacccagttgtcagtatcag

SEQ ID NO: 98 - DNA, 38 bp, synthetic construct
  misc_feature 1..38: note = Synthetic: chr18 - 68095661
  source 1..38: mol_type = other DNA
ttattgttgacaactgatatttccacggtgagaatccc

SEQ ID NO: 99 - DNA, 38 bp, synthetic construct
  misc_feature 1..38: note = Synthetic: chr10 - 95438042
  source 1..38: mol_type = other DNA
gggtttttaaacactgacttgcctggtgtgaggatcca

SEQ ID NO: 100 - DNA, 38 bp, synthetic construct
  misc_feature 1..38: note = Synthetic: chr11 - 93987223
  source 1..38: mol_type = other DNA
aatttgctgacaacaggtgcttctgtggttaggagtgg

SEQ ID NO: 101 - DNA, 38 bp, synthetic construct
  misc_feature 1..38: note = Synthetic: chr18 - 38351472
  source 1..38: mol_type = other DNA
ggcttttagactgatgagtgatcagtcttcaggatccc

SEQ ID NO: 102 - DNA, 38 bp, synthetic construct
  misc_feature 1..38: note = Synthetic: chr6 - 84802299
  source 1..38: mol_type = other DNA
atagtgtttacctcagaaagatctgtggtaaggaccaa

SEQ ID NO: 103 - DNA, 38 bp, synthetic construct
  misc_feature 1..38: note = Synthetic: chrX - 54472830
  source 1..38: mol_type = other DNA
ggtgtgaggacaagagagtgagcagtagtcaagagctc

SEQ ID NO: 104 - DNA, 38 bp, synthetic construct
  misc_feature 1..38: note = Synthetic: chr3 - 138022040
  source 1..38: mol_type = other DNA
tgtatgtggaccataggaaaagctggagtcaggagctc

SEQ ID NO: 105 - DNA, 38 bp, synthetic construct
  misc_feature 1..38: note = Synthetic: chr7 - 125717751
  source 1..38: mol_type = other DNA
tgtttgtggaggagtgatggctcagtggtatggatcta

SEQ ID NO: 106 - DNA, 38 bp, synthetic construct
  misc_feature 1..38: note = Synthetic: chr6 - 29957229
  source 1..38: mol_type = other DNA
gcttttttgacctcggcctccgctcaggtcaggaccag

SEQ ID NO: 107 - DNA, 38 bp, synthetic construct
  misc_feature 1..38: note = Synthetic: chr2 - 118918350
  source 1..38: mol_type = other DNA
ctgatacttgcaactgaacacactgtagtccacacacc

SEQ ID NO: 108 - DNA, 38 bp, synthetic construct
  misc_feature 1..38: note = Synthetic: chr1 - 218729444
  source 1..38: mol_type = other DNA
gagatcttggcaataggaacctcagctgtcagcaagca

SEQ ID NO: 109 - DNA, 38 bp, synthetic construct
  misc_feature 1..38: note = Synthetic: chr12 - 82686460
  source 1..38: mol_type = other DNA
gagtttgtgacaaccgaagctgcagcagtcgccaaacc

SEQ ID NO: 110 - DNA, 38 bp, synthetic construct
  misc_feature 1..38: note = Synthetic: chr1 - 12564817
  source 1..38: mol_type = other DNA
ttgcttctcaccgtagatgaccctcttgtccacaaacc

SEQ ID NO: 111 - DNA, 38 bp, synthetic construct
  misc_feature 1..38: note = Synthetic: chr14 - 33253493
  source 1..38: mol_type = other DNA
cagctcctcacaactgatttcccaatggtaatcaagct

SEQ ID NO: 112 - DNA, 38 bp, synthetic construct
  misc_feature 1..38: note = Synthetic: chr5 - 168835161
  source 1..38: mol_type = other DNA
gagatcctagccacaggcgggcccagggttggcaaacc

SEQ ID NO: 113 - DNA, 38 bp, synthetic construct
  misc_feature 1..38: note = Synthetic: chr1 - 205211683
  source 1..38: mol_type = other DNA
tggctcccaacctccgtcactgccgttgcaaacaaacc

SEQ ID NO: 114 - DNA, 38 bp, synthetic construct
  misc_feature 1..38: note = Synthetic: chr17 - 51831373
  source 1..38: mol_type = other DNA
agattcctgactacagtggatgctgttgtttgcaattc

SEQ ID NO: 115 - DNA, 38 bp, synthetic construct
  misc_feature 1..38: note = Synthetic: chr7 - 73438826
  source 1..38: mol_type = other DNA
cagctcctgaacacagcagtcccaggtgttgaaaagcc

SEQ ID NO: 116 - DNA, 38 bp, synthetic construct
  misc_feature 1..38: note = Synthetic: chr8 - 138569746
  source 1..38: mol_type = other DNA
ttggtctttacaactggggctactgtggtggataatcg

SEQ ID NO: 117 - DNA, 38 bp, synthetic construct
  misc_feature 1..38: note = Synthetic: chr7 - 26280167
  source 1..38: mol_type = other DNA
caggtcctgacctcagagccagctggaatgaaaaaacc

SEQ ID NO: 118 - DNA, 38 bp, synthetic construct
  misc_feature 1..38: note = Synthetic: chr18 - 64064874
  source 1..38: mol_type = other DNA
cagatgctctccacagggtctgctgcagtttacaatcc

SEQ ID NO: 119 - DNA, 38 bp, synthetic construct
  misc_feature 1..38: note = Synthetic: chr15 - 26397212
  source 1..38: mol_type = other DNA
atgatcctcactacagctatggcaattgtattgatcct

SEQ ID NO: 120 - DNA, 38 bp, synthetic construct
  misc_feature 1..38: note = Synthetic: chr11 - 121591710
  source 1..38: mol_type = other DNA
gtgctcctaacagcaggcggaacagaggtgaggatcct

SEQ ID NO: 121 - DNA, 38 bp, synthetic construct
  misc_feature 1..38: note = Synthetic: chr1 - 84080800
  source 1..38: mol_type = other DNA
aagatcctgacctccgaagaagctggagttgagatcat

SEQ ID NO: 122 - DNA, 38 bp, synthetic construct
  misc_feature 1..38: note = Synthetic: chr6 - 124844046
  source 1..38: mol_type = other DNA
atggtcccagctactggggaggctgaggtgaggatcgc

SEQ ID NO: 123 - DNA, 38 bp, synthetic construct
  misc_feature 1..38: note = Synthetic: chr3 - 194380608
  source 1..38: mol_type = other DNA
ttgctcctcacagcagaggactctgaagacaggagcat

SEQ ID NO: 124 - DNA, 38 bp, synthetic construct
  misc_feature 1..38: note = Synthetic: chr21 - 38902753
  source 1..38: mol_type = other DNA
tggatcttcactatggcactcccattggtatggatctc

SEQ ID NO: 125 - DNA, 38 bp, synthetic construct
  misc_feature 1..38: note = Synthetic: chr18 - 33954365
  source 1..38: mol_type = other DNA
cagatctatacggctgactacacagtggtgagaaccat

SEQ ID NO: 126 - DNA, 38 bp, synthetic construct
  misc_feature 1..38: note = Synthetic: chr14 - 42626161
  source 1..38: mol_type = other DNA
atgatcctcacagcagatatatctgttctcaagattta

SEQ ID NO: 127 - DNA, 38 bp, synthetic construct
  misc_feature 1..38: note = Synthetic: chr10 - 27365656
  source 1..38: mol_type = other DNA
atgatcttgacaacagggagtgctgggttctggaacat

SEQ ID NO: 128 - DNA, 38 bp, synthetic construct
  misc_feature 1..38: note = Synthetic: chr4 - 9830819
  source 1..38: mol_type = other DNA
ctgatgctgaccacagcaaatgctgccaacaggatccc

SEQ ID NO: 129 - DNA, 38 bp, synthetic construct
  misc_feature 1..38: note = Synthetic: chr12 - 69787548
  source 1..38: mol_type = other DNA
atgatcctaactcttgctttatctactgtgaaaatcat

SEQ ID NO: 130 - DNA, 38 bp, synthetic construct
  misc_feature 1..38: note = Synthetic: chr11 - 97082317
  source 1..38: mol_type = other DNA
ttggtcctggatcctgaggttgctgtggtgaagatccc

SEQ ID NO: 131 - protein, 452 aa, synthetic construct
  REGION 1..452: note = Synthetic: Wildtype LI
MKAAIYIRVSTQEQVENYSIQAQTEKLTALCRSKDWDVYDTFIDGGYSGSNMNRPALNEMLSKLHEIDAVVVYRLDRLSRSQKDTITLIEEYFLKNNVEFVSLSETLDTSSPFGRAMIGILSVFAQLERETIRDRMVMGKIKRIEAGLPLTTAKGRTFGYDVIDTKLYINEEEAKQLRLIYDIFEEEQSITFLQKRLKKLGFKVRTYNRYNNWLTNDLYCGYVSYKDKVHVKGIHEPIISEEQFYRVQEIFSRMGKNPNMNKESASLLNNLVVCSKCGLGFVHRRKDTVSRGKKYHYRYYSCKTYKHTHELEKCGNKIWRADKLEELIIDRVNNYSFASRNIDKEDELDSLNEKLKIEHAKKKRLFDLYINGSYEVSELDSMMNDIDAQINYYEAQIEANEELKKNKKIQENLADLATVDFNSLEFREKQLYLKSLINKIYIDGEQVTIEWL

SEQ ID NO: 132 - protein, 452 aa, synthetic construct
  REGION 1..452: note = Synthetic: Wildtype A118
MKAAIYIRVSTQEQVENYSIQAQTEKLTALCRSKDWDVYDIFIDGGYSGSNMNRPALNEMLSKLHEIDAVVVYRLDRLSRSQRDTITLIEEYFLKNNVEFVSLSETLDTSSPFGRAMIGILSVFAQLERETIRDRMVMGKIKRIEAGLPLTTAKGRTFGYDVIDTKLYINEEEAKQLQLIYDIFEEEQSITFLQKRLKKLGFKVRTYNRYNNWLTNDLYCGYVSYKDKVHVKGIHEPIISEEQFYRVQEIFTRMGKNPNMNRDSASLLNNLVVCSKCGLGFVHRRKDTMSRGKKYHYRYYSCKTYKHTHELEKCGNKIWRADKLEELIINRVNNYSFASRNVDKEDELDSLNEKLKIEHAKKKRLFDLYINGSYEVSELDSMMNDIDAQINYYESQIEANEELKKNKKIQENLADLATVDFDSLEFREKQLYLKSLINKIYIDGEQVTIEWL

SEQ ID NO: 133 - protein, 485 aa, synthetic construct
  REGION 1..485: note = Synthetic: Wildtype TP901
MTKKVAIYTRVSTTNQAEEGFSIDEQIDRLTKYAEAMGWQVSDTYTDAGFSGAKLERPAMQRLINDIENKAFDTVLVYKLDRLSRSVRDTLYLVKDVFTKNKIDFISLNESIDTSSAMGSLFLTILSAINEFERENIKERMTMGKLGRAKSGKSMMWTKTAFGYYHNRKTGILEIVPLQATIVEQIFTDYLSGISLTKLRDKLNESGHIGKDIPWSYRTLRQTLDNPVYCGYIKFKDSLFEGMHKPIIPYETYLKVQKELEERQQQTYERNNNPRPFQAKYMLSGMARCGYCGAPLKIVLGHKRKDGSRTMKYHCANRFPRKTKGITVYNDNKKCDSGTYDLSNLENTVIDNLIGFQENNDSLLKIINGNNQPILDTSSFKKQISQIDKKIQKNSDLYLNDFITMDELKDRTDSLQAEKKLLKAKISENKFNDSTDVFELVKTQLGSIPINELSYDNKKKIVNNLVSKVDVTADNVDIIFKFQLA

SEQ ID NO: 134 - DNA, 38 bp, synthetic construct
  misc_feature 1..38: note = Synthetic: Consensus attB sequence
  source 1..38: mol_type = other DNA
ggtttgtnnacnacngnnnnnncngtngtnaggatcnn

SEQ ID NO: 135 - DNA, 38 bp, synthetic construct
  misc_feature 1..38: note = Synthetic: Consensus attB sequence
  source 1..38: mol_type = other DNA
nngatcctgacgacngnnnnnncngtngtnnacaaacc

SEQ ID NO: 136 - DNA, 38 bp, synthetic construct
  misc_feature 1..38: note = Synthetic: Consensus attB sequence
  source 1..38: mol_type = other DNA
nngatcctgacgacngnnnnnncngtngtnaggatcnn

SEQ ID NO: 137 - DNA, 48 bp, synthetic construct
  misc_feature 1..48: note = Synthetic: s5-1
  source 1..48: mol_type = other DNA
ccattccttcttctaccacagcgttctcaattgggtaagcatcaaaat

SEQ ID NO: 138 - DNA, 48 bp, synthetic construct
  misc_feature 1..48: note = Synthetic: s5-2
  source 1..48: mol_type = other DNA
tttagttgctcctgacagcagtgggagctattgtctaagagatacaaa

SEQ ID NO: 139 - DNA, 48 bp, synthetic construct
  misc_feature 1..48: note = Synthetic: s5-4
  source 1..48: mol_type = other DNA
ggagttgagtcctgactgcagcacatgcaggtgaggaagcagtagaag

SEQ ID NO: 140 - DNA, 48 bp, synthetic construct
  misc_feature 1..48: note = Synthetic: s5-6
  source 1..48: mol_type = other DNA
tactggggattcccataaccgtgcactcagctgcgggaaggcaggaga

SEQ ID NO: 141 - DNA, 48 bp, synthetic construct
  misc_feature 1..48: note = Synthetic: s5-8
  source 1..48: mol_type = other DNA
gagtagtgtatctgacaccagcacactctggtgtcagaatcttgggtt

SEQ ID NO: 142 - DNA, 48 bp, synthetic construct
  misc_feature 1..48: note = Synthetic: s5-10
  source 1..48: mol_type = other DNA
atagtgaaacttacaacacagagtttccagttgttcaatctctcttgt

SEQ ID NO: 143 - DNA, 48 bp, synthetic construct
  misc_feature 1..48: note = Synthetic: s5-11
  source 1..48: mol_type = other DNA
ttgaagccccttctcctacagagcaagcagcagggtaaattctgtatt

SEQ ID NO: 144 - DNA, 48 bp, synthetic construct
  misc_feature 1..48: note = Synthetic: s5-14
  source 1..48: mol_type = other DNA
tggtgcaatcttggctcactgcaacctctgcagttgaagcaattctcc

SEQ ID NO: 145 - DNA, 48 bp, synthetic construct
  misc_feature 1..48: note = Synthetic: s5-15
  source 1..48: mol_type = other DNA
atttgtggtactatactacagagaatgctgatgttaaagagctaattt

SEQ ID NO: 146 - DNA, 48 bp, synthetic construct
  misc_feature 1..48: note = Synthetic: s5-16
  source 1..48: mol_type = other DNA
gcactccagcctgggcaacagagcactctgttgtcaaaaaaaaaaaaa

SEQ ID NO: 147 - DNA, 48 bp, synthetic construct
  misc_feature 1..48: note = Synthetic: s5-17
  source 1..48: mol_type = other DNA
acacctggatctccacccctgtgtttgctgttgtctaagttttacatt

SEQ ID NO: 148 - DNA, 48 bp, synthetic construct
  misc_feature 1..48: note = Synthetic: s5-18
  source 1..48: mol_type = other DNA
ccgtcatgttcctcaccacagctaacccagtcttcaaaactcacttaa

SEQ ID NO: 149 - DNA, 48 bp, synthetic construct
  misc_feature 1..48: note = Synthetic: s5-19
  source 1..48: mol_type = other DNA
ggcctcctgcttgctcagctgagacggcagaggctgaagctgcacggg

SEQ ID NO: 150 - DNA, 48 bp, synthetic construct
  misc_feature 1..48: note = Synthetic: s5-21
  source 1..48: mol_type = other DNA
cttccttcttttaaaccactgatcatgcagaagtataagctcaccagt

SEQ ID NO: 151 - DNA, 48 bp, synthetic construct
  misc_feature 1..48: note = Synthetic: s5-24
  source 1..48: mol_type = other DNA
gtggcagaaatagtacaacagctaacgcagatgttaaaaatcaaatgg

SEQ ID NO: 152 - DNA, 48 bp, synthetic construct
  misc_feature 1..48: note = Synthetic: s1-1
  source 1..48: mol_type = other DNA
cagtgtgcttgttgcctacagcctctgcagaagttcacaaacttgcac

SEQ ID NO: 153 - DNA, 48 bp, synthetic construct
  misc_feature 1..48: note = Synthetic: s1-5
  source 1..48: mol_type = other DNA
attaggggttggtggcaatggagatctcagtggtgaaaaatcacttct

SEQ ID NO: 154 - DNA, 48 bp, synthetic construct
  misc_feature 1..48: note = Synthetic: s1-10
  source 1..48: mol_type = other DNA
atgtggttttgttcactgtagaatcctcagtggttagcacaatgttaa

SEQ ID NO: 155 - DNA, 48 bp, synthetic construct
  misc_feature 1..48: note = Synthetic: s1-11
  source 1..48: mol_type = other DNA
catttctcttgatgactgcagagtattccattgttgacaaatccattt

SEQ ID NO: 156 - DNA, 48 bp, synthetic construct
  misc_feature 1..48: note = Synthetic: s1-17
  source 1..48: mol_type = other DNA
gctatgggtttttaaacactgacttgcctggtgtgaggatccatgctt

SEQ ID NO: 157 - DNA, 48 bp, synthetic construct
  misc_feature 1..48: note = Synthetic: s1-25
  source 1..48: mol_type = other DNA
gtgttctgatacttgcaactgaacacactgtagtccacacaccactat

SEQ ID NO: 158 - DNA, 48 bp, synthetic construct
  misc_feature 1..48: note = Synthetic: s1-28
  source 1..48: mol_type = other DNA
gggggttgcttctcaccgtagatgaccctcttgtccacaaaccacaac

SEQ ID NO: 159 - DNA, 48 bp, synthetic construct
  misc_feature 1..48: note = Synthetic: s1-31
  source 1..48: mol_type = other DNA
cagcctggctcccaacctccgtcactgccgttgcaaacaaaccaaacc

SEQ ID NO: 160 - DNA, 48 bp, synthetic construct
  misc_feature 1..48: note = Synthetic: s1-39
  source 1..48: mol_type = other DNA
tttcaaagatcctgacctccgaagaagctggagttgagatcatactgt

SEQ ID NO: 161 - DNA, 48 bp, synthetic construct
  misc_feature 1..48: note = Synthetic: s1-41
  source 1..48: mol_type = other DNA
cgagcttgctcctcacagcagaggactctgaagacaggagcatcatga

SEQ ID NO: 162 - DNA, 48 bp, synthetic construct
  misc_feature 1..48: note = Synthetic: s1-43
  source 1..48: mol_type = other DNA
ccttgcagatctatacggctgactacacagtggtgagaaccatagtct

SEQ ID NO: 163 - DNA, 48 bp, synthetic construct
  misc_feature 1..48: note = Synthetic: s3-28
  source 1..48: mol_type = other DNA
acctttgtttggccccaactgcctctgccactgtcgacacacttggga

SEQ ID NO: 164 - DNA, 48 bp, synthetic construct
  misc_feature 1..48: note = Synthetic: s3-41
  source 1..48: mol_type = other DNA
gatgcatgattctaggcactgggaacacagtggtgatcaaaccagtca

SEQ ID NO: 165 - DNA, 84 bp, synthetic construct
  misc_feature 1..84: note = Synthetic: s5-1
  source 1..84: mol_type = other DNA
ggtttgtctggtcaaccaccgcgttctcagtggtgtacggtacaaaccttcgtttcaactgaccaattcactccaaatgtaaca

SEQ ID NO: 166 - DNA, 80 bp, synthetic construct
  misc_feature 1..80: note = Synthetic: s5-2
  source 1..80: mol_type = other DNA
ggtttgtctggtcaaccaccgcgggctcagtggtgtacggtacaaaccaagaggaaaagtatacctgcagcaagtgatat

SEQ ID NO: 167 - DNA, 88 bp, synthetic construct
  misc_feature 1..88: note = Synthetic: s5-4
  source 1..88: mol_type = other DNA
ggtttgtctggtcaaccaccgcgcactcagtggtgtacggtacaaaccattgctgccgtctgtccccttcccatcccttctgacatgt

SEQ ID NO: 168 - DNA, 77 bp, synthetic construct
  misc_feature 1..77: note = Synthetic: s5-6
  source 1..77: mol_type = other DNA
ggtttgtctggtcaaccaccgcgcactcagtggtgtacggtacaaaccagcccgaacttagcttccctcatcgccta

SEQ ID NO: 169 - DNA, 79 bp, synthetic construct
  misc_feature 1..79: note = Synthetic: s5-8
  source 1..79: mol_type = other DNA
ggtttgtctggtcaaccaccgcgcactcagtggtgtacggtacaaaccatatcgtttgctactcaaactgtgtggtcct

SEQ ID NO: 170 - DNA, 78 bp, synthetic construct
  misc_feature 1..78: note = Synthetic: s5-10
  source 1..78: mol_type = other DNA
ggtttgtctggtcaaccaccgcgttctcagtggtgtacggtacaaacctatcttccttttctgtggaagggaaaactt

SEQ ID NO: 171 - DNA, 85 bp, synthetic construct
  misc_feature 1..85: note = Synthetic: s5-11
  source 1..85: mol_type = other DNA
ggtttgtctggtcaaccaccgcgcactcagtggtgtacggtacaaaccaagcagctcggagcctctctgtcacttgctctttaga

SEQ ID NO: 172 - DNA, 97 bp, synthetic construct
  misc_feature 1..97: note = Synthetic: s5-14
  source 1..97: mol_type = other DNA
ggtttgtctggtcaaccaccgcgacctcagtggtgtacggtacaaaccgcctgtcacctcctgagtacctgggattacaggcgcacaacactactct

SEQ ID NO: 173 - DNA, 92 bp, synthetic construct
  misc_feature 1..92: note = Synthetic: s5-15
  source 1..92: mol_type = other DNA
ggtttgtctggtcaaccaccgcgaactcagtggtgtacggtacaaacctcaggaggtgtttttccttccttttgaaatcagatagcaggaaa

SEQ ID NO: 174 - DNA, 89 bp, synthetic construct
  misc_feature 1..89: note = Synthetic: s5-16
  source 1..89: mol_type = other DNA
ggtttgtctggtcaaccaccgcgcactcagtggtgtacggtacaaaccaagagcacagtgattatgattattgtttctgagtgatggca

SEQ ID NO: 175 - DNA, 93 bp, synthetic construct
  misc_feature 1..93: note = Synthetic: s5-17
  source 1..93: mol_type = other DNA
ggtttgtctggtcaaccaccgcgttctcagtggtgtacggtacaaacctaatgtccgcacttcctaagttttggtttccagagccactagtaa

SEQ ID NO: 176 - DNA, 99 bp, synthetic construct
  misc_feature 1..99: note = Synthetic: s5-18
  source 1..99: mol_type = other DNA
ggtttgtctggtcaaccaccgcgaactcagtggtgtacggtacaaacctctattcgcactcagggaggccttccctgatctccagggccagattaagta

SEQ ID NO: 177 - DNA, 107 bp, synthetic construct
  misc_feature 1..107: note = Synthetic: s5-19
  source 1..107: mol_type = other DNA
ggtttgtctggtcaaccaccgcgacctcagtggtgtacggtacaaacccggccagtcagtcactgtcaggtgagcacccgcgagggtcccggggaggtacgggtgac

SEQ ID NO: 178 - DNA, 91 bp, synthetic construct
  misc_feature 1..91: note = Synthetic: s5-21
  source 1..91: mol_type = other DNA
ggtttgtctggtcaaccaccgcgcactcagtggtgtacggtacaaaccaccttcacgtcctaaccagaccatgagctccaggggtaaaatc

SEQ ID NO: 179 - DNA, 84 bp, synthetic construct
  misc_feature 1..84: note = Synthetic: s5-24
  source 1..84: mol_type = other DNA
ggtttgtctggtcaaccaccgcgaactcagtggtgtacggtacaaacccatgtaaaaccagcttattgccaacagagttatgga

SEQ ID NO: 180 - DNA, 91 bp, synthetic construct
  misc_feature 1..91: note = Synthetic: s1-1
  source 1..91: mol_type = other DNA
ggtttgtctggtcaaccaccgcgtcctcagtggtgtacggtacaaaccagaatcaggataaggctcctctaggtacatgtggctaagctgc

SEQ ID NO: 181 - DNA, 92 bp, synthetic construct
  misc_feature 1..92: note = Synthetic: s1-5
  source 1..92: mol_type = other DNA
ggtttgtctggtcaaccaccgcgatctcagtggtgtacggtacaaaccggtcctccatcaagcgctgaggcacagtgaatgtactcagaggg

SEQ ID NO: 182 - DNA, 86 bp, synthetic construct
  misc_feature 1..86: note = Synthetic: s1-10
  source 1..86: mol_type = other DNA
ggtttgtctggtcaaccaccgcgtcctcagtggtgtacggtacaaaccacacaatagcgacatggagcaggtatcagtttacgttt

SEQ ID NO: 183 - DNA, 146 bp, synthetic construct
  misc_feature 1..146: note = Synthetic: s1-11
  source 1..146: mol_type = other DNA
ggtttgtctggtcaaccaccgcgtactcagtggtgtacggtacaaaccaaccacctgattgtgatggatgtttaggttgtttccaatcttttgctattaaaaagaatgtgacagtgagaaactttgtgtatagtgtcattttgcac

SEQ ID NO: 184 - DNA, 90 bp, synthetic construct
  misc_feature 1..90: note = Synthetic: s1-17
  source 1..90: mol_type = other DNA
ggtttgtctggtcaaccaccgcgttctcagtggtgtacggtacaaaccagcatctttcaaatagcacctcattttatcctgaagacccag

SEQ ID NO: 185 - DNA, 89 bp, synthetic construct
  misc_feature 1..89: note = Synthetic: s1-25
  source 1..89: mol_type = other DNA
ggtttgtctggtcaaccaccgcgcactcagtggtgtacggtacaaacccaaaactggtagaatctcagtgaccttaaagattgcctggg

SEQ ID NO: 186 - DNA, 110 bp, synthetic construct
  misc_feature 1..110: note = Synthetic: s1-28
  source 1..110: mol_type = other DNA
ggtttgtctggtcaaccaccgcggactcagtggtgtacggtacaaacccatctccctttttccgtgctgattaggactggggcagtggggttcgtgtatttgtgacctgg

SEQ ID NO: 187 - DNA, 87 bp, synthetic construct
  misc_feature 1..87: note = Synthetic: s1-31
  source 1..87: mol_type = other DNA
ggtttgtctggtcaaccaccgcgacctcagtggtgtacggtacaaaccgcagtgtccacctggcggaacctttctctccactatcca

SEQ ID NO: 188 - DNA, 83 bp, synthetic construct
  misc_feature 1..83: note = Synthetic: s1-39
  source 1..83: mol_type = other DNA
ggtttgtctggtcaaccaccgcggactcagtggtgtacggtacaaacctagtaaataggatcaaaggaatagagtctgatgcc

SEQ ID NO: 189 - DNA, 84 bp, synthetic construct
  misc_feature 1..84: note = Synthetic: s1-41
  source 1..84: mol_type = other DNA
ggtttgtctggtcaaccaccgcggactcagtggtgtacggtacaaaccctgtgactacatttagtgagcaggtggaatgaacaa

SEQ ID NO: 190 - DNA, 90 bp, synthetic construct
  misc_feature 1..90: note = Synthetic: s1-43
  source 1..90: mol_type = other DNA
ggtttgtctggtcaaccaccgcgtactcagtggtgtacggtacaaaccggataagtttcagtaaagggactactgcaggatttggtggta

SEQ ID NO: 191 - DNA, 91 bp, synthetic construct
  misc_feature 1..91: note = Synthetic: s3-28
  source 1..91: mol_type = other DNA
ggtttgtctggtcaaccaccgcgtcctcagtggtgtacggtacaaacctgcgttgtctgagctcctcagcactccctccccaacttcctat

SEQ ID NO: 192 - DNA, 93 bp, synthetic construct
  misc_feature 1..93: note = Synthetic: s3-41
  source 1..93: mol_type = other DNA
ggtttgtctggtcaaccaccgcgaactcagtggtgtacggtacaaaccagtcaccaatctggaggggatgaggtgagggagggatagacaata

SEQ ID NO: 193 - DNA, 47 bp, synthetic construct
  misc_feature 1..47: note = Synthetic: s5-1
  source 1..47: mol_type = other DNA
acacgacgctcttccgatctnnnnttctttcatttcgtgtgagggtc

SEQ ID NO: 194 - DNA, 46 bp, synthetic construct
  misc_feature 1..46: note = Synthetic: s5-2
  source 1..46: mol_type = other DNA
acacgacgctcttccgatctnnnnaccctgtgttagaagcagtaac

SEQ ID NO: 195 - DNA, 47 bp, synthetic construct
  misc_feature 1..47: note = Synthetic: s5-4
  source 1..47: mol_type = other DNA
acacgacgctcttccgatctnnnnatgtgtttggaagctgagcatag

SEQ ID NO: 196 - DNA, 46 bp, synthetic construct
  misc_feature 1..46: note = Synthetic: s5-6
  source 1..46: mol_type = other DNA
acacgacgctcttccgatctnnnncgatgtgttcctccatgaagca

SEQ ID NO: 197 - DNA, 49 bp, synthetic construct
  misc_feature 1..49: note = Synthetic: s5-8
  source 1..49: mol_type = other DNA
acacgacgctcttccgatctnnnngcatagctgagtaatacattgcatg

SEQ ID NO: 198 - DNA, 48 bp, synthetic construct
  misc_feature 1..48: note = Synthetic: s5-10
  source 1..48: mol_type = other DNA
acacgacgctcttccgatctnnnnatgggcagcattcattaagtaaat

SEQ ID NO: 199 - DNA, 48 bp, synthetic construct
  misc_feature 1..48: note = Synthetic: s5-11
  source 1..48: mol_type = other DNA
acacgacgctcttccgatctnnnncaattagttggctgtataatttgg

SEQ ID NO: 200 - DNA, 50 bp, synthetic construct
  misc_feature 1..50: note = Synthetic: s5-14
  source 1..50: mol_type = other DNA
acacgacgctcttccgatctnnnnatttcacattgattgattttcttgtt

SEQ ID NO: 201 - DNA, 47 bp, synthetic construct
  misc_feature 1..47: note = Synthetic: s5-15
  source 1..47: mol_type = other DNA
acacgacgctcttccgatctnnnnactgccacctacattcaacaaat

SEQ ID NO: 202 - DNA, 44 bp, synthetic construct
  misc_feature 1..44: note = Synthetic: s5-16
  source 1..44: mol_type = other DNA
acacgacgctcttccgatctnnnnagagggctgaggttgcaatg

SEQ ID NO: 203 - DNA, 52 bp, synthetic construct
  misc_feature 1..52: note = Synthetic: s5-17
  source 1..52: mol_type = other DNA
acacgacgctcttccgatctnnnntgcagataattcttatgcaaaacagaac

SEQ ID NO: 204 - DNA, 48 bp, synthetic construct
  misc_feature 1..48: note = Synthetic: s5-18
  source 1..48: mol_type = other DNA
acacgacgctcttccgatctnnnngcagaaagttagtatgacacagac

SEQ ID NO: 205 - DNA, 50 bp, synthetic construct
  misc_feature 1..50: note = Synthetic: s5-19
  source 1..50: mol_type = other DNA
acacgacgctcttccgatctnnnngaccagacagttccagatacattcca

SEQ ID NO: 206 - DNA, 48 bp, synthetic construct
  misc_feature 1..48: note = Synthetic: s5-21
  source 1..48: mol_type = other DNA
acacgacgctcttccgatctnnnnttactctctctcctagactcaagc

SEQ ID NO: 207 - DNA, 48 bp, synthetic construct
  misc_feature 1..48: note = Synthetic: s5-24
  source 1..48: mol_type = other DNA
acacgacgctcttccgatctnnnntgctagtatttgtctgcactttct

SEQ ID NO: 208 - DNA, 46 bp, synthetic construct
  misc_feature 1..46: note = Synthetic: s1-1
  source 1..46: mol_type = other DNA
acacgacgctcttccgatctnnnncgtgggccagttttaaatgtca

SEQ ID NO: 209 - DNA, 46 bp, synthetic construct
  misc_feature 1..46: note = Synthetic: s1-5
  source 1..46: mol_type = other DNA
acacgacgctcttccgatctnnnngctgaacattagctccatatat

SEQ ID NO: 210 - DNA, 49 bp, synthetic construct
  misc_feature 1..49: note = Synthetic: s1-10
  source 1..49: mol_type = other DNA
acacgacgctcttccgatctnnnnagtctctcatttaccagttttgctt

SEQ ID NO: 211 - DNA, 47 bp, synthetic construct
  misc_feature 1..47: note = Synthetic: s1-11
  source 1..47: mol_type = other DNA
acacgacgctcttccgatctnnnnggattatcccatatccagacata

SEQ ID NO: 212 - DNA, 46 bp, synthetic construct
  misc_feature 1..46: note = Synthetic: s1-17
  source 1..46: mol_type = other DNA
acacgacgctcttccgatctnnnnagcccagagttaaccaagctac

SEQ ID NO: 213 - DNA, 47 bp, synthetic construct
  misc_feature 1..47: note = Synthetic: s1-25
  source 1..47: mol_type = other DNA
acacgacgctcttccgatctnnnntacaactaaagcaccaatggctc

SEQ ID NO: 214 - DNA, 45 bp, synthetic construct
  misc_feature 1..45: note = Synthetic: s1-28
  source 1..45: mol_type = other DNA
acacgacgctcttccgatctnnnngtcagaagtcccagatgtgct

SEQ ID NO: 215 - DNA, 42 bp, synthetic construct
  misc_feature 1..42: note = Synthetic: s1-31
  source 1..42: mol_type = other DNA
acacgacgctcttccgatctnnnnggaatccgcctcctgacg

SEQ ID NO: 216 - DNA, 47 bp, synthetic construct
  misc_feature 1..47: note = Synthetic: s1-39
  source 1..47: mol_type = other DNA
acacgacgctcttccgatctnnnntgatgttagagatcagcctgtac

SEQ ID NO: 217 - DNA, 47 bp, synthetic construct
  misc_feature 1..47: note = Synthetic: s1-41
  source 1..47: mol_type = other DNA
acacgacgctcttccgatctnnnnagccatttccttcctagcaaatt

SEQ ID NO: 218 - DNA, 48 bp, synthetic construct
  misc_feature 1..48: note = Synthetic: s1-43
  source 1..48: mol_type = other DNA
acacgacgctcttccgatctnnnncaatacaggcacaatccccttatt

SEQ ID NO: 219 - DNA, 46 bp, synthetic construct
  misc_feature 1..46: note = Synthetic: s3-28
  source 1..46: mol_type = other DNA
acacgacgctcttccgatctnnnntgaagggaaaggtctggcttta

SEQ ID NO: 220 - DNA, 43 bp, synthetic construct
  misc_feature 1..43: note = Synthetic: s3-41
  source 1..43: mol_type = other DNA
acacgacgctcttccgatctnnnncagcagtcgatgtgggaac

SEQ ID NO: 221 - DNA, 45 bp, synthetic construct
  misc_feature 1..45: note = Synthetic: s5-1
  source 1..45: mol_type = other DNA
gacgtgtgctcttccgatcttgttacatttggagtgaattggtca

SEQ ID NO: 222 - DNA, 46 bp, synthetic construct
  misc_feature 1..46: note = Synthetic: s5-2
  source 1..46: mol_type = other DNA
gacgtgtgctcttccgatctatatcacttgctgcaggtatactttt

SEQ ID NO: 223 - DNA, 42 bp, synthetic construct
  misc_feature 1..42: note = Synthetic: s5-4
  source 1..42: mol_type = other DNA
gacgtgtgctcttccgatctacatgtcagaagggatgggaag

SEQ ID NO: 224 - DNA, 42 bp, synthetic construct
  misc_feature 1..42: note = Synthetic: s5-6
  source 1..42: mol_type = other DNA
gacgtgtgctcttccgatcttaggcgatgagggaagctaagt

SEQ ID NO: 225 - DNA, 42 bp, synthetic construct
  misc_feature 1..42: note = Synthetic: s5-8
  source 1..42: mol_type = other DNA
gacgtgtgctcttccgatctaggaccacacagtttgagtagc

SEQ ID NO: 226 - DNA, 41 bp, synthetic construct
  misc_feature 1..41: note = Synthetic: s5-10
  source 1..41: mol_type = other DNA
gacgtgtgctcttccgatctaagttttcccttccacagaaa

SEQ ID NO: 227 - DNA, 44 bp, synthetic construct
  misc_feature 1..44: note = Synthetic: s5-11
  source 1..44: mol_type = other DNA
gacgtgtgctcttccgatcttctaaagagcaagtgacagagagg

SEQ ID NO: 228 - DNA, 40 bp, synthetic construct
  misc_feature 1..40: note = Synthetic: s5-14
  source 1..40: mol_type = other DNA
gacgtgtgctcttccgatctagagtagtgttgtgcgcctg

SEQ ID NO: 229 - DNA, 46 bp, synthetic construct
  misc_feature 1..46: note = Synthetic: s5-15
  source 1..46: mol_type = other DNA
gacgtgtgctcttccgatcttttcctgctatctgatttcaaaagga

SEQ ID NO: 230 - DNA, 45 bp, synthetic construct
  misc_feature 1..45: note = Synthetic: s5-16
  source 1..45: mol_type = other DNA
gacgtgtgctcttccgatcttgccatcactcagaaacaataatca

SEQ ID NO: 231 - DNA, 42 bp, synthetic construct
  misc_feature 1..42: note = Synthetic: s5-17
  source 1..42: mol_type = other DNA
gacgtgtgctcttccgatctttactagtggctctggaaacca

SEQ ID NO: 232 - DNA, 43 bp, synthetic construct
  misc_feature 1..43: note = Synthetic: s5-18
  source 1..43: mol_type = other DNA
gacgtgtgctcttccgatcttacttaatctggccctggagatc

SEQ ID NO: 233 - DNA, 38 bp, synthetic construct
  misc_feature 1..38: note = Synthetic: s5-19
  source 1..38: mol_type = other DNA
gacgtgtgctcttccgatctgtcacccgtacctccccg

SEQ ID NO: 234 - DNA, 42 bp, synthetic construct
  misc_feature 1..42: note = Synthetic: s5-21
  source 1..42: mol_type = other DNA
gacgtgtgctcttccgatctgattttacccctggagctcatg

SEQ ID NO: 235 - DNA, 43 bp, synthetic construct
  misc_feature 1..43: note = Synthetic: s5-24
  source 1..43: mol_type = other DNA
gacgtgtgctcttccgatcttccataactctgttggcaataag

SEQ ID NO: 236 - DNA, 42 bp, synthetic construct
  misc_feature 1..42: note = Synthetic: s1-1
  source 1..42: mol_type = other DNA
gacgtgtgctcttccgatctgcagcttagccacatgtaccta

SEQ ID NO: 237 - DNA, 40 bp, synthetic construct
  misc_feature 1..40: note = Synthetic: s1-5
  source 1..40: mol_type = other DNA
gacgtgtgctcttccgatctccctctgagtacattcactg

SEQ ID NO: 238 - DNA, 43 bp, synthetic construct
  misc_feature 1..43: note = Synthetic: s1-10
  source 1..43: mol_type = other DNA
gacgtgtgctcttccgatctaaacgtaaactgatacctgctcc

SEQ ID NO: 239 - DNA, 45 bp, synthetic construct
  misc_feature 1..45: note = Synthetic: s1-11
  source 1..45: mol_type = other DNA
gacgtgtgctcttccgatctgtgcaaaatgacactatacacaaag

SEQ ID NO: 240 - DNA, 44 bp, synthetic construct
  misc_feature 1..44: note = Synthetic: s1-17
  source 1..44: mol_type = other DNA
gacgtgtgctcttccgatctctgggtcttcaggataaaatgagg

SEQ ID NO: 241 - DNA, 42 bp, synthetic construct
  misc_feature 1..42: note = Synthetic: s1-25
  source 1..42: mol_type = other DNA
gacgtgtgctcttccgatctcccaggcaatctttaaggtcac

SEQ ID NO: 242 - DNA, 41 bp, synthetic construct
  misc_feature 1..41: note = Synthetic: s1-28
  source 1..41: mol_type = other DNA
gacgtgtgctcttccgatctccaggtcacaaatacacgaac

SEQ ID NO: 243 - DNA, 43 bp, synthetic construct
  misc_feature 1..43: note = Synthetic: s1-31
  source 1..43: mol_type = other DNA
gacgtgtgctcttccgatcttggatagtggagagaaaggttcc

SEQ ID NO: 244 - DNA, 40 bp, synthetic construct
  misc_feature 1..40: note = Synthetic: s1-39
  source 1..40: mol_type = other DNA
gacgtgtgctcttccgatctggcatcagactctattcctt

SEQ ID NO: 245 - DNA, 42 bp, synthetic construct
  misc_feature 1..42: note = Synthetic: s1-41
  source 1..42: mol_type = other DNA
gacgtgtgctcttccgatctttgttcattccacctgctcact

SEQ ID NO: 246 - DNA, 42 bp, synthetic construct
  misc_feature 1..42: note = Synthetic: s1-43
  source 1..42: mol_type = other DNA
gacgtgtgctcttccgatcttaccaccaaatcctgcagtagt

SEQ ID NO: 247 - DNA, 42 bp, synthetic construct
  misc_feature 1..42: note = Synthetic: s3-28
  source 1..42: mol_type = other DNA
gacgtgtgctcttccgatctataggaagttggggagggagtg

SEQ ID NO: 248 - DNA, 43 bp, synthetic construct
  misc_feature 1..43: note = Synthetic: s3-41
  source 1..43: mol_type = other DNA
gacgtgtgctcttccgatcttattgtctatccctccctcacct

SEQ ID NO: 249 - protein, 22 aa, synthetic construct
  REGION 1..22: note = Synthetic: Bxb1 recombinase construct C terminal end amino acid sequence
GSGSGSHHHHHHGSGPKKKRKV

SEQ ID NO: 250 - DNA, 44 bp, synthetic construct
  misc_feature 1..44: note = Synthetic: P1
  source 1..44: mol_type = other DNA
acacgacgctcttccgatctnnnnatgcttctctgcatccgtct

SEQ ID NO: 251 - DNA, 45 bp, synthetic construct
  misc_feature 1..45: note = Synthetic: P2
  source 1..45: mol_type = other DNA
gacgtgtgctcttccgatctagggaatagatgacatcaaagagaa

SEQ ID NO: 252 - DNA, 38 bp, synthetic construct
  misc_feature 1..38: note = Synthetic: attB
  source 1..38: mol_type = other DNA
ggtttgtcgccgacggcggtctccgtcggcaacaaacc

SEQ ID NO: 253 - DNA, 38 bp, synthetic construct
  misc_feature 1..38: note = Synthetic: attB
  source 1..38: mol_type = other DNA
ggtttgtcggcgacggcggtctccgtcgccaacaaacc

SEQ ID NO: 254 - DNA, 38 bp, synthetic construct
  misc_feature 1..38: note = Synthetic: attB
  source 1..38: mol_type = other DNA
ggtttgtcgtcgacggcggtctccgtcgacaacaaacc

SEQ ID NO: 255 - DNA, 48 bp, synthetic construct
  misc_feature 1..48: note = Synthetic: attP
  source 1..48: mol_type = other DNA
ggtttgtctggtcacccaccgcggtctcagtggggtacggtacaaacc

SEQ ID NO: 256 - DNA, 48 bp, synthetic construct
  misc_feature 1..48: note = Synthetic: attP
  source 1..48: mol_type = other DNA
ggtttgtctggtcagccaccgcggtctcagtggcgtacggtacaaacc

SEQ ID NO: 257 - DNA, 48 bp, synthetic construct
  misc_feature 1..48: note = Synthetic: attP
  source 1..48: mol_type = other DNA
ggtttgtctggtcatccaccgcggtctcagtggagtacggtacaaacc

SEQ ID NO: 258 - DNA, 38 bp, synthetic construct
  misc_feature 1..38: note = Synthetic: attB
  source 1..38: mol_type = other DNA
ggtttgtcgaagacggcggtctccgtcttcaacaaacc

SEQ ID NO: 259 - DNA, 38 bp, synthetic construct
  misc_feature 1..38: note = Synthetic: attB
  source 1..38: mol_type = other DNA
ggtttgtcgaggacggcggtctccgtcctcaacaaacc

SEQ ID NO: 260 - DNA, 38 bp, synthetic construct
  misc_feature 1..38: note = Synthetic: attB
  source 1..38: mol_type = other DNA
ggtttgtcgatgacggcggtctccgtcatcaacaaacc

SEQ ID NO: 261 - DNA, 48 bp, synthetic construct
  misc_feature 1..48: note = Synthetic: attP
  source 1..48: mol_type = other DNA
ggtttgtctggtcaaacaccgcggtctcagtgttgtacggtacaaacc

SEQ ID NO: 262 - DNA, 48 bp, synthetic construct
  misc_feature 1..48: note = Synthetic: attP
  source 1..48: mol_type = other DNA
ggtttgtctggtcaagcaccgcggtctcagtgctgtacggtacaaacc

SEQ ID NO: 263 - DNA, 48 bp, synthetic construct
  misc_feature 1..48: note = Synthetic: attP
  source 1..48: mol_type = other DNA
ggtttgtctggtcaatcaccgcggtctcagtgatgtacggtacaaacc

SEQ ID NO: 264 - DNA, 48 bp, synthetic construct
  misc_feature 1..48: note = Synthetic: s5-8
  source 1..48: mol_type = other DNA
aacccaagattctgacaccagagtgtgctggtgtcagatacactactc

SEQ ID NO: 265 - DNA, 48 bp, synthetic construct
  misc_feature 1..48: note = Synthetic: s5-1
  source 1..48: mol_type = other DNA
attttgatgcttacccaattgagaacgctgtggtagaagaaggaatgg

SEQ ID NO: 266 - DNA, 48 bp, synthetic construct
  misc_feature 1..48: note = Synthetic: s5-11
  source 1..48: mol_type = other DNA
aatacagaatttaccctgctgcttgctctgtaggagaaggggcttcaa

SEQ ID NO: 267 - DNA, 48 bp, synthetic construct
  misc_feature 1..48: note = Synthetic: s1-41
  source 1..48: mol_type = other DNA
tcatgatgctcctgtcttcagagtcctctgctgtgaggagcaagctcg

SEQ ID NO: 268 - DNA, 38 bp, synthetic construct; identical to SEQ ID NO: 80
  misc_feature 1..38: note = Synthetic: Symmetric AttB site
  source 1..38: mol_type = other DNA

SEQ ID NO: 269 - DNA, 38 bp, synthetic construct
  misc_feature 1..38: note = Synthetic: Symmetric AttB site
  source 1..38: mol_type = other DNA
ggtttgttgacgacggagaccgccgtcgtcgacaaacc

SEQ ID NO: 270 - DNA, 48 bp, synthetic construct
  misc_feature 1..48: note = Synthetic: s5-16
  source 1..48: mol_type = other DNA
tttttttttttttgacaacagagtgctctgttgcccaggctggagtgc

SEQ ID NO: 271 - DNA, 48 bp, synthetic construct
  misc_feature 1..48: note = Synthetic: s3-28
  source 1..48: mol_type = other DNA
tcccaagtgtgtcgacagtggcagaggcagttggggccaaacaaaggt